(19) United States
(12) Patent Application Publication
(10) Pub. No.: US 2024/0308931 A1
(43) Pub. Date: Sep. 19, 2024

(54) LIGAND-CONTROLLED DIVERGENT DEHYDROGENATIVE REACTIONS OF ALIPHATIC ACIDS

(71) Applicant: THE SCRIPPS RESEARCH INSTITUTE, La Jolla, CA (US)

(72) Inventors: Jin-Quan YU, San Diego, CA (US); Zhen WANG, San Diego, CA (US); Liang HU, San Diego, CA (US)

(21) Appl. No.: 18/578,559

(22) PCT Filed: Jul. 14, 2022
(86) PCT No.: PCT/US22/37089

§ 371 Date: Jan. 11, 2024

Related U.S. Application Data
(60) Provisional application No. 63/203,241, filed on Jul. 14, 2021.

(51) Int. Cl. C07B 35/04; B01J 23/44; C07B 37/10; C07C 51/377; C07C 67/317

(52) U.S. Cl. CPC C07B 35/04; B01J 23/44; C07B 37/10; C07C 51/377; C07C 67/317

(57) ABSTRACT

Disclosed herein are palladium-catalyzed dehydrogenation processes of carboxylic acids to make α, β-unsaturated carboxylic acids or γ-alkylidene butenolides. The processes allow the chemoselective dehydrogenation of carboxylic acids in the presence of other enolizable functionalities such as ketones, providing reactivity that is inaccessible with existing carbonyl desaturation protocols.

### CROSS-REFERENCE TO RELATED APPLICATIONS

This application claims the benefit of priority to U.S. Provisional Patent Application No. 63/203,241, filed on Jul. 14, 2021, which application is incorporated in its entirety as if fully set forth herein.

### STATEMENT OF GOVERNMENT SUPPORT

This invention was made with government support under grant number R01GM084019 awarded by the National Institutes of Health. The government has certain rights in the invention.

### BACKGROUND

Dehydrogenation of aliphatic chains is an important process in both the bulk chemical industry and fine chemical synthesis. The development of synthetically useful dehydrogenation reactions via C—H activation without installing exogeneous directing groups faces two challenges: the difficulty of activating methylene C—H bonds, and product inhibition by, or overreaction of the olefin products. Earlier work focused on ligand-enabled C—H activation reactions directed by native functional groups such as free carboxylic acids, free aliphatic amines, and native amides (1). However, development of methylene C—H activation reactions of acyclic aliphatic substrates directed by innate functionalities such as carboxylic acids remain an unsolved problem (2, 3).

While the development of dehydrogenation reactions via methylene C—H activation has been hampered by the aforementioned challenges, alternative approaches have been developed to meet the synthetic needs for desaturation of carbonyl compounds (4). Exploration of syn-eliminations of selenoxide and sulfoxide intermediates has led to useful dehydrogenation tools in synthesis (5-7). Other organic reagents such as N-tert-butyl phenylsulfinimidoyl chloride, hypervalent iodine and N-oxoammonium salts have also been developed to prepare enones (8-10). Electrochemically driven desaturation of carbonyl compounds was also recently achieved (11). An extensively researched catalytic pathway for dehydrogenation is the formation of Pd(II) enolates from ketones, with subsequent β-hydride elimination affording enone products (12-15). Also known is a method to desaturate free carboxylic acids via preformation of zinc enediolates which are subsequently oxidized to α,β-unsaturated acids by Pd(II) complexes with allyl acetate serving as the stoichiometric oxidant (16). Yet, as reported, zinc enediolates of α-branched carboxylic acids gave limited conversion.

In 2006, Goldman and Brookhart reported an example of alkane dehydrogenation via methylene C—H activation (17): the challenge of product inhibition was addressed by coupling the dehydrogenation with a tandem metathesis and hydrogenation reaction. Although this tandem catalytic system provides a promising strategy for converting low-MW alkanes to high-MW alkanes, it has proven so far difficult to extend this concept to the development of dehydrogenative transformations using synthetically relevant substrates as limiting reagents. Recently, dehydrogenation of cyclooctane by a combination of cobalt and tungsten catalysts via a radical pathway has also been observed, albeit in modest yields (18). An effort to develop Pd(II)-catalyzed dehydrogenation reactions using substrates as limiting reagent via C—H activation entailed the use of oxazoline directing groups with aliphatic acids. However, the synthetic utility of these transformations is limited by extra steps required to install and remove a directing group (19), and moreover, the reaction is also limited to a single cyclopentanecarboxylic acid substrate.

### SUMMARY

The present disclosure addresses these problems and others by providing processes of Pd-catalyzed dehydrogenation reactions that convert free aliphatic carboxylic acids into either α,β-unsaturated acids or γ-alkylidene butenolides, respectively. Thus, in an embodiment, the present disclosure provides a process for making a compound of formula (2):

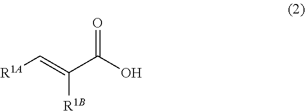

The process comprises contacting a compound of formula (1):

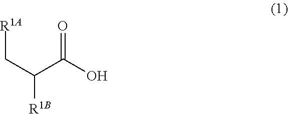

with a source of Pd(II), optionally in the presence of an oxidant, and a ligand of formula (L-1):

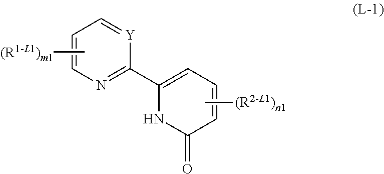

whereby the compound of formula (2) is formed.

In this embodiment, Y is N or CH.

In some embodiments, substituents R^{1A} and R^{1B} are independently selected from the group consisting of H, C₁-C₆-alkyl, C₃-C₂₀-cycloalkyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), 5- to 10-membered heteroaryl and —(C₁-C₆-alkyl)-(5- to 10-membered heteroaryl) (wherein 1-4 heteroaryl members are independently selected from N, O, and S).

In other embodiments, R^{1A} and R^{1B}, together with the carbon atoms to which they are bound, form a 5- to 6-membered carbocyclic ring.

R^{1A} and R^{1B}, or the carbocyclic ring that they form, are independently and optionally substituted with one to five substituents selected from the group consisting of halo, C₁-C₆-alkyl, C₁-C₆-haloalkyl, C₁-C₆-alkoxy, —S—(C₁-C₆-alkyl), C₆-C₁₀-aryloxy, —O—(C₁-C₆-alkyl)(C₆-C₁₀-aryl), —S(O)₀₋₂(C₆-C₁₀-aryl) (optionally substituted with C₁-C₆-alkyl), C(O)NR^{A}R^{B}, NR^{A}C(O)O(C₁-C₆-alkyl), —C(O)(C₁-C₆-alkyl), —C(O)O(C₁-C₆-alkyl), —C(O)(C₆-C₁₀-aryl), and —C(O)O(C₆-C₁₀-aryl).

Subscript m1 is selected from 0, 1, 2, 3, and 4, and subscript n1 is selected from 0, 1, 2, and 3.

R^{1-L1} and R^{2-L1} are independently selected from the group consisting of —CN, halo, NR^{A}R^{B}, C₁-C₆-alkyl, C₁-C₆-haloalkyl, C₂-C₆-alkenyl, C₂-C₆-alkynyl, C₁-C₆-alkoxy, C₁-C₆-haloalkoxy, C(O)C₁-C₆-alkyl, C(O)NR^{A}R^{B}, S(O)NR^{A}R^{B}, S(O)₂NR^{A}R^{B}, C₃-C₁₄-cycloalkyl, C₆-C₁₀-aryl, C₆-C₁₀-aryloxy, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S).

Each alkyl, aryl, cycloalkyl, heterocycloalkyl, and heteroaryl moiety of R^{1-L1} and R^{2-L1} is optionally substituted with one to four substituents selected from the group consisting of halo, oxo, C₁-C₆-alkyl, C₁-C₆-haloalkyl, C₁-C₆-alkoxy, C(O)NR^{A}R^{B}, C₁-C₆-alkoxy, C₆-C₁₀-aryl (optionally substituted by one to three halo and C₁-C₆-alkyl), and 5- to 10-membered heteroaryl (wherein 1-4 heteroaryl members are independently selected from N, O, and S; optionally substituted by one to three substituents selected from C₁-C₆-alkyl and 5- to 10-membered heteroaryl).

R^{A} and R^{B} are independently selected from the group consisting of H, C₁-C₆-alkyl, C₁-C₆-haloalkyl, —C₁-C₆-alkyl-C₆-C₁₀-aryl, C(O)C₁-C₆-alkyl, C(O)C₁-C₆-alkyl-C₆-C₁₀-aryl, C(O)OC₁-C₆-alkyl, C₆-C₁₀-aryl (optionally fused to C₃-C₁₄-cycloalkyl that is optionally substituted by one to four halo and C₁-C₆-alkyl), and wherein each aryl is optionally substituted with one to three substituents selected from C₁-C₆-alkyl, halo, C₁-C₆-haloalkyl, and 3- to 14-membered heterocycloalkyl (wherein 1-4 ring members are independently selected from N, O, and S); each alkyl is optionally substituted with one to three substituents selected from halo, NRR′ (wherein R and R′ are independently selected from H, C₁-C₆-alkyl, C(O)C₁-C₆-alkyl, and C(O)C₆-C₁₀-aryl).

In some embodiments wherein m1 is 2, then two adjacent R^{1-L1} together with the ring carbon atoms to which they are bound form a fused phenyl ring that is optionally substituted with one to three substituents selected the group consisting of halo, C₁-C₆-alkyl, C₁-C₆-haloalkyl, C₁-C₆-alkoxy, and C₁-C₆-haloalkoxy.

The present disclosure also provides in another embodiment a process for making a compound of formula (5) or (7):

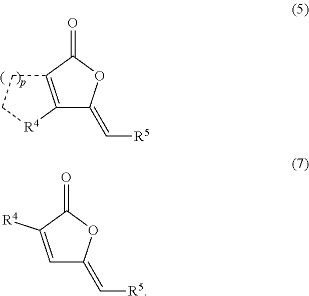

The process comprises contacting a compound of formula (3) or (6), respectively:

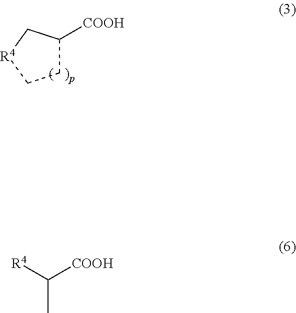

with a source of Pd(II), a ligand of formula (L-2):

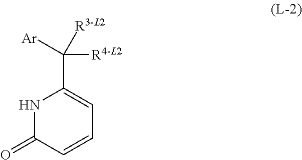

and a compound of formula (A):

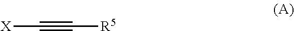

whereby the compound of formula (5) or (7) is formed.

In Formula (3), the dotted lines “ [symbol] ” represent optional single bonds. When the bonds are present then p is 1, 2, 3, or 4; and R⁴ is —CH₂—, wherein the resulting 5- to 8-membered ring is cycloalkyl or heterocycloalkyl (wherein 1-2 ring members are independently selected from N, O, and S). The ring is optionally substituted with one to three substituents selected from halo, C₁-C₆-alkyl, C₁-C₆-haloalkyl, C₁-C₆-alkoxy, —S(O)₀₋₂(C₆-C₁₀-aryl) (optionally substituted with C₁-C₆-alkyl), and C₆-C₁₀-aryl (optionally and independently substituted by one to three halo, C₁-C₆-alkyl, and C₁-C₆-haloalkyl).

When the bonds “ [symbol] ” are not present, then R⁴ is selected from the group consisting of C₁-C₂₀-alkyl, C₃-C₁₄-cycloalkyl, —(C₁-C₆-alkyl)-(C₃-C₁₄-cycloalkyl), C₆-C₁₀-aryl, —(C₁-C₆-alkyl)-(C₆-C₁₀-aryl), 3- to 14-membered heterocycloalkyl and —(C₁-C₆-alkyl)-(3- to 14-membered heterocycloalkyl) (wherein 1-4 ring members are independently selected from N, O, and S), 5- to 10-membered heteroaryl and —(C₁-C₆-alkyl)-(5- to 10-membered heteroaryl) (wherein 1-4 heteroaryl members are independently selected from N, O, and S).

Substituent R⁴ or the ring in which R⁴ is a member is independently and optionally substituted with one to five substituents selected from the group consisting of halo, C₁-C₆-alkyl, C₁-C₆-haloalkyl, C₁-C₆-alkoxy, —S—(C₁-C₆-alkyl), C₆-C₁₀-aryloxy, —O—(C₁-C₆-alkyl)(C₆-C₁₀-aryl), —S(O)₀₋₂(C₆-C₁₀-aryl) (optionally substituted with C₁-C₆-alkyl), C(O)NR^{A}R^{B}, NR^{A}C(O)O(C₁-C₆-alkyl), —C(O)(C₁-C₆-alkyl), —C(O)O(C₁-C₆-alkyl), —C(O)(C₆-C₁₀-aryl), and —C(O)O(C₆-C₁₀-aryl).

R⁵ is selected from the group consisting of —SiR₃ and —CH(OSiR₃)R′. Each R and R′ is independently selected from the group consisting of C₁-C₆-alkyl, C₁-C₆-alkoxy, C₁-C₆-haloalkyl, and C₃-C₁₄-cycloalkyl.

Ar is

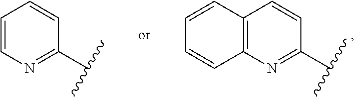 [SMILES: *c1ccccn1 ; *c1ccc2ccccc2n1]

and is optionally substituted with one to three substituents selected from the group consisting of halo, C₁-C₆-alkyl, and C₁-C₆-alkoxy.

In some embodiments, R^{3-L2} and R^{4-L2} are independently selected from the group consisting of H, OH, halo, C₁-C₆-alkyl, C₁-C₆-haloalkyl, C₃-C₁₄-cycloalkyl, —(C₁-C₆-alkyl)-(C₃-C₁₄-cycloalkyl), C₆-C₁₀-aryl, and —(C₁-C₆-alkyl)-(C₆-C₁₀-aryl).

In other embodiments, R^{3-L2} and R^{4-L2}, together with the carbon atom to which they are bound, form a 5- or 6-membered cycloalkyl.

In still other embodiments, one of R^{3-L2} and R^{4-L2}, together with the 3-pyridyl carbon when Ar is pyridyl, form a 5- to 6-membered cycloalkyl fused to the pyridyl.

Substituents R^{3-L2} and R^{4-L2}, or a ring in which either is a member, are independently and optionally substituted by one to three substituents selected from the group consisting of halo, C₁-C₆-alkyl, C₁-C₆-haloalkyl, and C₁-C₆-alkoxy.

### DETAILED DESCRIPTION

The processes disclosed herein are ligand-enabled Pd(II)-catalyzed divergent dehydrogenation reactions useful for directly converting a wide range of aliphatic carboxylic acids into α,β-unsaturated acids or γ-alkylidene butenolides via activation of either methylene or methyl C—H bonds. The processes overcome the problem of product inhibition by use of ligands with different bite angles resulting in either the prevention of vinyl C—H activation of the typically more reactive α,β-unsaturated acids, or providing a tandem vinyl C—H activation/alkynyl bromide coupling leading to butenolides that mask the carboxylic acid moiety from further reactions. The directed nature of the processes allows chemoselective dehydrogenation of carboxylic acids in the presence of other enolizable functionalities such as ketones, providing reactivity that is inaccessible with existing carbonyl desaturation protocols.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C H(CH₂CH₃)₂, —CH₂CH₂C(CH₃)₃, —CH₂CH₂C(CH₂CH₃)₃, —CH(CH₃)CH₂CH(CH₃)₂, —CH(CH₃) CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples include a (C₂-C₈)alkynyl group, such as acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O— neopentyl, —O-hexyl, —O-isohexyl, and —O-neohexyl.

The term “cycloalkyl” refers to a saturated monocyclic, bicyclic, tricyclic, or polycyclic, 3- to 20-membered ring system, such as a C₃-C₂₀-cycloalkyl or C₃-C₈-cycloalkyl. The cycloalkyl may be attached via any atom. Representative examples of cycloalkyl include, but are not limited to cyclopropyl, cyclobutyl, cyclopentyl, and cyclohexyl. Polycyclic cycloalkyl includes rings that can be fused, bridged, and/or spiro-fused. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. Polycyclic heterocycloalkyl includes rings that can be fused, bridged, and/or spiro-fused. In addition, a heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

The term “oxo” refers to a ═O atom bound to an atom that is part of a saturated or unsaturated moiety. Thus, the ═O atom can be bound to a carbon, sulfur, or nitrogen atom that is part of a cyclic or acyclic moiety.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. An illustration of tautomerism includes the following:

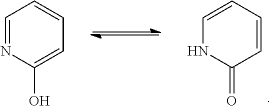 [SMILES: Oc1ccccn1 ; O=c1cccc[nH]1]

The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As summarized above, the present disclosure provides in an embodiment a process for making a compound of formula (2):

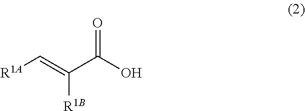

The process comprises contacting a compound of formula (1):

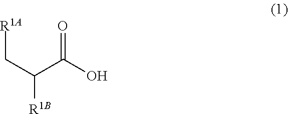

with a source of Pd(II), optionally in the presence of an oxidant, and a ligand of formula (L-1):

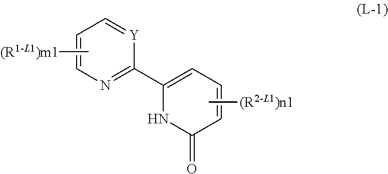

whereby the compound of formula (2) is formed.

In some embodiments, R^{B} is H and R^{1A} is other than H as defined herein. Examples of compounds of formula (1) are illustrated in the following table.

TABLE
Specific examples of compounds of formula (1).

| | |
|---|---|
| 1a | 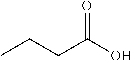 |
| 1b | 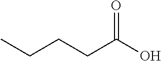 |
| 1c | 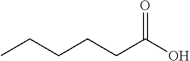 |
| 1d | 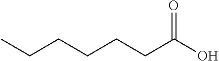 |
| 1e | 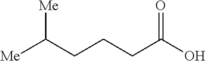 |
| 1f | 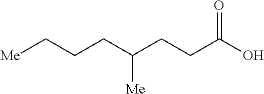 [SMILES: CCCCC(C)CCC(=O)O] |
| 1g | 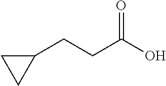 |
| 1h | 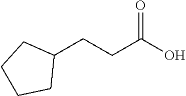 |
| 1i | 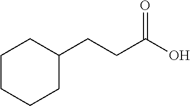 [SMILES: O=C(O)CCC1CCCCC1] |
| 1j | 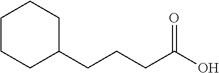 [SMILES: O=C(O)CCCC1CCCCC1] |
| 1k | 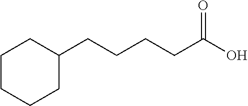 [SMILES: O=C(O)CCCCC1CCCCC1] |
| 1l | 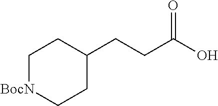 [SMILES: CC(C)(C)OC(=O)N1CCC(CCC(=O)O)CC1] |
| 1m | 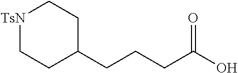 [SMILES: Cc1ccc(S(=O)(=O)N2CCC(CCCC(=O)O)CC2)cc1] |
| 1n | 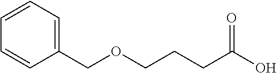 [SMILES: O=C(O)CCCOCc1ccccc1] |
| 1o | 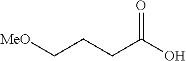 |
| 1p | 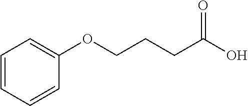 [SMILES: O=C(O)CCCOc1ccccc1] |
| 1q | 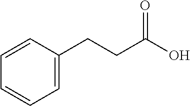 [SMILES: O=C(O)CCc1ccccc1] |
| 1r | 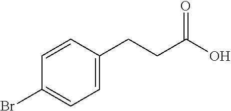 [SMILES: O=C(O)CCc1ccc(Br)cc1] |
| 1s | 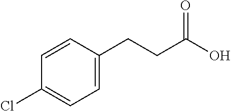 [SMILES: O=C(O)CCc1ccc(Cl)cc1] |
| 1t | 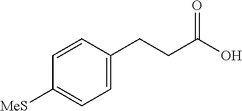 [SMILES: CSc1ccc(CCC(=O)O)cc1] |
| 1u | 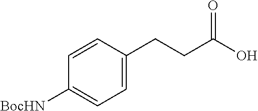 [SMILES: CC(C)(C)OC(=O)Nc1ccc(CCC(=O)O)cc1] |
| 1v | 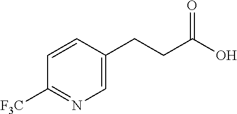 [SMILES: O=C(O)CCc1ccc(C(F)(F)F)nc1] |
| 1w | 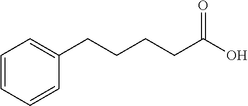 [SMILES: O=C(O)CCCCc1ccccc1] |
| 1x | 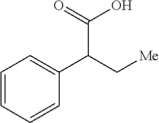 [SMILES: CCC(C(=O)O)c1ccccc1] |
| 1y | 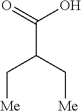 |
| 1z | 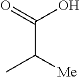 |
| 1aa | 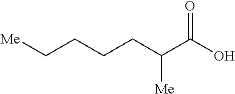 |
| 1ab | 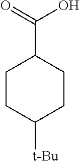 [SMILES: CC(C)(C)C1CCC(C(=O)O)CC1] |
| 1ac | 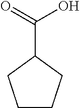 |
| 1ad | 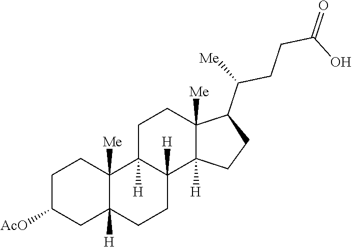 |
| 1ae | 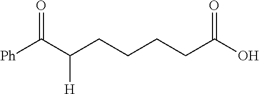 [SMILES: O=C(O)CCCCCC(=O)c1ccccc1] |
| 1af | 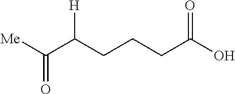 |
| 1ag | 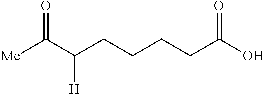 [SMILES: CC(=O)CCCCCC(=O)O] |
| 1ah | 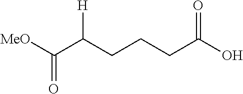 [SMILES: COC(=O)CCCCC(=O)O] |
| lai | 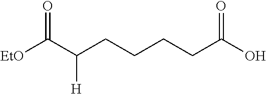 [SMILES: CCOC(=O)CCCCCC(=O)O] |

In various embodiments, Y in formula (L-1) is CH. In other embodiments, Y is N.

In additional embodiments, n1 is 0. Optionally in combination with any other embodiment described herein, another embodiment provides ligands of formula (L-1) wherein m1 is 0, 1, or 2.

In formula (L-1), in accordance with various embodiments, R^{1-L1} is selected from the group consisting of halo, C₁-C₆-alkyl, C₁-C₆-haloalkyl, and C₁-C₆-alkoxy.

In other embodiments, m1 is 2. Where two adjacent R^{1-L1} exist, they, together with the ring carbon atoms to which they are bound, form a fused phenyl ring that is optionally substituted as described herein.

The amount of ligand (L-1) can vary between about 1 and 25 mol % (based upon formula (1)). In various embodiments, the amount is between about 2 and 20 mol %, 3 and 18 mol %, 5 and 15 mol %, or 8 and 12 mol %. Illustrative amounts of ligand include about 5, 10, 11, 12, 13, 14, 15, 16, 17, 18, 20, and 25 mol %.

In illustrative embodiments, the present disclosure provides ligands of formula (L-1) selected from the table below.

TABLE
Specific examples of ligands of formula (L-1).

| | |
|---|---|
| L8 | 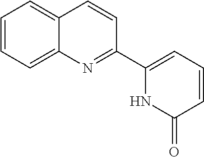 [SMILES: O=c1cccc(-c2ccc3ccccc3n2)[nH]1] |
| L9 | 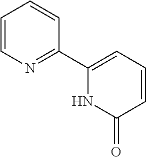 [SMILES: O=c1cccc(-c2ccccn2)[nH]1] |
| L10 | 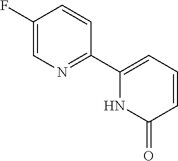 [SMILES: O=c1cccc(-c2ccc(F)cn2)[nH]1] |
| L11 | 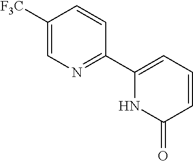 [SMILES: O=c1cccc(-c2ccc(C(F)(F)F)cn2)[nH]1] |
| L12 | 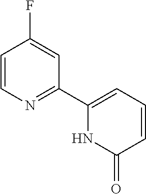 [SMILES: O=c1cccc(-c2cc(F)ccn2)[nH]1] |
| L13 | 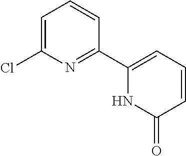 [SMILES: O=c1cccc(-c2cccc(Cl)n2)[nH]1] |
| L14 | 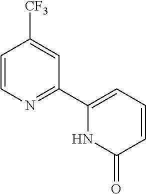 [SMILES: O=c1cccc(-c2cc(C(F)(F)F)ccn2)[nH]1] |
| L15 | 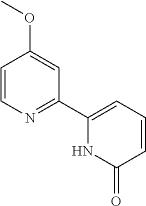 |
| L16 | 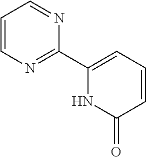 [SMILES: O=c1cccc(-c2ncccn2)[nH]1] |
| L17 | 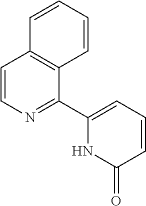 [SMILES: O=c1cccc(-c2nccc3ccccc23)[nH]1] |
| L18 | 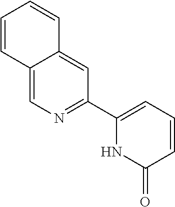 [SMILES: O=c1cccc(-c2cc3ccccc3cn2)[nH]1] |
| L19 | 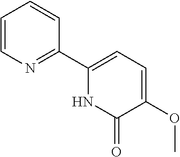 |

The present disclosure also provides in another embodiment, as summarized above, a process for making a compound of formula (5) or (7):

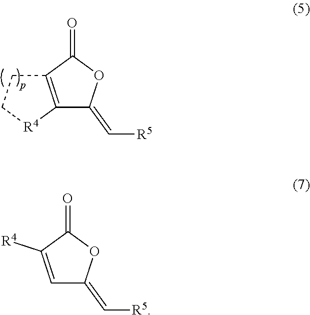

The process comprises contacting a compound of formula (3) or (6), respectively:

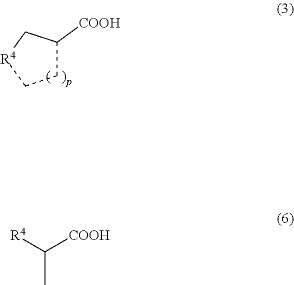

with a source of Pd(II), a ligand of formula (L-2):

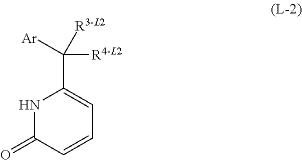

and a compound of formula (A):

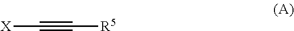

whereby the compound of formula (5) or (7) is formed.

In an embodiment, the contacting is between a compound of formula (3), source of Pd(II), ligand of formula (L-2), and compound of formula (A), whereby the compound of formula (5) is formed. In some embodiments, the optional bonds “ [symbol] ” are absent.

In other embodiments, the bonds “ [symbol] ” are present. In these embodiments, the fused [symbol] ring represented by the bonds and R⁴ can vary in size, as prescribed by subscript p. An exemplary ring is a 5- or 6-membered ring, wherein p is 1 or 2, respectively.

Illustrative compounds of formula (3), in yet additional embodiments, are shown in the table below.

TABLE
Specific examples of compounds of formula (3).

| | |
|---|---|
| 3a | 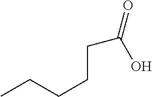 |
| 3b | 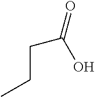 |
| 3c | 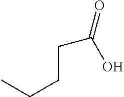 |
| 3d | 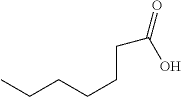 |
| 3e | 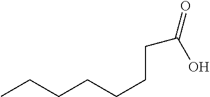 |
| 3f | 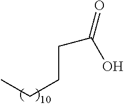 |
| 3g | 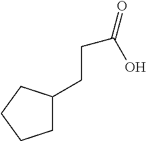 |
| 3h | 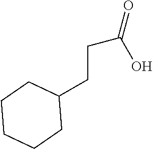 [SMILES: O=C(O)CCC1CCCCC1] |
| 3i | 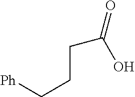 [SMILES: O=C(O)CCCc1ccccc1] |
| 3j | 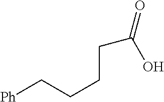 [SMILES: O=C(O)CCCCc1ccccc1] |
| 3k | 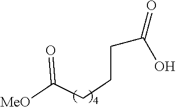 |
| 3l | 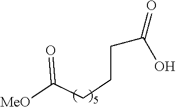 |
| 3m | 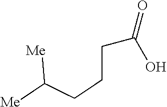 |
| 3n | 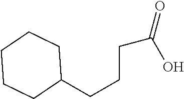 [SMILES: O=C(O)CCCC1CCCCC1] |
| 3o | 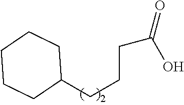 |
| 3p | 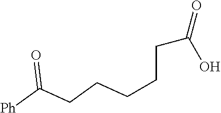 [SMILES: O=C(O)CCCCCC(=O)c1ccccc1] |
| 3q | 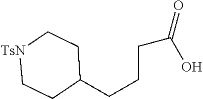 [SMILES: Cc1ccc(S(=O)(=O)N2CCC(CCCC(=O)O)CC2)cc1] |
| 3r | 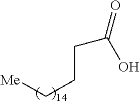 |
| 3s | 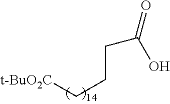 |
| 3t | 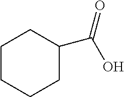 |
| 3u | 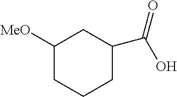 [SMILES: COC1CCCC(C(=O)O)C1] |
| 3v | 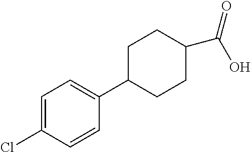 [SMILES: O=C(O)C1CCC(c2ccc(Cl)cc2)CC1] |
| 3w | 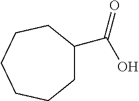 |
| 3x | 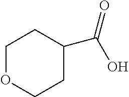 |
| 3y | 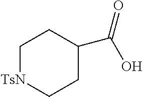 [SMILES: Cc1ccc(S(=O)(=O)N2CCC(C(=O)O)CC2)cc1] |
| 3z | 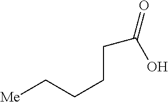 |

In another embodiment, the contacting is between a compound of formula (6), source of Pd(II), ligand of formula (L-2), and compound of formula (A). Examples of formula (6) compounds, per various embodiments, include those in the following table.

TABLE
Specific examples of compounds of formula (6).

| | |
|---|---|
| 6a | 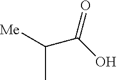 |
| 6b | 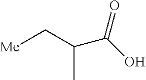 |
| 6c | 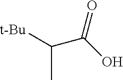 |
| 6d | 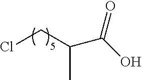 |
| 6e | 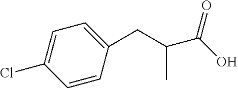 |
| 6f | 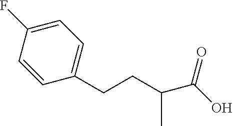 |
| 6g | 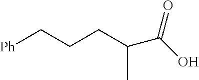 |
| 6h | 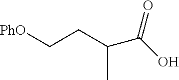 |
| 6i | 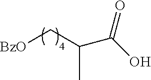 |
| 6j | 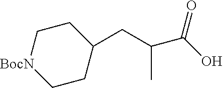 [SMILES: CC(CC1CCN(C(=O)OC(C)(C)C)CC1)C(=O)O] |

In the ligand of formula (L-2), concerning the process for making a compound of formula (5) or (7), according to an embodiment, Ar is

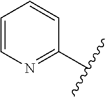 [SMILES: *c1ccccn1]

that is optionally substituted as described herein. In another embodiment, Ar is

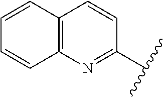 [SMILES: *c1ccc2ccccc2n1]

that is optionally substituted as described herein.

In still further embodiments, R^{3-L2} and R^{4-L2} are independently selected from optionally substituted C₁-C₆-alkyl and —(C₁-C₆-alkyl)-(C₆-C₁₀-aryl). Illustrating these and other structural features of the ligand of formula (L-2) are examples in the table below.

TABLE
Specific examples of ligands of formula (L-2).

| | |
|---|---|
| L20 | 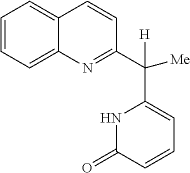 [SMILES: CC(c1ccc2ccccc2n1)c1cccc(=O)[nH]1] |
| L5 | 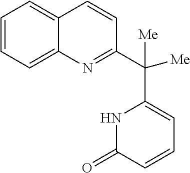 [SMILES: CC(C)(c1ccc2ccccc2n1)c1cccc(=O)[nH]1] |
| L21 | 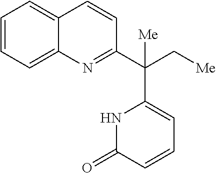 [SMILES: CCC(C)(c1ccc2ccccc2n1)c1cccc(=O)[nH]1] |
| L22 | 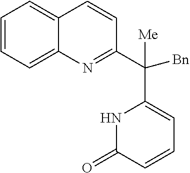 [SMILES: CC(Cc1ccccc1)(c1ccc2ccccc2n1)c1cccc(=O)[nH]1] |
| L23 | 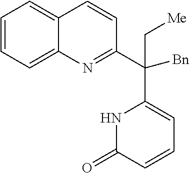 [SMILES: CCC(Cc1ccccc1)(c1ccc2ccccc2n1)c1cccc(=O)[nH]1] |
| L24 | 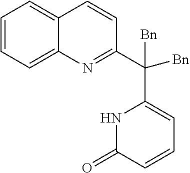 [SMILES: O=c1cccc(C(Cc2ccccc2)(Cc2ccccc2)c2ccc3ccccc3n2)[nH]1] |
| L25 | 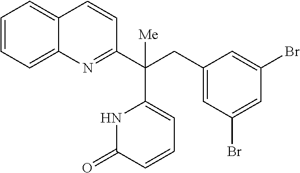 [SMILES: CC(Cc1cc(Br)cc(Br)c1)(c1ccc2ccccc2n1)c1cccc(=O)[nH]1] |
| L26 | 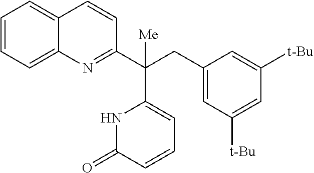 [SMILES: CC(C)(C)c1cc(CC(C)(c2ccc3ccccc3n2)c2cccc(=O)[nH]2)cc(C(C)(C)C)c1] |
| L27 | 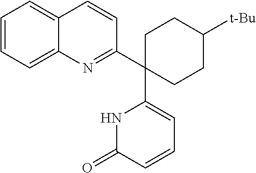 [SMILES: CC(C)(C)C1CCC(c2ccc3ccccc3n2)(c2cccc(=O)[nH]2)CC1] |
| L28 | 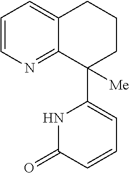 [SMILES: CC1(c2cccc(=O)[nH]2)CCCc2cccnc21] |
| L29 | 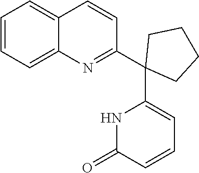 [SMILES: O=c1cccc(C2(c3ccc4ccccc4n3)CCCC2)[nH]1] |
| L30 | 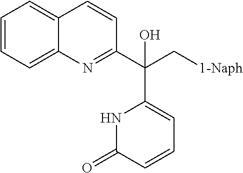 |
| L31 | 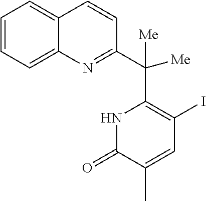 [SMILES: Cc1cc(I)c(C(C)(C)c2ccc3ccccc3n2)[nH]c1=O] |
| L32 | 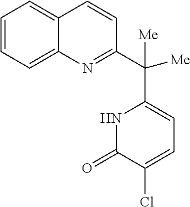 [SMILES: CC(C)(c1ccc2ccccc2n1)c1ccc(Cl)c(=O)[nH]1] |
| L33 | 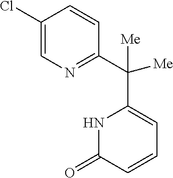 [SMILES: CC(C)(c1ccc(Cl)cn1)c1cccc(=O)[nH]1] |
| L34 | 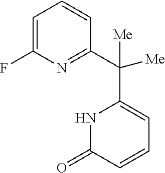 [SMILES: CC(C)(c1cccc(F)n1)c1cccc(=O)[nH]1] |
| L35 | 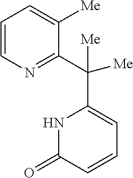 [SMILES: Cc1cccnc1C(C)(C)c1cccc(=O)[nH]1] |
| L36 | 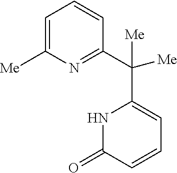 [SMILES: Cc1cccc(C(C)(C)c2cccc(=O)[nH]2)n1] |
| L37 | 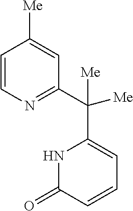 [SMILES: Cc1ccnc(C(C)(C)c2cccc(=O)[nH]2)c1] |
| L38 | 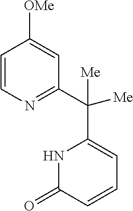 [SMILES: COc1ccnc(C(C)(C)c2cccc(=O)[nH]2)c1] |
| L39 | 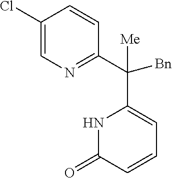 [SMILES: CC(Cc1ccccc1)(c1ccc(Cl)cn1)c1cccc(=O)[nH]1] |

The amount of ligand (L-2) can vary between about 1 and 25 mol % (based upon formula (3) or (6)). In various embodiments, the amount is between about 2 and 20 mol %, 3 and 18 mol %, 5 and 15 mol %, or 8 and 12 mol %. Illustrative amounts of ligand include about 5, 10, 11, 12, 13, 14, 15, 16, 17, 18, 20, and 25 mol %.

In various embodiments of the process for making a compound of formula (5) or (7), the halo-alkyne compound of formula (A) is one wherein R⁵ is —SiR₃. Various silyl groups —SiR₃ are known in the art and suitable for use in the process. Examples include those wherein each R is independently a straight or branched C₁-C₆-alkyl, such as methyl, ethyl, propyl and iso-propyl, and butyl and tert-butyl. A specific example is —Si(Pr)₃ (TIPS). In various embodiments, X in formula (A) is Br. An illustrative halo-alkyne of formula (A) is TIPS-CC—Br.

The cascade process for making compounds of formula (5) or formula (7) was possible using well-documented bromoalkynes as coupling partners to form complex 7-alkylidene butenolides (25). The development of synthetic methods for the construction of such scaffold has been ongoing for decades given the ubiquity of butenolides in natural products and bioactive molecules (26-29). Yet existing methods invariably require multiple synthetic steps consequential from conventional retrosynthetic approaches. The process described herein, based on an unconventional C—H dehydrogenation-alkynylation-cyclization cascade with aliphatic carboxylic acids, offers a novel retrosynthetic disconnection for the one-step construction of 7-alkylidene butenolides.

In the processes described herein, the contacting step utilizes a source of Pd(II), typically at catalytic loadings in various embodiments. Sources of palladium (II) can arise via reagents known in the art or commercially available. For example, one convenient source of palladium (II), per an embodiment, is Pd(OAc)₂. In additional embodiments, the source is Pd(CH₃CN)₄(BF₄)₂, PdCl₂, Pd(TFA)₂, Pd(CH₃CN)₂Cl₂, [Pd(allyl)Cl]₂, or Pd(PPh₃)₂Cl₂.

Palladium (II) loading can vary in accordance with factors known to those skilled in the art, such as overall reaction kinetics. Thus, in various embodiments, the source of palladium (II) is present in an amount of about 1 to about 15 mol % based upon the amount of compound of formula (2). In other embodiments, the amount is from about 7 to about 12 mol %. Exemplary amounts include 1, 2, 3, 4, 5, 6, 7, 8, 9, 10, 11, 12, 13, 14, and 15 mol %. In an embodiment, the amount is 10 mol %.

In various embodiments of the processes of the present disclosure, optionally in combination with any other embodiment described herein, the contacting step occurs in the presence of an oxidant or combination of oxidants described herein. In some embodiments, the oxidant is O₂. In other embodiments, the oxidant is an organic peroxide or organic hydroperoxide of general formula R′—O—O—R″, wherein R′ and R″ are chosen from H, C₁-C₆-alkyl, —(C₁-C₆-alkyl)-(C₆-C₁₀-aryl), —C(O)(C₁-C₆-alkyl), and —C(O)(C₆-C₁₀-aryl). Many useful organic peroxides are known in the organic chemistry art, and some are illustrated throughout the examples herein. They include tert-butylhydroperoxide (TBHP), cumene hydroperoxide (CMHP), acetyl-tert-butylperoxide (AcOOtBu), benzoyl tert-butylperoxide (BzOOtBu), dibenzoylperoxide (BzOOBz), and di-tertbutyl peroxide (tBuOOtBu). An exemplary oxidant is tert-butylhydroperoxide (TBHP) or O₂.

In some embodiments, the oxidant is at least one silver salt. Examples of suitable silver salts include but are not limited to Ag₂CO₃, AgOAc, silver pivalate (AgOPiv), and Ag₂O. An illustrative silver salt, per one embodiment, is Ag₂CO₃. Amounts of the silver salt An exemplary amount is 2 eq.

Amounts of the oxidant, according to various embodiments can vary, such as between 0.2 and 5 equivalents (eq), or between 1 and 4 eq., based upon the compound of formula (1), (3), or (6). In various embodiments, the oxidant is present in 0.5 equivalent (eq), 1 eq., 1.5 eq., 2 eq., 2.5 eq., or 3 eq. In embodiments wherein the oxidant is O₂, the processes can be carried out under 1.0, 1.5, 2.0, 2.5, or 3.0 atmospheres O₂.

In some embodiments, the oxidant is a combination of two, three, or four oxidants described herein. In this context, an additional oxidant, per some embodiments, is 1,4-benzoquinone (BQ). Various oxidant combinations are contemplated with any other embodiment herein. Illustrative oxidant combinations include TBHP/BQ and O₂/BQ.

In still further embodiments, the contacting step of the processes disclosed herein further occurs in the presence of at least one non-nucleophilic base. Some embodiments entail the use of two or three bases in combination. Exemplary non-nucleophilic bases are selected from the group consisting of KOAc, NaOAc, NaHCO₃, Na₂CO₃, NaH₂PO₄, Na₂HPO₄, Na₃PO₄, Li₂CO₃, LiOAc, Li₃PO₄, KOAc, KHCO₃, K₂CO₃, K₃PO₄, K₂IPO₄, KH₂PO₄, and CsOAc. In various embodiments, the contacting step occurs in the presence of Ag₂CO₃ and at least one non-nucleophilic base. These combinations include Ag₂CO₃ and NaOAc; Ag₂CO₃ and Li₂CO₃; and Ag₂CO₃, NaOAc, and Li₂CO₃. The amount of non-nucleophilic base can vary, such as between 1 and 5 equivalents (eq.) based upon the compound of formula (1), (3), or (6). Exemplary amounts in illustrative embodiments include 1, 2, 3, and 4 eq.

In some embodiments, the processes described herein are performed at elevated temperature. Useful temperatures include those between about 50 and 120 C, about 70 and 115 C, and about 80 and 110 C. Exemplary process temperatures include about 50, 60, 70, 80, 90, 100, and 110 C.

### EXAMPLES

Additional embodiments of the disclosure include the following non-limiting examples.

General Information. Pd(OAc)₂ was purchased from Strem. Solvents were obtained from Sigma-Aldrich, Alfa-Aesar, and Acros, and used directly without further purification. Other reagents were purchased at the highest commercial quality and used without further purification, unless otherwise stated. Analytical thin layer chromatography was performed on 0.25 mm silica gel 60-F254 or Merck pre-coated aluminium-backed silica gel F254 plates. ¹H NMR spectra were recorded on Bruker AMX-400, Bruker AV-500, or Bruker DRX-600 instruments. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 and were fully decoupled by broad band proton decoupling. Chemical shifts were referenced to the appropriate residual solvent peaks. Column chromatography was performed using E. Merck silica (60, particle size 0.043-0.063 mm), and pTLC was performed on Merck silica plates (60F-254). Reversed-phase chromatography was carried out automated using Biotage Isolera™ one with Biotage SNAP Samplet (C18) or SiliaSep Flash Cartridges (C18). High-resolution mass spectra (HRMS) were recorded on an Agilent Mass spectrometer using ESI-TOF (electrospray ionization-time of flight).

### Scheme for Preparing Bidentate Pyridine-Pyridone Ligands L8-L19 of Formula (L-1)

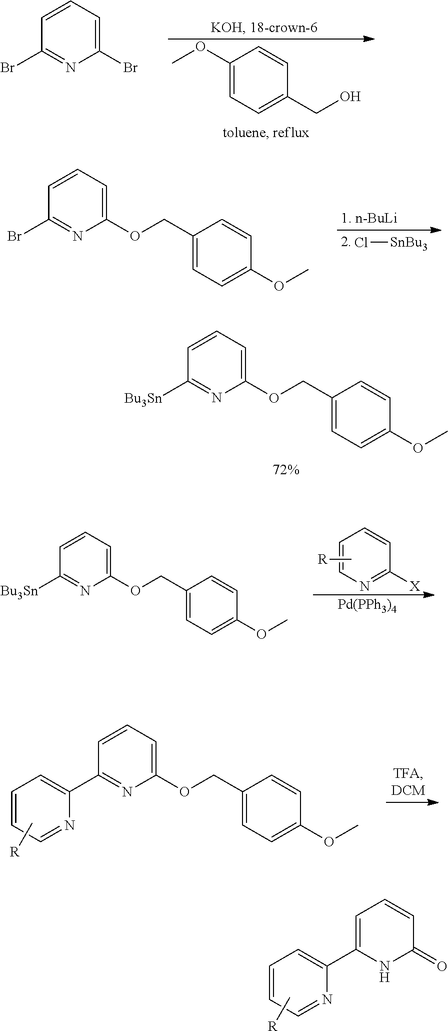

### Synthesis of Formula (L-1) Intermediate Compounds

Synthesis of 2-bromo-6-((4-methoxybenzyl)oxy)pyridine. A stirred mixture of 2,6-dibromopyridine (11.7 g, 50 mmol, 1.0 equiv), 4-methoxybenzyl alcohol (14.7 g, 50 mmol, 1.0 equiv), potassium hydroxide (3.37 g, 60 mmol, 1.2 equiv), 18-crown-6 (1.32 g, 5 mmol, 0.1 equiv) and toluene (200 mL) was heated under reflux for 12 hours. The cooled solution was evaporated under vacuum before 200 mL DCM was added. The organic layer was washed with brine twice and dried over anhydrous Na₂SO₄. A white solid was collected without further purification after evaporation of the solvent.

Synthesis of 2-((4-methoxybenzyl)oxy)-6-(tributylstannyl)pyridine. To a solution of 2-bromo-6-((4-methoxybenzyl)oxy)pyridine (14.6 g, 50 mmol, 1.0 equiv) in THE (200 mL) was added n-BuLi (22 mL, 55 mmol, 1.1 equiv) and the mixture was stirred at −78° C. for 30 min under nitrogen atmosphere. Then n-Bu₃SnCl (19.5 g, 60 mmol, 1.2 equiv) was added and the mixture was stirred at the same temperature for another 2 h. Saturated ammonium chloride solution (150 mL) was added to the solution and extracted with ethyl acetate (150 mL×3). The combined organic layers were dried over Na₂SO₄, filtered and concentrated in vacuo. The crude product was briefly purified by flash chromatography (hexane:ethyl acetate=10:1) to afford 2-((4-methoxybenzyl)oxy)-6-(tributylstannyl)pyridine (18.2 g, 72% over 2 steps) as a colorless oil.

### 2-((4-methoxybenzyl)oxy)-6-(tributylstannyl)pyridine

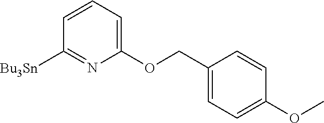

Colorless oil, ¹H NMR (500 MHz, Chloroform-d) δ 7.43-7.40 (m, 3H), 7.01 (dd, J=6.8, 1.0 Hz, 1H), 6.92-6.89 (m, 2H), 6.61 (dd, J=8.4, 1.0 Hz, 1H), 5.37 (s, 2H), 3.82 (s, 3H), 1.64-1.57 (m, 6H), 1.40-1.34 (m, 6H), 1.14-1.08 (m, 6H), 0.91 (t, J=7.4 Hz, 9H).

### Synthesis of Ligands L8-L19

General procedure: a mixture of 2-((4-methoxybenzyl)oxy)-6-(tributylstannyl)pyridine (10 mmol), corresponding 2-bromo-pyridine or 2-chloro-pyridine (10 mmol), and tetrakis(triphenylphosphine)-palladium(0) (1.15 g, 1.0 mmol) in 20 mL of toluene was refluxed under nitrogen for 48 h. The resulting brown mixture was evaporated in vacuo, and the dark mixture was purified over flash chromatography (hexane/EA) to afford the corresponding bipyridine compound. The corresponding bipyridine was dissolved in DCM and 1 mL of trifluoroacetic acid was added. The mixture was stirred under room temperature for 1 hour before 10 mL of saturated NaHCO₃ was added. The mixture was extracted with CHCl₃ (30 mL×3) and the organic layers were combined and concentrated under vacuum. The corresponding pyridine-pyridone ligand was purified by flash chromatography (EA:methanol=10:1).

### Example 1: Optimized Procedure for the Preparation of 6-(quinolin-2-yl)pyridin-2(1H)-one L8

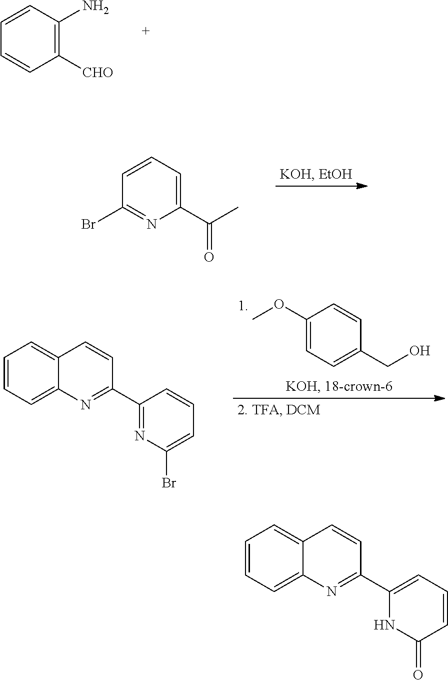

To a stirred solution of freshly bought 2-aminobenzaldehyde (2.4 g, 20 mmol) in EtOH (150 mL) at 25° C. was added 1-(6-bromopyridin-2-yl)ethan-1-one (1.0 equiv, 4.0 g, 20 mmol) and KOH (0.6 eq, 0.7 g, 12 mmol). The reaction mixture was stirred at 85° C. for 12 h. After cooling to room temperature, the mixture was evaporated under reduced pressure. Depending on the quality of 2-aminobenzaldehyde, the product 2-(6-bromopyridin-2-yl)quinoline can be used for the next step without further purification if >90% conversion is observed. Flash chromatography (hexane:ethyl acetate=10:1 to 4:1) gives a white solid if purification is needed.

A stirred mixture of 2-(6-bromopyridin-2-yl)quinoline (5.6 g, 20 mmol), 4-methoxybenzyl alcohol (2.8 g, 20 mmol, 1.0 equiv), potassium hydroxide (1.3 g, 24 mmol, 1.2 equiv), 18-crown-6 (0.5 g, 2 mmol, 0.1 equiv) and toluene (100 mL) was heated under reflux for 12 hours. The cooled solution was evaporated under vacuum before 200 mL DCM was added. The organic layer was washed with brine twice and dried over anhydrous Na₂SO₄. The combined DCM solution was then transferred into a flask and 2 mL of trifluoroacetic acid was added. The mixture was stirred under room temperature for 1 hour before 60 mL of saturated NaHCO₃ was added. The mixture was extracted with CHCl₃ (60 mL×3) and the organic layers were combined and concentrated under vacuum. L8 is purified by flash chromatography (EA:methanol=20:1) to afford a pale-yellow compound (71% over 3 steps).

This reaction procedure is adapted and optimized from a reported synthesis. (30)

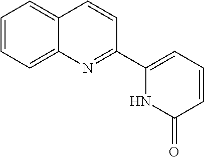 [SMILES: O=c1cccc(-c2ccc3ccccc3n2)[nH]1]

Yellow solid, ¹H NMR (600 MHz, Methanol-d₄) δ 8.41 (d, J=8.7 Hz, 1H), 8.14 (d, J=8.7 Hz, 1H), 8.09 (d, J=8.7 Hz, 1H), 7.94 (d, J=8.2 Hz, 1H), 7.81 (t, J=7.6 Hz, 1H), 7.74 (t, J=8.0 Hz, 1H), 7.71-7.60 (m, 1H), 7.30 (d, J=7.5 Hz, 1H), 6.67 (d, J=9.2 Hz, 1H). ¹³C NMR (151 MHz, MeOD) δ 163.92, 147.45, 147.00, 142.18, 137.89, 130.40, 129.05, 128.43, 127.66, 127.54, 120.84, 117.10, 105.96. HRMS (ESI-TOF) Calcd for C₁₀H₉N₂O [M+H]⁺: 221.0715; found: 221.0717.

### Example 2: [2,2′-bipyridin]-6(1H-one (L9)

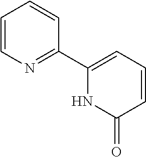 [SMILES: O=c1cccc(-c2ccccn2)[nH]1]

Yellow solid, ¹H NMR (600 MHz, Methanol-d4) δ 8.72 (dt, J=4.9, 1.3 Hz, 1H), 8.08 (dd, J=7.9, 1.3 Hz, 1H), 7.96 (td, J=7.8, 1.8 Hz, 1H), 7.73 (dd, J=9.1, 7.0 Hz, 1H), 7.49 (ddd, J=7.6, 4.9, 1.1 Hz, 1H), 7.18 (d, J=7.0 Hz, 1H), 6.65 (d, J=9.1 Hz, 1H). ¹³C NMR (151 MHz, MeOD) δ 163.53, 148.71, 147.59, 142.18, 141.76, 137.22, 124.35, 119.92, 119.52, 104.25. HRMS (ESI-TOF) Calcd for C₁₀H₉N₂O [M+H]⁺: 173.0715; found: 173.0717.

### Example 3: 5′-fluoro-[2,2′-bipyridin]-6(1H)-one (L10)

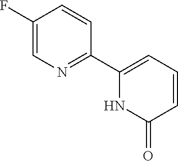 [SMILES: O=c1cccc(-c2ccc(F)cn2)[nH]1]

Yellow solid, ¹H NMR (600 MHz, Methanol-d₄) δ 8.63 (d, J=2.9 Hz, 1H), 8.15 (dd, J=8.9, 4.2 Hz, 1H), 7.81-7.75 (m, 1H), 7.74-7.70 (m, 1H), 7.15 (d, J=7.1 Hz, 1H), 6.63 (d, J=9.0 Hz, 1H). ¹³C NMR (151 MHz, Methanol-d₄) δ 163.58, 159.77 (d, J=258.6 Hz), 144.30, 141.69, 137.05, 136.88, 123.96 (d, J=19.3 Hz), 121.64 (d, J=5.3 Hz), 119.33, 104.24. HRMS (ESI-TOF) Calcd for C₁₀H₈FN₂O [M+H]⁺: 191.0621; found: 191.0623.

### Example 4: 5′-(trifluoromethyl)-[2,2′-bipyridin]-6(1H)-one (L11)

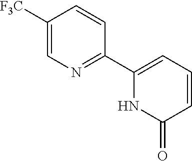 [SMILES: O=c1cccc(-c2ccc(C(F)(F)F)cn2)[nH]1]

Yellow solid, ¹H NMR (600 MHz, Methanol-d₄) δ 9.03 (m, 1H), 8.26 (m, 2H), 7.74 (t, J=8.1 Hz, 1H), 7.29 (d, J=7.1 Hz, 1H), 6.70 (d, J=9.1 Hz, 1H). ¹³C NMR (151 MHz, Methanol-d₄) δ 163.43, 151.49, 145.54 (q, J=4.2 Hz), 141.32, 138.52, 134.46 (q, J=3.4 Hz), 126.27 (q, J=33.3 Hz), 122.97 (q, J=271.5 Hz), 120.80, 119.99, 105.90. HRMS (ESI-TOF) Calcd for C₁₁H₈F₃N₂O [M+H]⁺: 241.0589; found: 241.0589.

### Example 5: 4′-fluoro-[2,2′-bipyridin]-6(1H)-one (L12)

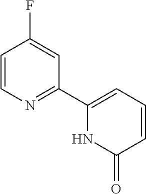 [SMILES: O=c1cccc(-c2cc(F)ccn2)[nH]1]

Yellow solid, ¹H NMR (600 MHz, Methanol-d₄) δ 8.72 (dd, J=8.3, 5.6 Hz, 1H), 7.93 (dd, J=10.0, 2.4 Hz, 1H), 7.72 (dd, J=9.1, 7.0 Hz, 1H), 7.31 (ddd, J=8.2, 5.6, 2.4 Hz, 1H), 7.21 (d, J=7.1 Hz, 1H), 6.66 (d, J=9.0 Hz, 1H). ¹³C NMR (151 MHz, Methanol-d₄) δ 169.32 (d, J=261.4 Hz), 163.38, 151.46 (d, J=7.9 Hz), 151.30, 141.51, 138.43, 120.20, 111.89 (d, J=17.3 Hz), 107.77 (d, J=20.0 Hz), 105.03. HRMS (ESI-TOF) Calcd for C₁₀H₈FN₂O [M+H]⁺: 191.0621; found: 191.0623.

### Example 6: 6′-chloro-[2,2′-bipyridin]-6(1H)-one (L13)

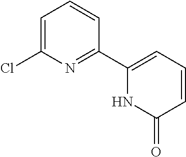 [SMILES: O=c1cccc(-c2cccc(Cl)n2)[nH]1]

Yellow solid, ¹H NMR (600 MHz, Methanol-d₄) δ 8.03 (d, J=7.8 Hz, 1H), 7.95 (t, J=7.9 Hz, 1H), 7.71 (t, J=8.1 Hz, 1H), 7.54 (d, J=7.9 Hz, 1H), 7.19 (d, J=7.0 Hz, 1H), 6.65 (d, J=9.1 Hz, 1H). ¹³C NMR (151 MHz, MeOD) δ 163.47, 150.62, 148.77, 141.49, 140.13, 124.75, 120.09, 118.67, 105.27. HRMS (ESI-TOF) Calcd for C₁₀H₈ClN₂O [M+H]⁺: 207.0325; found: 207.0330.

### Example 7: 4′-(trifluoromethyl)-[2,2′-bipyridin]-6(1H)-one (L14)

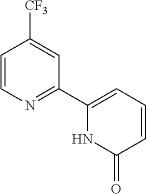 [SMILES: O=c1cccc(-c2cc(C(F)(F)F)ccn2)[nH]1]

Yellow solid, ¹H NMR (600 MHz, Methanol-d₄) δ 8.92 (d, J=5.2 Hz, 1H), 8.36 (s, 1H), 7.75 (dd, J=5.1, 1.5 Hz, 1H), 7.71 (dd, J=9.1, 7.0 Hz, 1H), 7.32 (d, J=7.1 Hz, 1H), 6.67 (d, J=9.1 Hz, 1H). ¹³C NMR (151 MHz, Methanol-d₄) δ 163.42, 150.11, 149.75, 141.40, 139.11 (q, J=34.2 Hz), 122.29 (q, J=272.7 Hz), 120.28, 119.53 (q, J=3.4 Hz), 115.72 (q, J=3.8 Hz), 105.64. HRMS (ESI-TOF) Calcd for C₁₁H₈F₃N₂O [M+H]⁺: 241.0589; found: 241.0591.

### Example 8: 4′-methoxy-[2,2′-bipyridin]-6(1H)-one (L15)

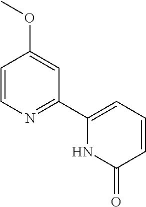

Yellow solid, ¹H NMR (600 MHz, Methanol-d₄) δ 8.50 (d, J=5.8 Hz, 1H), 7.69 (dd, J=9.1, 7.0 Hz, 1H), 7.57 (d, J=2.5 Hz, 1H), 7.16 (d, J=7.1 Hz, 1H), 7.04 (dd, J=5.8, 2.4 Hz, 1H), 6.61 (d, J=9.0 Hz, 1H), 3.95 (s, 3H). ¹³C NMR (151 MHz, MeOD) δ 166.90, 163.51, 149.92, 149.37, 142.36, 141.70, 119.35, 110.30, 106.14, 104.38, 54.49. HRMS (ESI-TOF) Calcd for C₁₁H₁₁N₂O₂[M+H]⁺: 203.0821; found: 203.0819.

### Example 9: 6-(pyrimidin-2-yl)pyridin-2(1H)-one (L16)

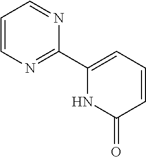 [SMILES: O=c1cccc(-c2ncccn2)[nH]1]

Yellow solid, ¹H NMR (600 MHz, Methanol-d₄) δ 8.90 (d, J=5.0 Hz, 2H), 7.72 (dd, J=9.1, 6.9 Hz, 1H), 7.52 (dd, J=7.0, 1.1 Hz, 1H), 7.48 (t, J=4.9 Hz, 1H), 6.70 (dd, J=9.1, 1.1 Hz, 1H). ¹³C NMR (151 MHz, MeOD) δ 163.35, 157.19, 156.61, 141.46, 140.82, 121.85, 120.82, 106.66. HRMS (ESI-TOF) Calcd for C₉H₈N₃O [M+H]⁺: 174.0667; found: 174.0668.

### Example 10: 6-(isoquinolin-1-yl)pyridin-2(1H)-one (L17)

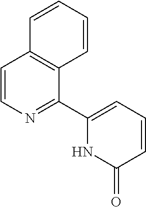 [SMILES: O=c1cccc(-c2nccc3ccccc23)[nH]1]

Yellow solid, ¹H NMR (600 MHz, Methanol-d₄) δ 8.36 (d, J=5.7 Hz, 1H), 7.98 (d, J=8.5 Hz, 1H), 7.83 (d, J=8.3 Hz, 1H), 7.71 (d, J=5.7 Hz, 1H), 7.62 (t, J=7.6 Hz, 1H), 7.57-7.48 (m, 2H), 6.54 (d, J=6.8 Hz, 1H), 6.48 (d, J=9.2 Hz, 1H). ¹³C NMR (151 MHz, MeOD) δ 164.57, 152.16, 143.85, 141.54, 141.19, 137.18, 130.93, 128.44, 127.21, 126.26, 125.55, 122.27, 119.63, 109.37. HRMS (ESI-TOF) Calcd for C₁₄H₁₁N₂O [M+H]⁺: 223.0871; found: 223.0877.

### Example 11: 6-(isoquinolin-3-yl)pyridin-2(1H)-one (L18)

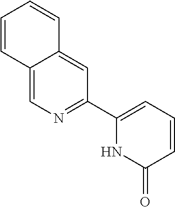 [SMILES: O=c1cccc(-c2cc3ccccc3cn2)[nH]1]

Yellow solid, ¹H NMR (600 MHz, Methanol-d₄) δ 9.29 (s, 1H), 8.42 (s, 1H), 8.09 (d, J=8.2 Hz, 1H), 7.99 (d, J=8.2 Hz, 1H), 7.80 (ddd, J=8.1, 6.8, 1.3 Hz, 1H), 7.73-7.68 (m, 2H), 7.22 (d, J=7.1 Hz, 1H), 6.58 (d, J=8.9 Hz, 1H). ¹³C NMR (151 MHz, MeOD) δ 164.04, 152.20, 143.37, 142.39, 141.53, 136.16, 131.31, 128.94, 128.69, 127.62, 127.17, 118.88, 117.73, 104.21. HRMS (ESI-TOF) Calcd for C₁₄H₁₁N₂O [M+H]⁺: 223.0871; found: 223.0877.

### Example 12: 5-methoxy-[2,2′-bipyridin]-6(1H)-one (L19)

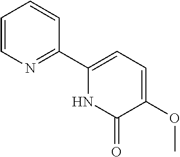

Yellow solid, ¹H NMR (600 MHz, Methanol-d₄) δ 8.65-8.58 (m, 1H), 7.96-7.83 (m, 2H), 7.40-7.29 (m, 1H), 7.12-7.01 (m, 2H), 3.88 (s, 3H). ¹³C NMR (151 MHz, MeOD) δ 157.94, 150.20, 148.40, 147.95, 136.94, 132.69, 123.00, 118.66, 114.16, 104.18, 54.73. HRMS (ESI-TOF) Calcd for C₁₁H₁₁N₂O₂[M+H]⁺: 203.0821; found: 203.0824.

### Scheme for Preparing Bidentate Pyridine-Pyridone Ligands L20-L39 of Formula (L-2)

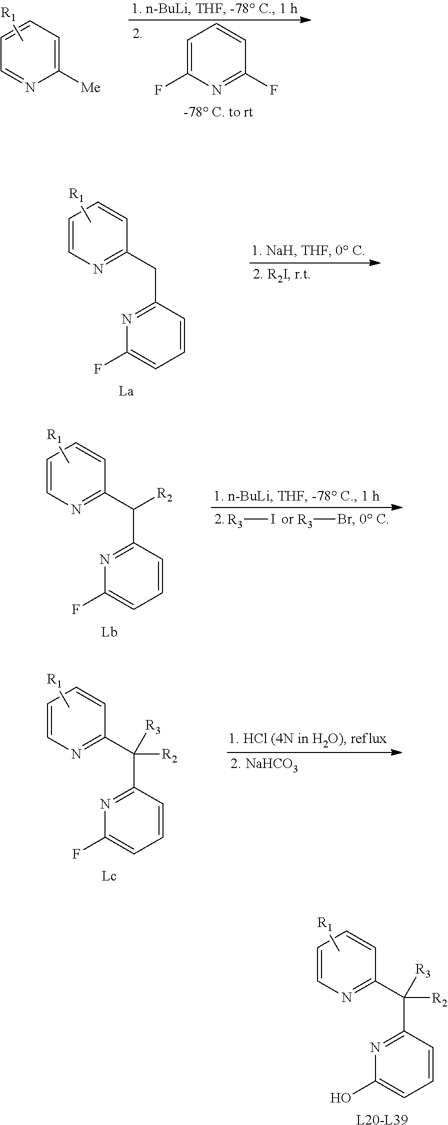

### Synthesis of Formula (L-2) Intermediate Compounds

Synthesis of La. To a solution of R₁ substituted 2-picoline (50 mmol, 1.0 equiv.) in anhydrous THF (80 mL) at −78° C. was added n-butyllithium (2.5 M in hexanes, 50 mmol, 20.0 mL, 1.0 equiv.) dropwise. The resulting solution was stirred for 1 hour at −78° C. before 2,6-difluoropyridine (5.75 g, 50 mmol, 1.0 equiv.) was added in a single batch. The reaction mixture was warmed to room temperature gradually and stirred for 3 hours. Upon completion, saturated aqueous NH₄Cl solution was added and extracted with DCM three times. The combined extracts were washed with brine, dried over anhydrous Na₂SO₄, and concentrated under vacuum. Flash chromatography (eluent: ethyl acetate/hexanes=1/10 to 1/4) gave La (40%˜50% yield).

Synthesis of Lb and Lc. To a solution of La (20 mmol, 1.0 equiv) in anhydrous THF (50 mL) at 0° C. was added NaH (60% in mineral oil, 1.20 g, 30 mmol, 1.5 equiv.). The resulting solution was stirred for 1 h at 0° C. before alkyl iodide R₂—I (30 mmol, 1.5 equiv.) was added dropwise. The reaction mixture was warmed to room temperature gradually and stirred for 5 hours. Upon completion, saturated aqueous NH₄Cl solution was added and extracted with DCM three times. The combined extracts were washed with brine, dried over anhydrous Na₂SO₄, and concentrated under vacuum to give Lb, which was used for next step without purification.

To a solution of Lb in anhydrous THF (50 mL) at 0° C. was added n-butyllithium (2.5 M in hexanes, 30 mmol, 12 mL, 1.5 equiv.) dropwise. The resulting solution was stirred for 1 hour at −78° C. before alkyl iodide or alkyl bromide (R₃—I or R₃—Br) (30 mmol, 1.5 equiv.) was added. The reaction mixture was warmed to 0° C. and stirred for 3 hours. Upon completion, the reaction mixture was cooled to room temperature. Water was added to the mixture and extracted three times with dichloromethane. The combined organic layers were washed with brine, dried over anhydrous Na₂SO₄, and concentrated under vacuum. The residue was purified by flash chromatography (eluent: ethyl acetate/hexanes=1/6 to 1/3), affording the pure product (Lc).

### Synthesis of Ligands L20-L39

General procedure: a suspension of Lc in 4N HCl in water (15 mL) was refluxed for 12 hours. Upon completion (determined by TLC monitoring), the reaction mixture was cooled to room temperature and quenched with saturated aqueous NaHCO₃ solution to neutral pH. The aqueous phase was extracted three times with dichloromethane. The combined organic layers were washed with brine, dried over anhydrous Na₂SO₄, and concentrated under vacuum. The residue was purified by flash chromatography (eluent: ethyl acetate/methanol=100/1 to 10/1) to give the pure product (L20-L39).

### Example 13: Optimized Procedure for the Preparation of Ligands L33 and L39

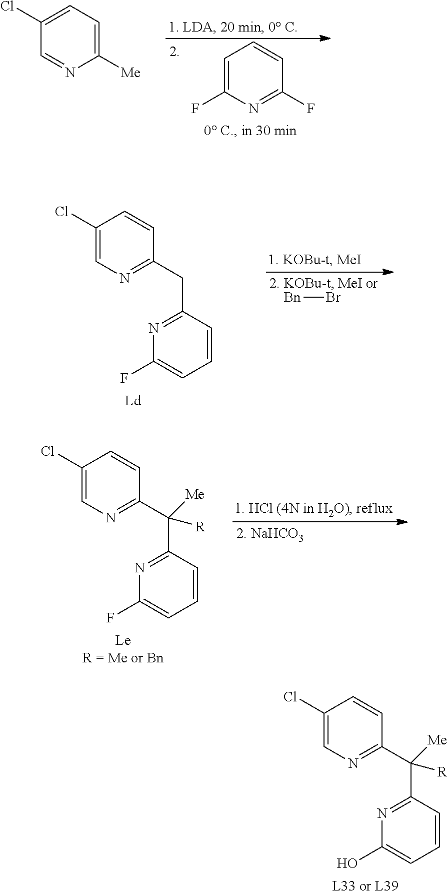

Synthesis of Ld. To a solution of 5-chloro-2-methylpyridine (6.34 g, 50 mmol, 1.0 equiv.) in anhydrous THE (100 mL) at 0° C. was added freshly prepared LDA (50 mmol, 1.0 equiv.) slowly. The resulting solution was stirred for 20 minutes at 0° C. before 2,6-difluoropyridine (5.75 g, 50 mmol, 1.0 equiv.) was added in a single batch. The reaction mixture was kept at 0° C. and stirred for 30 minutes. Upon completion, saturated aqueous NH₄Cl solution was added and extracted with DCM three times. The combined extracts were washed with brine, dried over anhydrous Na₂SO₄, and concentrated under vacuum. Flash chromatography (eluent: ethyl acetate/hexanes=1/10) gave Ld (8.0 g, 72% yield).

Synthesis of Le. To a solution of Ld (4.4 g, 20 mmol, 1.0 equiv) in anhydrous THE (50 mL) at room temperature was added potassium tert-butoxide (2.24 g, 20 mmol, 1.0 equiv.) under N₂. The resulting solution was stirred for 30 minutes before methyl iodide (20 mmol, 1.0 equiv.) was added dropwise. The reaction mixture was stirred for 1 hour. Next, potassium tert-butoxide (2.24 g, 20 mmol, 1.0 equiv.) was added for a second time under N₂. The resulting solution was stirred for 30 minutes before methyl iodide or benzyl bromide (20 mmol, 1.0 equiv.) was added dropwise. The reaction mixture was stirred for another 1 hour. Upon completion, saturated aqueous NH₄Cl solution was added and extracted with DCM three times. The combined extracts were washed with brine, dried over anhydrous Na₂SO₄, and concentrated under vacuum to give Le, which was used for next step without purification.

Synthesis of L33 or L39. A suspension of Le in 4N HCl in water (15 mL) was refluxed for 12 hours. Upon completion, the reaction mixture was cooled to room temperature and quenched with saturated aqueous NaHCO₃ solution to neutral pH. The aqueous phase was extracted three times with dichloromethane. The combined organic layers were washed with brine, dried over anhydrous Na₂SO₄, and concentrated under vacuum. The residue was purified by flash chromatography (eluent: ethyl acetate/methanol=100/1 to 10/1) to give the pure product (L33 or L39).

### Example 14: 6-(2-(5-methylpyridin-2-yl)-1-phenylpropan-2-yl)pyridin-2(1H)-one (L6)

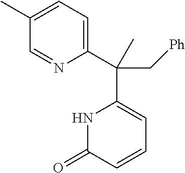 [SMILES: Cc1ccc(C(C)(Cc2ccccc2)c2cccc(=O)[nH]2)nc1]

Yellow solid, ¹H NMR (600 MHz, CDCl₃) δ 11.26 (brs, 1H), 8.59 (s, 1H), 7.45 (dd, J=8.2, 2.3 Hz, 1H), 7.24 (dd, J=9.2, 7.0 Hz, 1H), 7.16-7.10 (m, 4H), 6.75 (d, J=7.8 Hz, 2H), 6.40 (d, J=9.2 Hz, 1H), 5.96 (d, J=6.9 Hz, 1H), 3.45-3.34 (m, 2H), 2.35 (s, 3H), 1.57 (s, 3H). ¹³C NMR (151 MHz, CDCl₃) δ 163.79, 159.32, 151.90, 149.51, 140.67, 137.87, 136.95, 132.14, 130.33, 127.95, 126.75, 120.64, 118.77, 102.91, 48.91, 46.48, 21.12, 18.15. HRMS (ESI-TOF) Calcd for C₂₀H₂₁N₂O [M+H]⁺: 305.1654; found: 305.1657.

### Example 15: 6-(1-Cyclohexyl-1-(5-methylpyridin-2-yl)ethyl)pyridin-2(11)-one (L7)

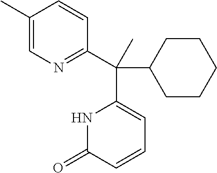 [SMILES: Cc1ccc(C(C)(c2cccc(=O)[nH]2)C2CCCCC2)nc1]

Yellow solid, ¹H NMR (600 MHz, CDCl₃) δ 11.59 (brs, 1H), 8.52 (d, J=2.4 Hz, 1H), 7.44 (dd, J=8.1, 2.4 Hz, 1H), 7.29-7.20 (m, 2H), 6.34 (dd, J=9.1, 2.0 Hz, 1H), 6.10 (dd, J=7.1, 1.8 Hz, 1H), 2.57-2.52 (m, 1H), 2.31 (s, 3H), 1.70-1.60 (m, 3H), 1.58 (s, 3H), 1.26-1.18 (m, 2H), 1.16-0.95 (m, 3H), 0.91-0.88 (m, 2H). ¹³C NMR (151 MHz, CDCl₃) δ 163.41, 158.97, 151.76, 148.98, 139.72, 136.90, 130.95, 120.14, 117.66, 102.47, 48.03, 47.76, 27.46, 27.07, 26.41, 26.29, 26.06, 17.45, 14.87. HRMS (ESI-TOF) Calcd for C₁₉H₂₅N₂O [M+H]⁺: 297.1967; found: 297.1970.

### Example 16: 6-(1-(quinolin-2-yl)ethyl)pyridin-2-ol (L20)

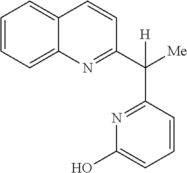 [SMILES: CC(c1cccc(O)n1)c1ccc2ccccc2n1]

Yellow solid, ¹H NMR (600 MHz, Chloroform-d) δ 8.16 (dd, J=8.5, 1.0 Hz, 1H), 8.12 (dd, J=8.5, 0.8 Hz, 1H), 7.79 (dd, J=8.1, 1.4 Hz, 1H), 7.73 (ddd, J=8.4, 6.9, 1.5 Hz, 1H), 7.53 (ddd, J=8.1, 6.9, 1.2 Hz, 1H), 7.35 (d, J=8.4 Hz, 1H), 7.32 (dd, J=9.2, 6.8 Hz, 1H), 6.40 (dd, J=9.2, 1.0 Hz, 1H), 6.15 (dd, J=6.8, 1.1 Hz, 1H), 4.22 (q, J=7.2 Hz, 1H), 1.77 (d, J=7.2 Hz, 3H); ¹³C NMR (151 MHz, CDCl₃) δ 164.08, 160.70, 150.19, 147.66, 141.18, 137.47, 129.97, 129.39, 127.49, 127.16, 126.67, 120.61, 118.60, 103.86, 45.04, 21.27. HRMS (ESI-TOF) m/z Calcd for C₁₆H₁₅N₂O [M+H]⁺ 251.1184, found 251.1189.

### Example 17: 6-(2-(quinolin-2-yl)propan-2-yl)pyridin-2-ol (L5)

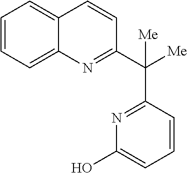 [SMILES: CC(C)(c1cccc(O)n1)c1ccc2ccccc2n1]

Yellow solid, ¹H NMR (600 MHz, Chloroform-d) δ 8.16 (dq, J=8.5, 0.9 Hz, 1H), 8.10 (dd, J=8.6, 0.9 Hz, 1H), 7.78 (dd, J=8.2, 1.4 Hz, 1H), 7.73 (ddd, J=8.4, 6.9, 1.4 Hz, 1H), 7.54 (ddd, J=8.1, 6.9, 1.2 Hz, 1H), 7.38 (d, J=8.6 Hz, 1H), 7.31 (dd, J=9.2, 7.0 Hz, 1H), 6.32 (dd, J=9.2, 1.0 Hz, 1H), 6.22 (dd, J=7.0, 1.0 Hz, 1H), 1.85 (s, 6H); ¹³C NMR (151 MHz, CDCl₃) δ 163.62, 163.11, 153.35, 147.22, 140.82, 137.25, 129.93, 129.58, 127.30, 126.83, 126.79, 118.54, 118.31, 101.73, 44.23, 27.52. HRMS (ESI-TOF) m/z Calcd for C₁₇H₁₇N₂O⁺ [M+H]⁺ 265.1341, found 265.1341.

### Example 18: 6-(2-(quinolin-2-yl)butan-2-yl)pyridin-2-ol (L21)

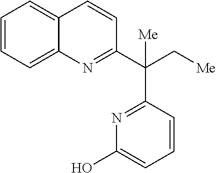 [SMILES: CCC(C)(c1cccc(O)n1)c1ccc2ccccc2n1]

Yellow solid, ¹H NMR (600 MHz, Chloroform-d) δ 8.19 (dd, J=8.5, 2.7 Hz, 1H), 8.12 (dd, J=8.7, 2.6 Hz, 1H), 7.79 (dt, J=8.1, 2.0 Hz, 1H), 7.76-7.71 (m, 1H), 7.55 (tq, J=6.9, 2.5, 1.9 Hz, 1H), 7.40 (dd, J=8.6, 2.6 Hz, 1H), 7.33 (ddd, J=9.5, 7.1, 2.6 Hz, 1H), 6.36 (dd, J=9.3, 2.7 Hz, 1H), 6.23 (dd, J=7.2, 2.6 Hz, 1H), 2.33 (qd, J=7.5, 2.5 Hz, 2H), 1.80 (s, 3H), 0.80 (td, J=7.6, 2.5 Hz, 3H); ¹³C NMR (151 MHz, CDCl₃) δ 163.57, 162.55, 152.27, 147.18, 140.62, 137.25, 129.99, 129.61, 127.29, 126.90, 126.75, 118.77, 118.56, 102.64, 47.52, 34.17, 21.53, 8.89. HRMS (ESI-TOF) m/z Calcd for C₁₈H₁₉N₂O [M+H]⁺ 279.1497, found 279.1499.

### Example 19: 6-(1-phenyl-2-(quinolin-2-yl)propan-2-yl)pyridin-2-ol (L22)

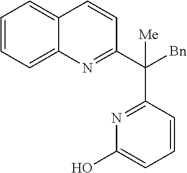 [SMILES: CC(Cc1ccccc1)(c1cccc(O)n1)c1ccc2ccccc2n1]

Yellow solid, ¹H NMR (600 MHz, Chloroform-d) δ 8.30 (dq, J=8.5, 0.9 Hz, 1H), 8.14 (dd, J=8.7, 0.9 Hz, 1H), 7.85-7.78 (m, 2H), 7.59 (ddd, J=8.1, 6.9, 1.2 Hz, 1H), 7.39 (d, J=8.6 Hz, 1H), 7.26-7.24 (m, 1H), 7.17-7.11 (m, 3H), 6.84-6.81 (m, 2H), 6.40 (dd, J=9.1, 1.0 Hz, 1H), 6.01 (dd, J=7.0, 0.9 Hz, 1H), 3.65 (d, J=13.6 Hz, 1H), 3.58 (d, J=13.5 Hz, 1H), 1.71 (s, 3H); ¹³C NMR (151 MHz, CDCl₃) δ 163.53, 162.33, 151.22, 146.98, 140.50, 137.51, 136.89, 130.31, 130.26, 129.59, 127.90, 127.37, 127.11, 126.86, 118.88, 118.86, 103.26, 48.18, 47.73, 21.52. HRMS (ESI-TOF) m/z Calcd for C₂₃H₂₁N₂O⁺ [M+H]⁺ 341.1654, found 341.1649.

### Example 20: 6-(1-phenyl-2-(quinolin-2-yl)butan-2-yl)pyridin-2-ol (L23)

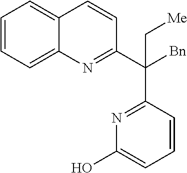 [SMILES: CCC(Cc1ccccc1)(c1cccc(O)n1)c1ccc2ccccc2n1]

Yellow solid, ¹H NMR (600 MHz, Chloroform-d) δ 8.16 (dq, J=9.2, 0.8 Hz, 1H), 8.11 (dd, J=8.6, 0.8 Hz, 1H), 7.81 (dd, J=8.1, 1.4 Hz, 1H), 7.76 (ddd, J=8.4, 6.9, 1.5 Hz, 1H), 7.59 (ddd, J=8.1, 6.9, 1.2 Hz, 1H), 7.29 (dd, J=9.2, 7.0 Hz, 1H), 7.22-7.27 (m, 1H), 7.17-7.11 (m, 3H), 6.81-6.77 (m, 2H), 6.39 (dd, J=9.2, 0.9 Hz, 1H), 6.04 (dd, J=7.1, 1.0 Hz, 1H), 3.71 (d, J=14.1 Hz, 1H), 3.60 (d, J=14.1 Hz, 1H), 2.13 (dp, J=21.5, 7.2 Hz, 2H), 0.86 (t, J=7.4 Hz, 3H); ¹³C NMR (151 MHz, CDCl₃) δ 163.36, 161.41, 151.45, 147.14, 140.62, 137.05, 130.02, 129.92, 129.72, 127.95, 127.38, 127.09, 126.83, 126.64, 120.10, 118.61, 104.68, 53.21, 41.31, 26.64, 8.54. HRMS (ESI-TOF) m/z Calcd for C₂₄H₂₃N₂O⁺ [M+H]⁺ 355.1810, found 355.1812.

### Example 21: 6-(1,3-diphenyl-2-(quinolin-2-yl)propan-2-yl)pyridin-2-ol (L24)

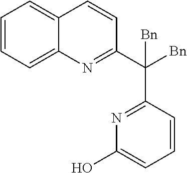 [SMILES: Oc1cccc(C(Cc2ccccc2)(Cc2ccccc2)c2ccc3ccccc3n2)n1]

Yellow solid, ¹H NMR (600 MHz, Methanol-d₄) δ 8.99 (d, J=8.8 Hz, 1H), 8.88 (d, J=8.8 Hz, 1H), 8.30 (d, J=7.2 Hz, 1H), 8.23-8.21 (m, 1H), 8.18 (d, J=8.7 Hz, 1H), 8.12 (d, J=2.7 Hz, 1H), 8.02-8.00 (m, 1H), 7.98-7.96 (m, 1H), 7.89 (td, J=7.5, 6.9, 1.0 Hz, 1H), 7.71 (d, J=7.9 Hz, 1H), 7.12 (d, J=1.9 Hz, 3H), 7.00-7.05 (m, 2H), 6.91 (d, J=6.9 Hz, 2H), 6.73 (s, 2H), 3.87 (d, J=13.7 Hz, 2H), 3.78 (d, J=13.7 Hz, 2H); ¹³C NMR (151 MHz, CDCl₃) δ 163.99, 163.64, 163.02, 162.94, 162.05, 153.05, 142.05, 142.00, 141.13, 118.75, 117.95, 117.93, 108.37, 108.13, 102.61, 44.16, 27.31. HRMS (ESI-TOF) m/z Calcd for C₂₉H₂₅N₂O⁺ [M+H]⁺ 417.1967, found 417.1961.

### Example 22: 6-(1-(3,5-dibromophenyl)-2-(quinolin-2-yl)propan-2-yl)pyridin-2-ol (L25)

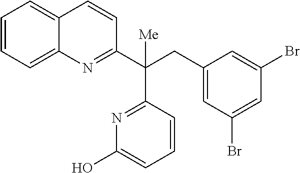 [SMILES: CC(Cc1cc(Br)cc(Br)c1)(c1cccc(O)n1)c1ccc2ccccc2n1]

Yellow solid, ¹H NMR (600 MHz, Chloroform-d) δ 8.28 (dd, J=8.4, 1.1 Hz, 1H), 8.15 (dd, J=8.8, 0.8 Hz, 1H), 7.84-7.79 (m, 2H), 7.60 (ddd, J=8.1, 6.9, 1.2 Hz, 1H), 7.38 (d, J=8.6 Hz, 1H), 7.29 (ddd, J=8.3, 2.2, 1.2 Hz, 1H), 7.03 (t, J=1.8 Hz, 1H), 6.97 (t, J=7.8 Hz, 1H), 6.70 (dt, J=7.9, 1.3 Hz, 1H), 6.39 (dd, J=9.2, 0.9 Hz, 1H), 6.00 (dd, J=7.0, 1.0 Hz, 1H), 3.63 (d, J=13.5 Hz, 1H), 3.53 (d, J=13.5 Hz, 1H), 1.70 (s, 3H); ¹³C NMR (151 MHz, CDCl₃) δ 163.52, 161.95, 150.73, 146.96, 140.44, 139.28, 137.64, 133.39, 130.35, 129.82, 129.51, 129.43, 128.79, 127.40, 127.21, 126.89, 121.92, 119.10, 118.82, 103.44, 47.72, 47.50, 21.56. HRMS (ESI-TOF) m/z Calcd for C₂₃H₁₉Br₂N₂O⁺ [M+H]⁺ 496.9864, found 496.9872.

### Example 23: 6-(1-(3,5-di-tert-butylphenyl)-2-(quinolin-2-yl)propan-2-yl)pyridin-2-ol (L26)

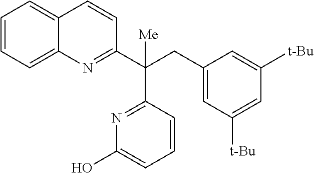 [SMILES: CC(C)(C)c1cc(CC(C)(c2cccc(O)n2)c2ccc3ccccc3n2)cc(C(C)(C)C)c1]

Yellow solid, ¹H NMR (600 MHz, Chloroform-d) δ 8.32 (dt, J=8.5, 1.0 Hz, 1H), 8.14-8.10 (m, 1H), 7.83-7.76 (m, 2H), 7.58 (ddd, J=8.2, 6.9, 1.3 Hz, 1H), 7.38 (dd, J=8.6, 1.2 Hz, 1H), 7.29-7.26 (m, 1H), 7.17 (t, J=1.8 Hz, 1H), 6.63 (d, J=1.8 Hz, 2H), 6.41 (dd, J=9.1, 1.0 Hz, 1H), 6.03 (dd, J=7.1, 1.0 Hz, 1H), 3.60 (d, J=13.4 Hz, 1H), 3.54 (d, J=13.4 Hz, 1H), 1.70 (s, 3H), 1.13 (s, 18H); ¹³C NMR (151 MHz, CDCl₃) δ 163.59, 162.53, 151.59, 150.05, 146.97, 140.60, 137.35, 135.64, 130.22, 129.54, 127.27, 127.06, 126.82, 124.63, 120.34, 118.94, 118.71, 103.30, 49.09, 47.74, 34.54, 31.32, 21.48. HRMS (ESI-TOF) m/z Calcd for C₃₁H₃₇N₂O⁺ [M+H]⁺ 453.2906, found 453.2906.

### Example 24: 6-(4-(tert-butyl)-1-(quinolin-2-yl)cyclohexyl)pyridin-2-ol

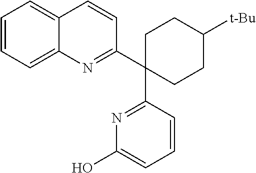 [SMILES: CC(C)(C)C1CCC(c2cccc(O)n2)(c2ccc3ccccc3n2)CC1]

Yellow solid, ¹H NMR (600 MHz, Chloroform-d) δ 8.06 (s, 2H), 7.74 (d, J=8.0 Hz, 1H), 7.65-7.69 (m, 1H), 7.62-7.52 (m, 1H), 7.49 (t, J=7.2 Hz, 1H), 7.35-7.39 (m, 1H), 6.54 (m, 1H), 2.09-1.99 (m, 2H), 1.86 (s, 2H), 1.24 (d, J=17.9 Hz, 4H), 0.83 (s, 9H); ¹³C NMR (151 MHz, CDCl₃) δ 163.45, 147.31, 137.15, 129.74, 129.45, 127.27, 126.79, 126.60, 117.89, 47.74, 35.01, 32.34, 29.73, 27.39, 23.72. HRMS (ESI-TOF) m/z Calcd for C₂₄H₂₉N₂O⁺ [M+H]⁺ 361.2280, found 361.2280.

### Example 25: 6-(8-methyl-5,6,7,8-tetrahydroquinolin-8-yl)pyridin-2-ol (L28)

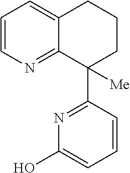 [SMILES: CC1(c2cccc(O)n2)CCCc2cccnc21]

Yellow solid, ¹H NMR (600 MHz, Chloroform-d) δ 8.55-8.49 (m, 1H), 7.46-7.42 (m, 1H), 7.33 (dd, J=9.1, 7.0 Hz, 1H), 7.14 (dd, J=7.7, 4.7 Hz, 1H), 6.37 (dd, J=9.1, 0.9 Hz, 1H), 6.14 (dd, J=7.1, 1.0 Hz, 1H), 2.90-2.76 (m, 2H), 2.51-2.42 (m, 1H), 1.96-1.84 (m, 3H), 1.68 (s, 3H); ¹³C NMR (151 MHz, CDCl₃) δ 163.87, 158.25, 153.47, 147.29, 140.85, 137.87, 132.75, 122.17, 118.10, 101.67, 42.60, 35.16, 31.29, 29.61, 19.16. HRMS (ESI-TOF) m/z Calcd for C₁₅H₁₇N₂O⁺ [M+H]⁺ 241.1341, found 241.1345.

### Example 26: 6-(1-(quinolin-2-yl)cyclopentyl)pyridin-2-ol (L29)

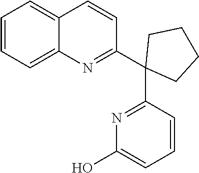 [SMILES: Oc1cccc(C2(c3ccc4ccccc4n3)CCCC2)n1]

Yellow solid, ¹H NMR (600 MHz, Chloroform-d) δ 8.14 (dd, J=8.4, 1.2 Hz, 1H), 8.08 (d, J=8.6 Hz, 1H), 7.77 (dd, J=8.1, 1.4 Hz, 1H), 7.73 (ddd, J=8.4, 6.9, 1.4 Hz, 1H), 7.54 (ddd, J=8.1, 6.9, 1.2 Hz, 1H), 7.37 (d, J=8.6 Hz, 1H), 7.31 (dd, J=9.2, 7.0 Hz, 1H), 6.32 (dd, J=9.2, 1.0 Hz, 1H), 6.25 (dd, J=7.0, 1.0 Hz, 1H), 2.71 (dddd, J=12.8, 7.3, 5.5, 2.2 Hz, 2H), 2.39-2.25 (m, 2H), 1.85-1.78 (m, 2H), 1.77-1.69 (m, 2H); ¹³C NMR (151 MHz, CDCl₃) δ 161.91, 151.60, 147.37, 140.78, 137.24, 129.85, 129.66, 127.30, 126.83, 126.83, 118.88, 101.90, 57.13, 36.32, 23.04. HRMS (ESI-TOF) m/z Calcd for C₁₉H₁₉N₂O⁺ [M+H]⁺ 291.1497, found 291.1501.

### Example 27: 6-(1-hydroxy-2-(naphthalen-2-yl)-1-(quinolin-2-yl)ethyl)pyridin-2-ol (L30)

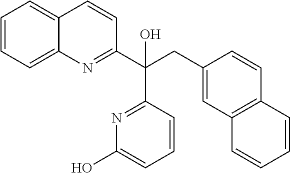 [SMILES: Oc1cccc(C(O)(Cc2ccc3ccccc3c2)c2ccc3ccccc3n2)n1]

Yellow solid, ¹H NMR (600 MHz, Methanol-d₄) δ 8.26 (dd, J=8.7, 0.8 Hz, 1H), 8.20-8.17 (m, 1H), 8.00 (d, J=8.5 Hz, 1H), 7.90-7.87 (m, 1H), 7.85 (d, J=8.5 Hz, 1H), 7.78 (ddd, J=8.4, 6.9, 1.4 Hz, 1H), 7.74-7.71 (m, 1H), 7.66 (d, J=8.1 Hz, 1H), 7.60 (ddd, J=8.1, 6.9, 1.2 Hz, 1H), 7.47 (dd, J=9.0, 7.1 Hz, 1H), 7.32 (ddd, J=8.1, 6.8, 1.2 Hz, 1H), 7.29-7.25 (m, 1H), 7.20 (dd, J=8.1, 7.1 Hz, 1H), 7.15 (dd, J=7.2, 1.3 Hz, 1H), 6.70 (d, J=7.1 Hz, 1H), 6.36 (dd, J=9.0, 1.0 Hz, 1H), 4.21 (d, J=14.3 Hz, 1H), 4.04 (d, J=14.3 Hz, 1H); ¹³C NMR (151 MHz, MeOD) δ 163.81, 161.74, 146.05, 142.06, 137.61, 133.67, 133.12, 131.53, 129.98, 128.89, 128.67, 128.03, 127.43, 127.38, 127.35, 126.94, 125.11, 124.86, 124.48, 124.28, 118.30, 117.32, 105.23, 76.50, 43.96. HRMS (ESI-TOF) m/z Calcd for C₂₆H₂₁N₂O₂⁺ [M+H]⁺ 393.1603, found 393.1609.

### Example 28: 3,5-diiodo-6-(2-(quinolin-2-yl)propan-2-yl)pyridin-2-ol (L31)

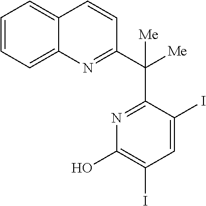 [SMILES: CC(C)(c1ccc2ccccc2n1)c1nc(O)c(I)cc1I]

Yellow solid, ¹H NMR (600 MHz, Chloroform-d) δ 8.24 (s, 1H), 8.11 (dd, J=8.6, 0.8 Hz, 1H), 8.02 (dd, J=8.5, 1.0 Hz, 1H), 7.81 (dd, J=8.2, 1.4 Hz, 1H), 7.71 (ddd, J=8.4, 6.9, 1.4 Hz, 1H), 7.54 (ddd, J=8.1, 6.9, 1.2 Hz, 1H), 7.23 (d, J=8.6 Hz, 1H), 1.91 (s, 6H); ¹³C NMR (151 MHz, CDCl₃) δ 163.15, 160.57, 159.96, 147.54, 136.91, 129.67, 129.48, 127.46, 126.74, 126.58, 119.38, 49.12, 26.66. HRMS (ESI-TOF) m/z Calcd for C₁₇H₁₅I₂N₂O⁺ [M+H]⁺ 516.9274, found 516.9271.

### Example 29: 3-chloro-6-(2-(quinolin-2-yl)propan-2-yl)pyridin-2-ol (L32)

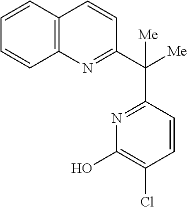 [SMILES: CC(C)(c1ccc(Cl)c(O)n1)c1ccc2ccccc2n1]

Yellow solid, ¹H NMR (600 MHz, Chloroform-d) δ 8.09 (d, J=8.6 Hz, 1H), 8.05-8.00 (m, 1H), 7.79 (dd, J=8.2, 1.5 Hz, 1H), 7.70 (ddd, J=8.4, 6.9, 1.5 Hz, 1H), 7.52 (ddd, J=8.1, 6.8, 1.2 Hz, 1H), 7.27 (d, J=9.5 Hz, 1H), 7.25 (s, 1H), 6.48 (d, J=9.5 Hz, 1H), 1.88 (s, 6H); ¹³C NMR (151 MHz, CDCl₃) δ 177.28, 175.91, 163.77, 162.03, 147.52, 144.77, 136.77, 129.56, 129.45, 127.41, 126.70, 126.40, 118.55, 118.38, 47.37, 26.21. HRMS (ESI-TOF) m/z Calcd for C₁₇H₁₆ClN₂O⁺ [M+H]⁺ 299.0951, found 299.0950.

### Example 30: 6-(2-(5-chloropyridin-2-yl)propan-2-yl)pyridin-2-ol (L33)

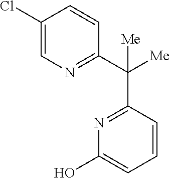 [SMILES: CC(C)(c1ccc(Cl)cn1)c1cccc(O)n1]

Yellow solid, ¹H NMR (600 MHz, Chloroform-d) δ 8.61-8.56 (m, 1H), 7.63 (ddd, J=8.5, 2.5, 0.8 Hz, 1H), 7.33 (ddd, J=9.3, 7.0, 0.8 Hz, 1H), 7.26 (d, J=5.5 Hz, 1H), 6.37 (dt, J=9.2, 0.9 Hz, 1H), 6.17 (dd, J=7.0, 1.0 Hz, 1H), 1.72 (s, 6H); ¹³C NMR (151 MHz, CDCl₃) δ 163.62, 161.51, 152.82, 148.07, 140.87, 136.77, 130.81, 121.11, 118.70, 101.72, 43.24, 27.53. HRMS (ESI-TOF) m/z Calcd for C₁₃H₁₄ClN₂O⁺ [M+H]⁺ 249.0795, found 249.0797.

### Example 31: 6-(2-(6-fluoropyridin-2-yl)propan-2-yl)pyridin-2-ol (L34)

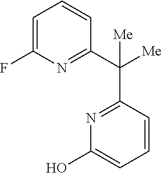 [SMILES: CC(C)(c1cccc(O)n1)c1cccc(F)n1]

Yellow solid, ¹H NMR (600 MHz, Chloroform-d) δ 7.74 (q, J=8.0 Hz, 1H), 7.35 (dd, J=9.2, 7.0 Hz, 1H), 7.13 (dd, J=7.6, 2.4 Hz, 1H), 6.81 (dd, J=8.2, 3.0 Hz, 1H), 6.37 (dd, J=9.2, 1.0 Hz, 1H), 6.18 (dd, J=7.0, 1.0 Hz, 1H), 1.72 (s, 6H); ¹³C NMR (151 MHz, CDCl₃) δ 163.87, 162.86 (d, J=12.08 Hz), 162.72 (d, J=241.60 Hz), 152.93, 141.91 (d, J=7.55 Hz), 141.01, 118.63, 117.82 (d, J=4.53 Hz), 108.13 (d, J=37.75 Hz), 102.49, 44.03, 27.19. HRMS (ESI-TOF) m/z Calcd for C₁₃H₁₄FN₂O⁺ [M+H]⁺ 233.1090, found 233.1092.

### Example 32: 6-(2-(3-methylpyridin-2-yl)propan-2-yl)pyridin-2-ol (L35)

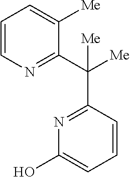 [SMILES: Cc1cccnc1C(C)(C)c1cccc(O)n1]

Yellow solid, ¹H NMR (600 MHz, Chloroform-d) δ 8.46 (dd, J=4.7, 1.9 Hz, 1H), 7.47-7.37 (m, 2H), 7.17 (dd, J=7.6, 4.7 Hz, 1H), 6.39 (d, J=9.2 Hz, 1H), 6.24 (d, J=7.0 Hz, 1H), 2.04 (s, 3H), 1.71 (s, 6H); ¹³C NMR (151 MHz, CDCl₃) δ 159.90, 154.94, 146.23, 141.38, 140.46, 132.10, 122.89, 101.96, 46.25, 27.74, 19.57. HRMS (ESI-TOF) m z Calcd for C₁₄H₁₇N₂O⁺ [M+H]⁺ 229.1341, found 229.1344.

### Example 33: 6-(2-(6-methylpyridin-2-yl)propan-2-yl)pyridin-2-ol (L36)

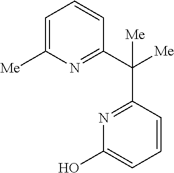 [SMILES: Cc1cccc(C(C)(C)c2cccc(O)n2)n1]

Yellow solid, ¹H NMR (600 MHz, Chloroform-d) δ 7.54 (t, J=7.8 Hz, 1H), 7.32 (dd, J=9.1, 7.0 Hz, 1H), 7.12 (d, J=7.9 Hz, 1H), 7.05 (d, J=7.7 Hz, 1H), 6.37 (d, J=9.1 Hz, 1H), 6.17 (d, J=7.0 Hz, 1H), 2.62 (s, 3H), 1.72 (s, 6H); ¹³C NMR (151 MHz, CDCl₃) δ 163.61, 162.34, 158.11, 153.55, 140.82, 137.32, 121.77, 118.37, 116.83, 101.18, 42.58, 27.72, 24.64. HRMS (ESI-TOF) m/z Calcd for C₁₄H₁₇N₂O⁺ [M+H]⁺ 229.1341, found 229.1340.

### Example 34: 6-(2-(4-methylpyridin-2-yl)propan-2-yl)pyridin-2-ol (L37)

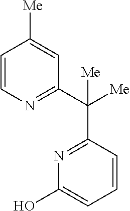 [SMILES: Cc1ccnc(C(C)(C)c2cccc(O)n2)c1]

Yellow solid, ¹H NMR (600 MHz, Chloroform-d) δ 8.49 (dd, J=5.0, 0.8 Hz, 1H), 7.33 (dd, J=9.2, 7.0 Hz, 1H), 7.13 (s, 1H), 7.02 (ddd, J=5.0, 1.6, 0.9 Hz, 1H), 6.37 (dd, J=9.2, 1.0 Hz, 1H), 6.19 (dd, J=7.0, 1.0 Hz, 1H), 2.34 (s, 3H), 1.72 (s, 6H); ¹³C NMR (151 MHz, CDCl₃) δ 163.58, 162.93, 153.55, 148.92, 148.41, 140.87, 123.12, 120.99, 118.47, 101.29, 42.93, 27.67, 21.34. HRMS (ESI-TOF) m/z Calcd for C₁₄H₁₇N₂O⁺ [M+H]⁺ 229.1341, found 229.1339.

### Example 35: 6-(2-(4-methoxypyridin-2-yl)propan-2-yl)pyridin-2-ol (L38)

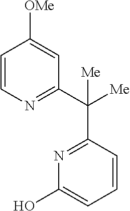 [SMILES: COc1ccnc(C(C)(C)c2cccc(O)n2)c1]

Yellow solid, ¹H NMR (600 MHz, Chloroform-d) δ 8.47 (d, J=5.8 Hz, 1H), 7.32 (dd, J=9.2, 7.0 Hz, 1H), 6.85 (d, J=2.4 Hz, 1H), 6.72 (dd, J=5.7, 2.4 Hz, 1H), 6.37 (dd, J=9.1, 0.9 Hz, 1H), 6.18 (dd, J=6.9, 1.0 Hz, 1H), 3.84 (s, 3H), 1.71 (s, 6H); ¹³C NMR (151 MHz, CDCl₃) δ 166.60, 164.76, 163.61, 153.41, 150.57, 140.83, 118.51, 107*.48, 107.19, 101.29, 55.24, 42.95, 27.59. HRMS (ESI-TOF) m/z Calcd for C₁₄H₁₇N₂O₂⁺ [M+H]⁺ 245.1290, found 245.1291.

### Example 36: 6-(2-(5-chloropyridin-2-yl)-1-phenylpropan-2-yl)pyridin-2-ol (L39)

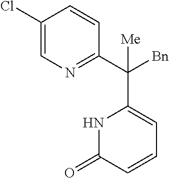 [SMILES: CC(Cc1ccccc1)(c1ccc(Cl)cn1)c1cccc(=O)[nH]1]

Yellow solid, ¹H NMR (600 MHz, Chloroform-d) δ 8.72 (dd, J=2.6, 0.8 Hz, 1H), 7.64 (dd, J=8.5, 2.6 Hz, 1H), 7.25 (d, J=7.6 Hz, 1H), 7.22-7.20 (m, 1H), 7.19-7.12 (m, 3H), 6.81-6.73 (m, 2H), 6.42 (dd, J=9.2, 0.9 Hz, 1H), 5.98 (dd, J=7.0, 1.0 Hz, 1H), 3.41 (q, J=13.4 Hz, 2H), 1.59 (s, 3H); ¹³C NMR (151 MHz, CDCl₃) δ 163.57, 160.39, 158.10, 150.90, 147.89, 140.58, 136.98, 136.31, 130.18, 127.98, 126.85, 122.09, 118.97, 103.15, 48.12, 46.85, 21.39. HRMS (ESI-TOF) m/z Calcd for C₁₉H₁₈ClN₂O⁺ [M+H]⁺ 325.1108, found 325.1112.

### Process for Making Compounds of Formula (2)

### Example 37: Ligand Effect on Dehydrogenation of Free Carboxylic Acid

The purpose of this example is to demonstrate the use of ligands (L-1) in an illustrative embodiment of the process for making a compound of formula (2):

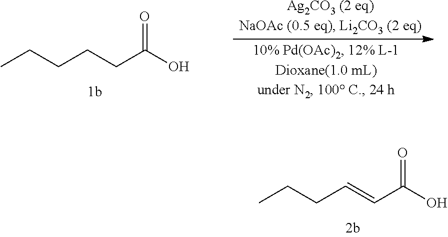

Reaction conditions were as follows: 1b (0.1 mmol), Pd(OAc)₂ (10 mol %), Ligand (12 mol %), Ag₂CO₃ (2.0 equiv), Li₂CO₃ (2.0 equiv), NaOAc (0.5 equiv), 1,4-dioxane (1.0 mL), 100° C., N₂, 24 h. The yields were determined by ¹H NMR using dibromomethane as internal standard (Table 1).

TABLE 1
Effect of Ligand L-1 on Yield of 2b

| Ligand | Structure | Yield (%) |
|---|---|---|
| L1 | 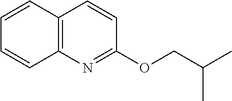 | 0 |
| L2 | 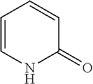 | 0 |
| L3 | 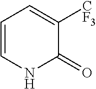 [SMILES: O=c1[nH]cccc1C(F)(F)F] | 0 |
| L4 | 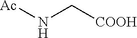 | 0 |
| L5 | 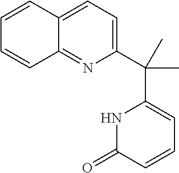 [SMILES: CC(C)(c1ccc2ccccc2n1)c1cccc(=O)[nH]1] | 15 |
| L6 | 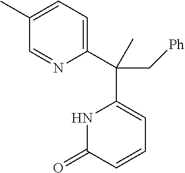 [SMILES: Cc1ccc(C(C)(Cc2ccccc2)c2cccc(=O)[nH]2)nc1] | 6 |
| L7 | 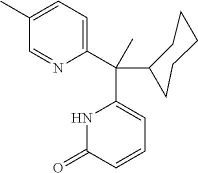 [SMILES: Cc1ccc(C(C)(c2cccc(=O)[nH]2)C2CCCCC2)nc1] | 4 |
| L8 | 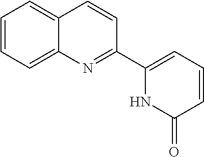 [SMILES: O=c1cccc(-c2ccc3ccccc3n2)[nH]1] | 71 |
| L9 | 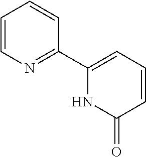 [SMILES: O=c1cccc(-c2ccccn2)[nH]1] | 35 |
| L10 | 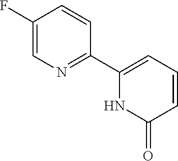 [SMILES: O=c1cccc(-c2ccc(F)cn2)[nH]1] | 32 |
| L11 | 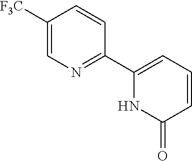 [SMILES: O=c1cccc(-c2ccc(C(F)(F)F)cn2)[nH]1] | 41 |
| L12 | 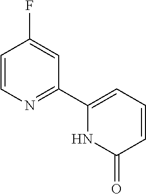 [SMILES: O=c1cccc(-c2cc(F)ccn2)[nH]1] | 28 |
| L13 | 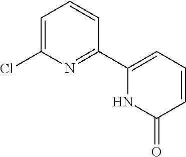 [SMILES: O=c1cccc(-c2cccc(Cl)n2)[nH]1] | 64 |
| L14 | 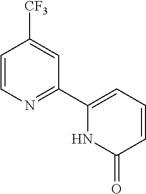 [SMILES: O=c1cccc(-c2cc(C(F)(F)F)ccn2)[nH]1] | 65 |
| L15 | 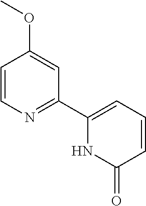 | 32 |
| L16 | 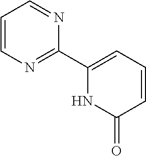 [SMILES: O=c1cccc(-c2ncccn2)[nH]1] | 30 |
| L17 | 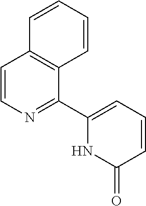 [SMILES: O=c1cccc(-c2nccc3ccccc23)[nH]1] | 67 |
| L18 | 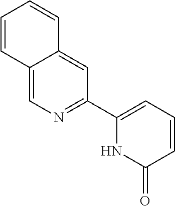 [SMILES: O=c1cccc(-c2cc3ccccc3cn2)[nH]1] | 22 |
| L19 | 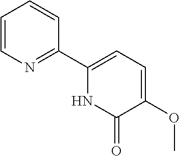 | 23 |

### Example 38: Ligand Loading Effects

The purpose of this example is to demonstrate the effect of ligand (L-1) loading in an illustrative embodiment of the process for making a compound of formula (2):

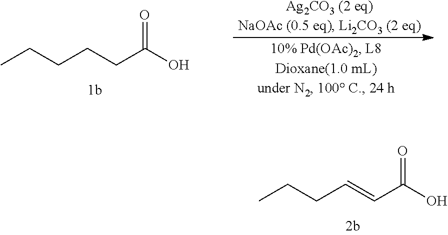

Reaction conditions were as follows: 1b (0.1 mmol), Pd(OAc)₂ (10 mol %), L8, Ag₂CO₃ (2.0 equiv), Li₂CO₃ (2.0 equiv), NaOAc (0.5 equiv), 1,4-dioxane (1.0 mL), 100° C., N₂, 24 h. The yields were determined by ¹H NMR using dibromomethane as internal standard (Table 2)

TABLE 2
Effect of Ligand (L-1) loading on Yield of 2b

| Entry | Ligand amount (mol %) | Yield (%) |
|---|---|---|
| 1 | 10 | 68 |
| 2 | 11 | 72 |
| 3 | 13 | 60 |
| 4 | 16 | 58 |
| 5 | 19 | 38 |
| 6 | 22 | 36 |
| 7 | none | 0 |

### Example 39: Palladium Loading Effects

The purpose of this example is to demonstrate the effect of palladium (II) source loading in an illustrative embodiment of the process for making a compound of formula (2):

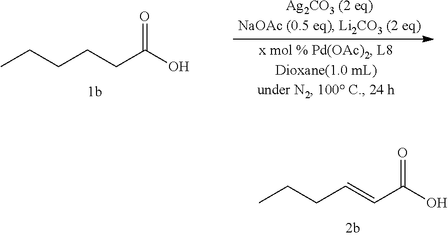

Reaction conditions were as follows: 1b (0.1 mmol), Pd(OAc)₂ (10 mol %), L8 (1.1 equiv. relative to Pd), Ag₂CO₃ (2.0 equiv), Li₂CO₃ (2.0 equiv), NaOAc (0.5 equiv), 1,4-dioxane (1.0 mL), 100° C., N₂, 24 h. The yields determined by ¹H NMR using dibromomethane as internal standard (Table 3).

TABLE 3
Palladium Loading Effects

| Entry | Pd amount (mol %) | Yield (%) |
|---|---|---|
| 1 | 10 | 72 |
| 2 | 3 | 60 |
| 3 | 2 | 47 |
| 4 | 1 | 44 |

### Example 40: Solvent Effects

The purpose of this example is to demonstrate the effect of solvent in an illustrative embodiment of the process for making a compound of formula (2):

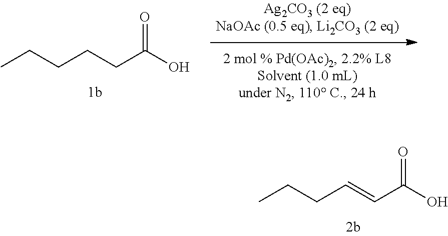

Reaction conditions were as follows: 1b (0.1 mmol), Pd(OAc)₂ (2 mol %), L8 (2.2 mol %), Ag₂CO₃ (2.0 equiv), Li₂CO₃ (2.0 equiv), NaOAc (0.5 equiv), solvent, 110° C., N₂, 24 h. The yields determined by ¹H NMR using dibromomethane as internal standard (Table 4).

TABLE 4
Solvent Effects

| Entry | Solvent | Yield (%) |
|---|---|---|
| 1 | 1.0 mL Dioxane | 49 |
| 2 | 0.2 mL Dioxane + 0.8 mL t-Amyl-OH | 70 |
| 3 | 0.2 mL Dioxane + 0.2 mL t-Amyl-OH | 47 |
| 4 | 0.5 mL Dioxane + 0.5 mL t-Amyl-OH | 61 |
| 5 | 0.2 mL Dioxane + 0.8 mL t-Bu—OH | 53 |
| 6 | 0.2 mL Dioxane + 0.2 mL t-Bu—OH | 49 |
| 7 | 0.5 mL Dioxane + 0.5 mL t-Bu—OH | 50 |

### Example 41: Base Amount Effects

The purpose of this example is to demonstrate the effect of amount of base in an illustrative embodiment of the process for making a compound of formula (2):

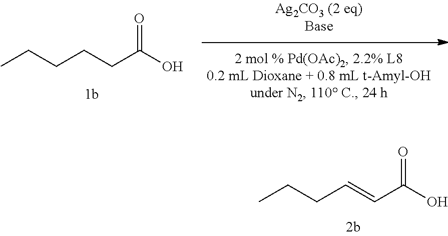

Reaction conditions were as follows: 1b (0.1 mmol), Pd(OAc)₂ (2 mol %), L8 (2.2 mol %), Ag₂CO₃ (2.0 equiv), Base, 1,4-dioxane (0.2 mL), t-Amyl-OH (0.8 mL), 110° C., N₂, 24 h. The yields determined by H NMR using dibromomethane as internal standard (Table 5).

TABLE 5
Effect of Base on Yield of 2b.

| Entry | Base | Yield (%) |
|---|---|---|
| 1 | 1.0 eq Li₂CO₃ + 0.25 eq NaOAc | 46 |
| 2 | 1.0 eq Li₂CO₃ + 0.50 eq NaOAc | 20 |
| 3 | 1.0 eq Li₂CO₃ + 1.00 eq NaOAc | 65 |
| 4 | 2.0 eq Li₂CO₃ + 0.25 eq NaOAc | 25 |
| 5 | 2.0 eq Li₂CO₃ + 0.50 eq NaOAc | 70 |
| 6 | 2.0 eq Li₂CO₃ + 1.00 eq NaOAc | 55 |
| 7 | 4.0 eq Li₂CO₃ + 0.25 eq NaOAc | 38 |
| 8 | 4.0 eq Li₂CO₃ + 0.50 eq NaOAc | 58 |
| 9 | 4.0 eq Li₂CO₃ + 1.00 eq NaOAc | 44 |

### Example 42A: Effects of Oxidant

The purpose of this example is to demonstrate the effect of oxidant in an illustrative embodiment of the process for making a compound of formula (2):

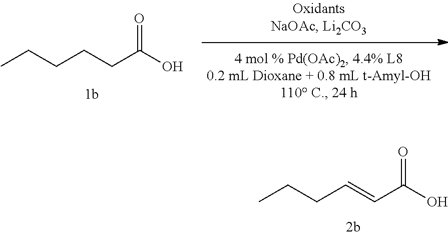

Reaction conditions were as follows: 1b (0.1 mmol), Pd(OAc)₂ (4 mol %), L8 (4.4 mol %), oxidants, Li₂CO₃ (2.0 equiv), NaOAc (0.5 equiv), 1,4-dioxane (0.2 mL), t-Amyl-OH (0.8 mL), 110° C., 24 h. The yields were determined by ¹H NMR using dibromomethane as internal standard. (Table 6A).

TABLE 6A
Effect of Oxidant on Yield of 2b.

| Entry | Oxidant(s) | Yield (%) |
|---|---|---|
| 1 | TBHP in water, 2 eq.^{‡} | 60 |
| 2 | CMHP in water, 2 eq. | 56 |
| 3 | AcOOtBu, 2 eq. | 30 |
| 4 | BzOOtBu, 2 eq. | 26 |
| 5 | BzOOBz, 2 eq. | 0 |
| 6 | lauroyl peroxide, 2 eq. | <5 |
| 7 | tBuOOtBu, 2 eq. | 0 |
| 8 | O₂ (1 atm), BQ (1 eq.)^{§} | 25 |
| 9 | O₂ (1 atm), 2,5-di-tBu—BQ (1 eq.)^{§} | 37 |
| 10 | O₂ (1 atm), BQ (1 eq.), CuBr (0.1 eq.)^{§} | 61 |
| 11 | O₂ (3 atm), BQ (1 eq.), CuBr (0.1 eq.)** | 71 |
| 12 | O₂ (1 atm), BQ (1 eq.), CuBr (0.5 eq.)^{§} | 45 |
| 13 | O₂ (1 atm), BQ (1 eq.), CuCl (0.1 eq.)^{§} | 59 |
| 14 | O₂ (1 atm), BQ (1 eq.), CuCl₂ (0.1 eq.)^{§} | 52 |
| 15 | O₂ (1 atm), BQ (1 eq.), CuI (0.1 eq.)^{§} | 21 |

^{‡}at 80° C.
^{§}1 atm O₂ is used. BQ (1,4-benzoquinone, 1.0 equiv.), CuBr (0.1 equiv.) and DMF (dimethylformamide, 0.8 mL) are added, 8 hours.
**In a pressurized vessel, 1.0 mmol scale, 3 atm O₂ is used. BQ (1,4-benzoquinone, 1.0 equiv.), CuBr (0.1 equiv.) and DMF (dimethylformamide, 8.0 mL) are added, 8 hours.

### Example 42B: Effects Temperature and Reaction Time

The purpose of this example is to demonstrate the scope of compounds of formula (2) that were made from compounds of formula (1), respectively, in an exemplary reaction comparing effects of temperature and reaction times

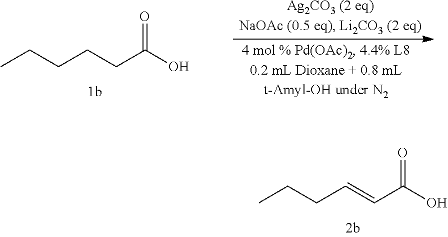

Reaction conditions were as follows: 1b (0.1 mmol), Pd(OAc)₂ (4 mol %), L8 (4.4 mol %), Ag₂CO₃ (2.0 equiv), Li₂CO₃ (2.0 equiv), NaOAc (0.5 equiv), 1,4-dioxane (0.2 mL), t-Amyl-OH (0.8 mL), under N₂. The yields were determined by 1H NMR using dibromomethane as internal standard (Table 6B).

TABLE 6B
Effect of Temperature and Reaction Time on Yield of 2b.

| Entry | Conditions | Yield (%) |
|---|---|---|
| 1 | 80 C., 14 h | 71 |
| 2 | 80 C., 20 h | 79 |
| 3 | 80 C., 26 h | 77 |
| 4 | 100 C., 8 h | 76 |
| 5 | 100 C., 16 h | 82 |
| 6 | 100 C., 24 h | 72 |
| 7 | 110 C., 2 h | 67 |
| 8 | 110 C., 4 h | 79 |
| 9 | 110 C., 7 h | 78 |

### Example 43: Substrate Scope for Dehydrogenation Reaction

The purpose of this example is to demonstrate the scope of compounds of formula (2) that were made from compounds of formula (1), respectively, in an exemplary reaction comparing the use of Ag₂CO₃ and TBHP as oxidants:

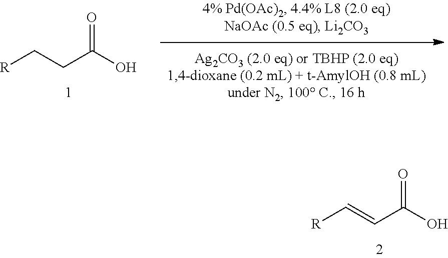

General Procedure for β-C(sp³)-H Dehydrogenation: In a sealed tube equipped with a magnetic stir bar was charged with the appropriate carboxylic acid substrate (0.10 mmol), Ag₂CO₃ (55.0 mg, 0.2 mmol), Li₂CO₃ (14.8 mg, 0.2 mmol) and NaOAc (4.1 mg, 0.05 mmol). A solution of Pd(OAc)₂ (0.9 mg, 4 mol %) and L8 (1.0 mg, 4.4 mol %) in 1,4-dioxane (0.2 mL) was premixed added to the tube. t-Amyl-OH (0.8 ml) was then added before the tube was briefly flushed with nitrogen. Subsequently the vial was capped and closed tightly. The reaction mixture was then stirred at the rate of 300 rpm at 110° C. for 24 h. After being allowed to cool to room temperature, the mixture was acidified with 20 μL of formic acid and sonicated for 30 seconds. The mixture was passed through a pad of Celite with a solvent mixture (methanol:formicacid:DCM=5:5:90) as the eluent to remove any insoluble precipitate. The resulting solutions was concentrated, and the residual mixture was purified using reverse phase chromatography (H₂O:acetonitrile=10:1 to 1:4). Results are summarized in the table below.

TABLE
Synthesis of Compounds of Formula (2).

| | | Yield (%) | | |
|---|---|---|---|---|
| Product | Structure | Ag₂CO₃ | THBP | O₂ |
| 2a | 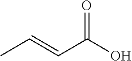 | 78 | 54 | 62^{†} |
| 2b | 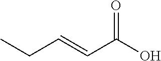 | 81 | 60 | 71^{‡} |
| 2c | 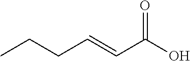 | 80 | 58 | 58^{†} |
| 2d | 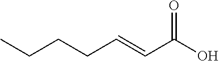 | 76 | 52 | 56^{†} |
| 2e | 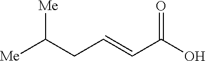 | 82 | — | — |
| 2f | 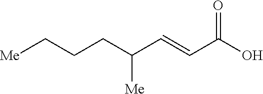 [SMILES: CCCCC(C)/C=C/C(=O)O] | 77 | — | — |
| 2g | 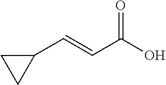 | 81 | — | — |
| 2h | 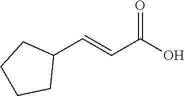 | 72 | — | — |
| 2i | 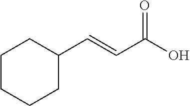 [SMILES: O=C(O)/C=C/C1CCCCC1] | 77 | — | — |
| 2j | 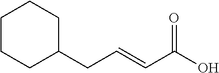 [SMILES: O=C(O)/C=C/CC1CCCCC1] | 87 | — | — |
| 2k | 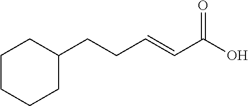 [SMILES: O=C(O)/C=C/CCC1CCCCC1] | 74^{§} | — | — |
| 2l | 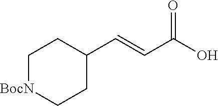 [SMILES: CC(C)(C)OC(=O)N1CCC(/C=C/C(=O)O)CC1] | 79 | — | — |
| 2m | 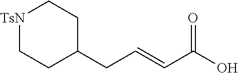 [SMILES: Cc1ccc(S(=O)(=O)N2CCC(C/C=C/C(=O)O)CC2)cc1] | 71 | — | — |
| 2n | 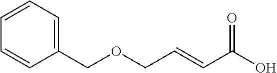 [SMILES: O=C(O)/C=C/COCc1ccccc1] | 77 | — | — |
| 2o | 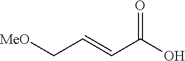 | 67 | — | — |
| 2p | 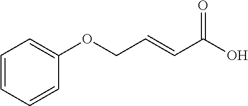 [SMILES: O=C(O)/C=C/COc1ccccc1] | 68 | — | — |
| 2q | 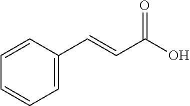 [SMILES: O=C(O)/C=C/c1ccccc1] | 82 | 57* | 67^{†} |
| 2r | 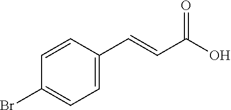 [SMILES: O=C(O)/C=C/c1ccc(Br)cc1] | 85 | 61* | 59^{†} |
| 2s | 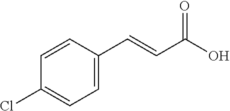 [SMILES: O=C(O)/C=C/c1ccc(Cl)cc1] | 80 | 53* | 52^{†} |
| 2t | 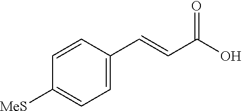 [SMILES: CSc1ccc(/C=C/C(=O)O)cc1] | 65^{§} | — | — |
| 2u | 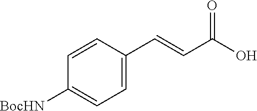 [SMILES: CC(C)(C)OC(=O)Nc1ccc(/C=C/C(=O)O)cc1] | 42 | — | — |
| 2v | 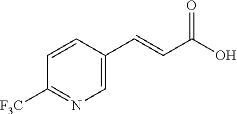 [SMILES: O=C(O)/C=C/c1ccc(C(F)(F)F)nc1] | 76^{§} | — | — |
| 2w | 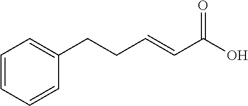 [SMILES: O=C(O)/C=C/CCc1ccccc1] | 71^{§} | — | — |
| 2x | 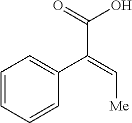 [SMILES: C/C=C(/C(=O)O)c1ccccc1] | 65 | — | — |
| 2y | 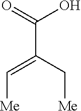 | 51 | — | — |
| 2z | 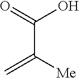 | 42^{§} | — | — |
| 2aa | 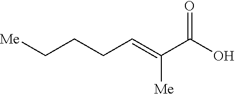 | 54^{§} | — | — |
| 2ab | 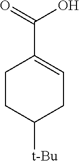 [SMILES: CC(C)(C)C1CC=C(C(=O)O)CC1] | 56^{§} | — | — |
| 2ac | 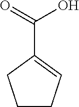 | 43^{§} | — | — |
| 2ad | 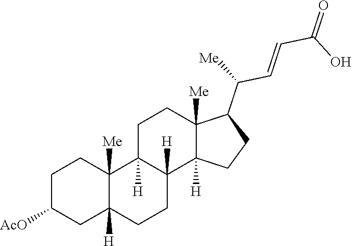 | 56^{§} | 39 | — |
| 2ae | 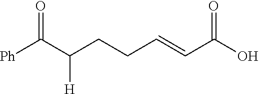 [SMILES: O=C(O)/C=C/CCCC(=O)c1ccccc1] | 59^{§} | — | — |
| 2af | 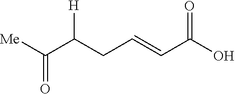 | 54 | — | — |
| 2ag | 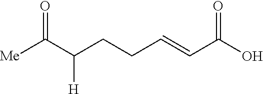 [SMILES: CC(=O)CCC/C=C/C(=O)O] | 62 | 40* | — |
| 2ah | 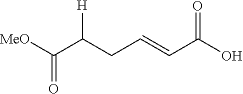 [SMILES: COC(=O)CC/C=C/C(=O)O] | 75 | 59* | — |
| 2ai | 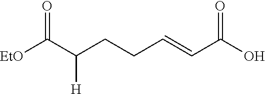 [SMILES: CCOC(=O)CCC/C=C/C(=O)O] | 67 | 47* | 53^{†} |
| 2aj | 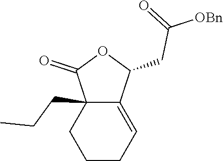 [SMILES: CCC[C@]12CCCC=C1[C@@H](CC(=O)OCc1ccccc1)OC2=O] | 41** | — | — |

*2 equiv. of TBHP in water was used as oxidant instead of Ag₂CO₃; 80° C.
^{†}1 atm O₂ is used instead of Ag₂CO₃; BQ (1,4-benzoquinone, 1.0 equiv.), CuBr (0.1 equiv.) and DMF (dimethylformamide, 0.8 mL) are added; 8 h.
^{‡}In a pressurized vessel, 1.0 mmol scale, 3 atm O₂ is used instead of Ag₂CO₃; BQ (1,4-benzoquinone, 1.0 equiv.), CuBr (0.1 equiv.) and DMF (dimethylformamide, 8.0 mL) are added; 8 h.
^{§}at 110° C.
**Benzyl acrylate (2.0 equiv.) is added; HFIP (hexafluoro-2-propanol, 0.8 mL) is used as co-solvent instead of tert-amyl alcohol.

### Example 44: (E)-but-2-enoic acid (2a)

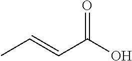

Following General Procedure on 0.1 mmol scale. Due to the volatility of the product, the yield was determined by ¹H NMR analysis of the crude product using CH₂Br₂ (0.1 mmol, 7 μL) as the internal standard (78% yield). ¹H NMR (600 MHz, Chloroform-d) δ 7.10 (dd, J=15.5, 6.9 Hz, 1H), 5.86 (dd, J=15.6, 1.8 Hz, 1H), 1.92 (dd, J=7.0, 1.8 Hz, 3H). ¹³C NMR (151 MHz, CDCl3) δ 172.19, 147.55, 122.23, 18.12. The NMR data matches the reported data (31).

### Example 45: (E)-hex-2-enoic acid (2b)

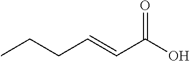

Following General Procedure on 0.1 mmol scale. Purification by reverse phase column (water:acetonitrile=10:1 to 1:4) afforded the title compound (colorless oil, 9.3 mg, 81% yield). ¹H NMR (500 MHz, Chloroform-d) 67.08 (dt, J=15.6, 7.0 Hz, 1H), 5.83 (dt, J=15.6, 1.6 Hz, 1H), 2.22 (qd, J=7.2, 1.6 Hz, 2H), 1.51 (h, J=7.4 Hz, 2H), 0.95 (t, J=7.4 Hz, 3H). ¹³C NMR (126 MHz, CDCl₃) δ 171.65, 152.37, 120.77, 34.45, 21.30, 13.79. HRMS (ESI-TOF) Calcd for C₆H₁₁O₂[M+H]⁺: 115.0754; found: 115.0755.

### Example 46: (E)-hept-2-enoic acid (2c)

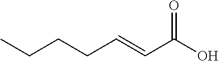

Following General Procedure on 0.1 mmol scale. Purification by reverse phase column (water:acetonitrile=10:1 to 1:4) afforded the title compound (colorless oil, 10.3 mg, 80% yield). ¹H NMR (500 MHz, Chloroform-d) δ 7.08 (dt, J=15.6, 7.0 Hz, 1H), 5.83 (d, J=15.6 Hz, 1H), 2.24 (qd, J=7.1, 1.6 Hz, 2H), 1.50-1.41 (m, 2H), 1.39-1.34 (m, 2H), 0.92 (t, J=7.3 Hz, 3H). ¹³C NMR (151 MHz, Chloroform-d) δ 171.22, 152.45, 120.40, 32.02, 29.97, 22.22, 13.81. HRMS (ESI-TOF) Calcd for C₇H₁₃O₂[M+H]⁺: 129.0910; found: 129.0909.

### Example 47: (E)-oct-2-enoic acid (2d)

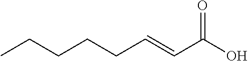

Following General Procedure on 0.1 mmol scale. Purification by reverse phase column (water:acetonitrile=10:1 to 1:4) afforded the title compound (white solid, 10.8 mg, 76% yield). ¹H NMR (400 MHz, Chloroform-d) δ 7.09 (dt, J=15.6, 7.0 Hz, 1H), 5.83 (dt, J=15.6, 1.7 Hz, 1H), 2.23 (qd, J=7.2, 1.6 Hz, 2H), 1.47 (td, J=11.5, 9.4, 4.9 Hz, 2H), 1.33-1.28 (m, 4H), 0.93-0.86 (m, 3H). ¹³C NMR (151 MHz, CDCl₃) δ 171.72, 152.51, 120.52, 32.29, 31.31, 27.56, 22.42, 13.96. HRMS (ESI-TOF) Calcd for C₈H₁₅O₂[M+H]⁺: 143.1067; found: 143.1062.

### Example 48: (E)-5-methylhex-2-enoic acid (2e)

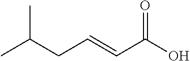

Following General Procedure on 0.1 mmol scale. Purification by reverse phase column (water:acetonitrile=10:1 to 1:4) afforded the title compound (colorless oil, 10.5 mg, 82% yield). ¹H NMR (500 MHz, Chloroform-d) δ 7.06 (dt, J=15.3, 7.5 Hz, 1H), 5.82 (d, J=15.5 Hz, 1H), 2.12 (t, J=7.2 Hz, 2H), 1.78 (dt, J=13.4, 6.7 Hz, 1H), 0.93 (d, J=6.7 Hz, 6H). ¹³C NMR (151 MHz, Chloroform-d) δ 172.28, 151.45, 121.82, 41.66, 27.90, 22.48. HRMS (ESI-TOF) Calcd for C₇H₁₃O₂[M+H]⁺: 129.0910; found: 129.0905.

### Example 49: (E)-4-methyloct-2-enoic acid (2f)

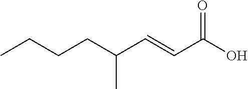

Following General Procedure on 0.1 mmol scale. Purification by reverse phase column (water:acetonitrile=10:1 to 1:4) afforded the title compound (white solid, 12.0 mg, 77% yield). ¹H NMR (600 MHz, Chloroform-d) δ 9.50 (br s, 1H), 6.95 (dd, J=15.6, 7.9 Hz, 1H), 5.78 (d, J=15.6 Hz, 1H), 2.31 (hept, J=6.9 Hz, 1H), 1.43-1.19 (m, 6H), 1.04 (d, J=6.8 Hz, 3H), 0.88 (t, J=7.1 Hz, 3H). ¹³C NMR (151 MHz, Chloroform-d) δ 172.57, 157.22, 119.59, 36.74, 35.80, 29.51, 22.85, 19.43, 14.13. HRMS (ESI-TOF) Calcd for C₉H₁₅O₂[M−H]⁻: 155.1072; found: 155.1078.

### Example 50: (E)-3-cyclopropylacrylic acid (2g)

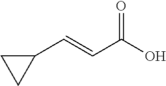

Following General Procedure on 0.1 mmol scale. Purification by reverse phase column (water:acetonitrile=10:1 to 1:4) afforded the title compound (colorless oil, 9.1 mg, 81% yield). ¹H NMR (600 MHz, Chloroform-d) δ 6.55 (dd, J=15.4, 10.2 Hz, 1H), 5.92 (d, J=15.4 Hz, 1H), 1.63 (dtt, J=12.5, 8.3, 4.2 Hz, 1H), 1.04-0.98 (m, 3H), 0.74-0.67 (m, 3H). ¹³C NMR (151 MHz, Chloroform-d) δ 172.02, 157.29, 117.52, 14.79, 9.16. HRMS (ESI-TOF) Calcd for C₆H₉O₂ [M+H]⁺: 113.0597; found: 113.0598.

### Example 51: (E)-3-cyclopentylacrylic acid (2h)

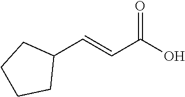

Following General Procedure on 0.1 mmol scale. Purification by reverse phase column (water:acetonitrile=10:1 to 1:4) afforded the title compound (white solid, 10.1 mg, 72% yield). ¹H NMR (500 MHz, Chloroform-d) δ 11.62 (br s, 1H), 7.05 (dd, J=15.5, 8.0 Hz, 1H), 5.80 (d, J=15.5 Hz, 1H), 2.62 (h, J=8.0 Hz, 1H), 1.85 (dq, J=12.3, 6.7 Hz, 2H), 1.70 (qd, J=10.7, 9.7, 5.9 Hz, 2H), 1.62 (qd, J=8.9, 7.3, 5.3 Hz, 2H), 1.41 (dq, J=12.5, 7.8 Hz, 2H). ¹³C NMR (126 MHz, Chloroform-d) δ 172.71, 156.57, 118.97, 43.04, 32.46, 25.40. HRMS (ESI-TOF) Calcd for C₈H₁₃O₂[M+H]⁺: 141.0910; found: 141.0907.

### Example 52: (E)-3-cyclohexylacrylic acid (2i)

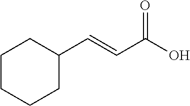 [SMILES: O=C(O)/C=C/C1CCCCC1]

Following General Procedure on 0.1 mmol scale. Purification by reverse phase column (water:acetonitrile=10:1 to 1:4) afforded the title compound (white solid, 11.8 mg, 77% yield). ¹H NMR (600 MHz, Chloroform-d) δ 9.93 (br s, 1H), 7.01 (dd, J=15.9, 6.8 Hz, 1H), 5.77 (d, J=15.8 Hz, 1H), 2.16 (q, J=9.7, 9.3 Hz, 1H), 1.83-1.72 (m, 4H), 1.72-1.64 (m, 1H), 1.36-1.23 (m, 2H), 1.24-1.10 (m, 3H). ¹³C NMR (151 MHz, Chloroform-d) δ 172.79, 157.07, 118.65, 40.64, 31.68, 26.03, 25.80. HRMS (ESI-TOF) Calcd for C₉H₁₄O₂ [M+H]⁺: 155.1067; found: 155.1065.

### Example 53: (E)-4-cyclohexylbut-2-enoic acid (2j)

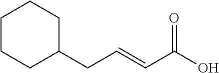 [SMILES: O=C(O)/C=C/CC1CCCCC1]

Following General Procedure on 0.1 mmol scale. Purification by reverse phase column (water:acetonitrile=10:1 to 1:4) afforded the title compound (white solid, 14.6 mg, 87% yield). 1H NMR (600 MHz, Chloroform-d) δ 7.07 (dt, J=15.3, 7.5 Hz, 1H), 5.81 (d, J=15.6 Hz, 1H), 2.13 (td, J=7.1, 1.5 Hz, 2H), 1.72-1.63 (m, 5H), 1.45 (dddd, J=14.8, 11.3, 5.8, 3.4 Hz, 1H), 1.25-1.11 (m, 3H), 0.98-0.89 (m, 2H). 13C NMR (151 MHz, CDCl3) δ 172.20, 151.36, 121.58, 40.24, 37.21, 33.12, 26.31, 26.18. HRMS (ESI-TOF) Calcd for C10H17O2 [M+H]+: 169.1223; found: 169.1218.

### Example 54: (E)-5-cyclohexylpent-2-enoic acid (2k)

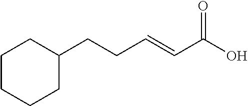 [SMILES: O=C(O)/C=C/CCC1CCCCC1]

Following General Procedure on 0.1 mmol scale. Purification by reverse phase column (water:acetonitrile=10:1 to 1:4) afforded the title compound (white solid, 13.4 mg, 74% yield). ¹H NMR (600 MHz, Chloroform-d) δ 7.09 (dt, J=15.5, 6.9 Hz, 1H), 5.82 (d, J=15.6 Hz, 1H), 2.24 (q, J=7.4, 6.8 Hz, 2H), 1.74-1.60 (m, 5H), 1.35 (q, J=7.3 Hz, 2H), 1.28-1.07 (m, 4H), 0.89 (qd, J=14.0, 12.9, 3.7 Hz, 2H). ¹³C NMR (151 MHz, Chloroform-d) δ 172.05, 152.87, 120.40, 37.09, 35.46, 33.14, 29.74, 26.59, 26.26. HRMS (ESI-TOF) Calcd for C₁₁H₁₇O₂[M−H]⁻: 181.1229; found: 181.1236.

### Example 55: (E)-3-(1-(tert-butoxycarbonyl)piperidin-4-yl)acrylic acid (21)

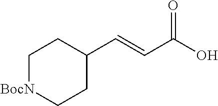 [SMILES: CC(C)(C)OC(=O)N1CCC(/C=C/C(=O)O)CC1]

Following General Procedure on 0.1 mmol scale. Purification by reverse phase column (water:acetonitrile=10:1 to 1:4) afforded the title compound (white solid, 21.8 mg, 79% yield). ¹H NMR (500 MHz, Chloroform-d) δ 9.17 (br s, 1H), 6.85 (d, J=10.4 Hz, 1H), 5.78 (d, J=14.3 Hz, 1H), 4.27-3.92 (m, 2H), 2.90-2.51 (m, 2H), 2.37-2.17 (m, 1H), 1.76-1.63 (m, 2H), 1.44 (s, 9H), 1.34-1.28 (m, 2H). ¹³C NMR (126 MHz, Chloroform-d) δ 172.23, 154.91, 152.38, 121.38, 79.78, 43.71, 38.68, 30.76, 28.58. HRMS (ESI-TOF) Calcd for C₁₃H₂₁NO₄Na [M+Na]⁺: 278.1363; found: 278.1364.

### Example 56: benzyl (E)-4-(1-tosylpiperidin-4-yl)but-2-enoate (2m)

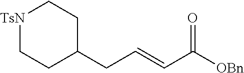 [SMILES: Cc1ccc(S(=O)(=O)N2CCC(C/C=C/C(=O)OCc3ccccc3)CC2)cc1]

Following General Procedure on 0.1 mmol scale. The product was protected using benzyl alcohol for the ease of isolation. Purification by preparative TLC (hexane:ethyl acetate=4:1) afforded the title compound (white solid, 29.3 mg, 71% yield). ¹H NMR (500 MHz, Chloroform-d) δ 7.65-7.58 (m, 2H), 7.36 (d, J=3.9 Hz, 4H), 7.34-7.30 (m, 3H), 6.88 (dt, J=15.4, 7.5 Hz, 1H), 5.85 (dt, J=15.6, 1.4 Hz, 1H), 5.16 (s, 2H), 3.76 (dq, J=13.0, 2.9, 2.4 Hz, 2H), 2.43 (s, 3H), 2.21 (td, J=11.4, 2.6 Hz, 2H), 2.12 (td, J=6.0, 3.0 Hz, 2H), 1.78-1.68 (m, 2H), 1.39-1.28 (m, 3H). ¹³C NMR (126 MHz, Chloroform-d) δ 166.08, 146.91, 143.46, 135.99, 133.14, 129.61, 128.57, 128.25, 127.72, 122.82, 66.17, 46.26, 38.66, 34.71, 31.28, 21.53. HRMS (ESI-TOF) Calcd for C₂₃H₂₈NO₄S [M+H]⁺: 414.1734; found: 414.1734.

### Example 57: (E)-4-(benzyloxy)but-2-enoic acid (2n)

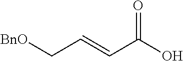 [SMILES: O=C(O)/C=C/COCc1ccccc1]

Following General Procedure on 0.1 mmol scale. Purification by reverse phase column (water:acetonitrile=10:1 to 1:4) afforded the title compound (white solid, 14.7 mg, 77% yield). ¹H NMR (500 MHz, Chloroform-d) δ 11.17 (br s, 1H), 7.40-7.28 (m, 5H), 7.10 (d, J=15.6 Hz, 1H), 6.17 (d, J=15.6 Hz, 1H), 4.59 (s, 2H), 4.22 (s, 2H). ¹³C NMR (126 MHz, Chloroform-d) δ 171.79, 147.17, 137.70, 128.64, 128.03, 127.79, 120.75, 73.00, 68.61. HRMS (ESI-TOF) Calcd for C₁₁H₁₂O₃Na [M+Na]⁺: 215.0678; found: 215.0673.

### Example 58: (E)-4-methoxybut-2-enoic acid (20)

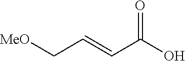

Following General Procedure on 0.1 mmol scale. Purification by reverse phase column (water:acetonitrile=10:1 to 1:4) afforded the title compound (colorless oil, 7.8 mg, 67% yield). ¹H NMR (500 MHz, Chloroform-d) δ 7.06 (dt, J=15.8, 4.1 Hz, 1H), 6.08 (dt, J=15.9, 2.2 Hz, 1H), 4.12 (dd, J=4.3, 2.1 Hz, 2H), 3.41 (s, 3H). ¹³C NMR (126 MHz, Chloroform-d) δ 171.29, 147.11, 120.51, 71.13, 58.91. HRMS (ESI-TOF) Calcd for C₅H₇O₃ [M−H]⁻: 115.0400; found: 115.0398.

### Example 59: (E)-4-phenoxybut-2-enoic acid (2p)

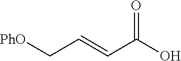 [SMILES: O=C(O)/C=C/COc1ccccc1]

Following General Procedure on 0.1 mmol scale. Purification by reverse phase column (water:acetonitrile=10:1 to 1:4) afforded the title compound (white solid, 12.1 mg, 68% yield). ¹H NMR (600 MHz, Chloroform-d) δ 7.30 (t, J=8.0 Hz, 2H), 7.20 (dt, J=15.7, 3.9 Hz, 1H), 6.99 (t, J=7.4 Hz, 1H), 6.92 (d, J=8.1 Hz, 2H), 6.24 (dd, J=15.7, 2.4 Hz, 1H), 4.74 (dd, J=3.9, 2.1 Hz, 2H). ¹³C NMR (126 MHz, Chloroform-d) δ 171.21, 158.07, 145.47, 129.76, 121.61, 121.22, 114.78, 66.40. HRMS (ESI-TOF) Calcd for C₁₀H₉O₃ [M−H]⁻: 177.0557; found: 177.0558.

### Example 60: cinnamic acid (2q)

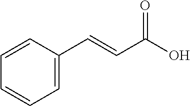 [SMILES: O=C(O)/C=C/c1ccccc1]

Following General Procedure on 0.1 mmol scale. Purification by reverse phase column (water:acetonitrile=10:1 to 1:4) afforded the title compound (white solid, 12.1 mg, 82% yield). ¹H NMR (500 MHz, Chloroform-d) δ 12.39 (br s, 1H), 7.82 (d, J=16.0 Hz, 1H), 7.59-7.53 (m, 2H), 7.44-7.40 (m, 3H), 6.47 (d, J=16.0 Hz, 1H). ¹³C NMR (126 MHz, Chloroform-d) δ 172.83, 147.28, 134.17, 130.90, 129.11, 128.53, 117.48. HRMS (ESI-TOF) Calcd for C₉H₉O₂ [M+H]⁺: 149.0597; found: 149.0596.

### Example 61: (E)-3-(4-bromophenyl)acrylic acid (2r)

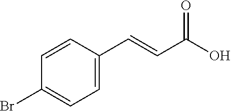 [SMILES: O=C(O)/C=C/c1ccc(Br)cc1]

Following General Procedure on 0.1 mmol scale. Purification by reverse phase column (water:acetonitrile=10:1 to 1:4) afforded the title compound (white solid, 19.1 mg, 85% yield). ¹H NMR (400 MHz, Acetone-d₆) δ 7.71-7.60 (m, 5H), 6.58 (d, J=16.1 Hz, 1H). ¹³C NMR (126 MHz, Acetone-d₆) δ 166.64, 143.09, 133.89, 132.03, 129.91, 119.40, 116.77. HRMS (ESI-TOF) Calcd for C₉H₆BrO₂ [M−H]⁻: 224.9556; found: 224.9558.

### Example 62: (E)-3-(4-chlorophenyl)acrylic acid (2s)

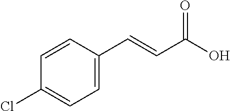 [SMILES: O=C(O)/C=C/c1ccc(Cl)cc1]

Following General Procedure on 0.1 mmol scale. Purification by reverse phase column (water:acetonitrile=10:1 to 1:4) afforded the title compound (white solid, 14.5 mg, 80% yield). ¹H NMR (500 MHz, Methanol-d₄) δ 7.62 (d, J=16.0 Hz, 1H), 7.59-7.55 (m, 2H), 7.41-7.35 (m, 2H), 6.47 (d, J=16.0 Hz, 1H). ¹³C NMR (126 MHz, Methanol-d₄) δ 168.62, 143.32, 135.73, 133.24, 129.24, 128.77, 118.86. HRMS (ESI-TOF) Calcd for C₉H₆ClO₂ [M−H]⁻: 181.0062; found: 181.0063.

### Example 63: (E)-3-(4-(methylthio)phenyl)acrylic acid (2t)

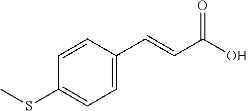

Following General Procedure on 0.1 mmol scale. Purification by reverse phase column (water:acetonitrile=10:1 to 1:4) afforded the title compound (white solid, 12.6 mg, 65% yield). ¹H NMR (500 MHz, Chloroform-d) δ 7.73 (d, J=15.9 Hz, 1H), 7.49-7.43 (m, 2H), 7.25 (d, J=5.3 Hz, 2H), 6.39 (d, J=15.9 Hz, 1H), 2.51 (s, 3H). ¹³C NMR (126 MHz, Chloroform-d) δ 172.18, 146.62, 142.82, 128.85, 127.30, 126.08, 116.17, 15.23. HRMS (ESI-TOF) Calcd for C₁₀H₉O₂S [M−H]⁻: 193.0323; found: 193.0330.

### Example 64: (E)-3-(4-((tert-butoxycarbonyl)amino)phenyl)acrylic acid (2u)

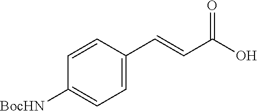 [SMILES: CC(C)(C)OC(=O)Nc1ccc(/C=C/C(=O)O)cc1]

Following General Procedure on 0.1 mmol scale. Purification by reverse phase column (water:acetonitrile=10:1 to 1:4) afforded the title compound (white solid, 11.0 mg, 42% yield). ¹H NMR (600 MHz, Methanol-d₄) δ 7.61 (d, J=15.9 Hz, 1H), 7.50 (d, J=8.8 Hz, 2H), 7.46 (d, J=8.7 Hz, 2H), 6.36 (d, J=15.9 Hz, 1H), 1.52 (s, 9H). ¹³C NMR (151 MHz, Methanol-d₄) δ 169.41, 153.47, 144.66, 141.55, 128.64, 128.53, 118.11, 115.81, 79.77, 27.25.

HRMS (ESI-TOF) Calcd for C₁₄H₁₆NO₄ [M−H]⁻: 262.1079; found: 262.1077.

### Example 65: (E)-3-(6-(trifluoromethyl)pyridin-3-yl)acrylic acid (2v)

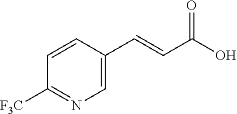 [SMILES: O=C(O)/C=C/c1ccc(C(F)(F)F)nc1]

Following General Procedure on 0.1 mmol scale. Purification by reverse phase column (water:acetonitrile=10:1 to 1:4) afforded the title compound (white solid, 16.4 mg, 76% yield). ¹H NMR (500 MHz, Methanol-d₄) δ 8.90 (d, J=2.2 Hz, 1H), 8.29 (dd, J=8.1, 2.2 Hz, 1H), 7.84 (d, J=8.2 Hz, 1H), 7.74 (d, J=16.1 Hz, 1H), 6.75 (d, J=16.1 Hz, 1H). ¹³C NMR (126 MHz, Methanol-d₄) δ 167.87, 149.35, 147.92 (q, J=34.7 Hz), 138.96, 136.23, 133.77, 123.53, 121.49 (q, J=273.1 Hz), 120.53. HRMS (ESI-TOF) Calcd for C₉H₅F₃NO₂ [M+H]⁺: 216.0272; found: 216.0273.

### Example 66: (E)-5-phenylpent-2-enoic acid (2w)

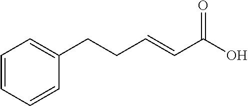 [SMILES: O=C(O)/C=C/CCc1ccccc1]

Following General Procedure on 0.1 mmol scale. Purification by reverse phase column (water:acetonitrile=10:1 to 1:4) afforded the title compound (white solid, 12.4 mg, 71% yield). ¹H NMR (600 MHz, Chloroform-d) δ 7.30 (t, J=7.6 Hz, 2H), 7.23-7.16 (m, 3H), 7.11 (dt, J=15.5, 6.9 Hz, 1H), 5.85 (dt, J=15.7, 1.6 Hz, 1H), 2.79 (t, J=7.7 Hz, 2H), 2.60-2.52 (m, 2H). ¹³C NMR (151 MHz, Chloroform-d) δ 171.40, 150.91, 140.59, 128.54, 128.34, 126.26, 121.21, 34.20, 34.00. HRMS (ESI-TOF) Calcd for C₁₁H₁₁O₂[M+H]⁺: 175.0759; found: 175.0757.

### Example 67: (E)-2-phenylbut-2-enoic acid (2x)

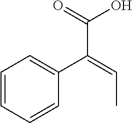

Following General Procedure on 0.1 mmol scale. Purification by reverse phase column (water:acetonitrile=10:1 to 1:4) afforded the title compound (white solid, 10.6 mg, 65% yield). ¹H NMR (500 MHz, Chloroform-d) δ 7.33-7.26 (m, 2H), 7.27-7.21 (m, 1H), 7.14-7.07 (m, 3H), 1.68 (d, J=7.0 Hz, 3H). ¹³C NMR (126 MHz, Chloroform-d) δ 172.88, 140.74, 135.49, 129.95, 128.49, 128.04, 127.34, 15.73. HRMS (ESI-TOF) Calcd for C₁₀H₉O₂ [M+H]⁺: 163.0754; found: 163.0752. Olefin geometry determined using 2D-NOE and by comparing to known literature (32).

### Example 68: (E/Z)-2-ethylbut-2-enoic acid (2y)

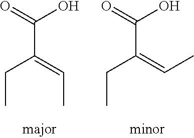

Following General Procedure on 0.1 mmol scale. Due to the volatility of the product, the yield was determined by ¹H NMR analysis of the crude product using CH₂Br₂ (0.1 mmol, 7 μL) as the internal standard (51% yield). HRMS (ESI-TOF) Calcd for C₆H₁₁O₂ [M+H]⁺: 115.0754; found: 115.0757.

Major (E-isomer): ¹H NMR (600 MHz, Chloroform-d) δ 6.98 (q, J=7.4 Hz, 1H), 2.32 (q, J=7.9 Hz, 2H), 1.86-1.80 (m, 3H), 1.02 (td, J=7.5, 2.2 Hz, 3H). ¹³C NMR (151 MHz, Chloroform-d) δ 173.55, 139.84, 134.39, 19.50, 14.36, 13.57.

Minor (Z-isomer): ¹H NMR (600 MHz, Chloroform-d) δ 6.16 (q, J=7.5 Hz, 1H), 2.28 (q, J=7.6 Hz, 2H), 2.03 (d, J=7.3 Hz, 3H), 1.06 (t, J=7.4 Hz, 3H). The olefin geometry is determined by comparing to reported data (33).

### Example 69: Methacrylic Acid (2z)

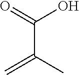

Following General Procedure on 0.1 mmol scale. Due to the volatility of the product, the yield was determined by ¹H NMR analysis of the crude product using CH₂Br₂ (0.1 mmol, 7 μL) as the internal standard (42% yield). ¹H NMR (600 MHz, Chloroform-d) δ 6.28-6.23 (m, 1H), 5.69 (t, J=1.6 Hz, 1H), 1.98-1.93 (m, 3H). ¹³C NMR (151 MHz, Chloroform-d) δ 172.87, 135.69, 127.87, 17.87. HRMS (ESI-TOF) Calcd for C₄H₅O₂ [M+H]⁺: 85.0295; found: 85.0296.

### Example 70: (Z)-2-methylhept-2-enoic acid (2aa)

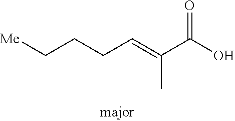

Following General Procedure on 0.1 mmol scale. Purification by reverse phase column (water:acetonitrile=10:1 to 1:4) afforded the title compound (white solid, 5.5 mg, 39% yield). ¹H NMR (600 MHz, Chloroform-d) δ 6.92 (t, J=7.5 Hz, 1H), 2.20 (q, J=7.4 Hz, 2H), 1.84 (s, 3H), 1.47-1.41 (m, 2H), 1.38-1.33 (m, 2H), 0.92 (t, J=7.3 Hz, 3H). ¹³C NMR (151 MHz, CDCl₃) δ 173.17, 145.45, 126.85, 30.55, 28.61, 22.44, 13.88, 11.99. HRMS (ESI-TOF) Calcd for C₈H₁₃O₂[M−H]⁻: 141.0916; found: 141.0916. Minor product identified by ¹H NMR spectrum.

### Example 71: 4-(tert-butyl)cyclohex-1-ene-1-carboxylic acid (2ab)

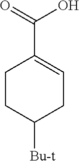

The substrate for this reaction is cis-4-(tert-butyl)cyclohexanecarboxylic acid. Following General Procedure on 0.1 mmol scale. Purification by reverse phase column (water:acetonitrile=10:1 to 1:4) afforded the title compound (white solid, 10.2 mg, 56% yield). ¹H NMR (500 MHz, Chloroform-d) δ 7.13 (dt, J=5.3, 2.5 Hz, 1H), 2.50 (ddt, J=17.6, 4.8, 2.2 Hz, 1H), 2.28 (dtt, J=19.3, 5.1, 2.2 Hz, 1H), 2.12 (dddd, J=19.9, 10.3, 5.1, 2.5 Hz, 1H), 2.03-1.86 (m, 2H), 1.32-1.26 (m, 1H), 1.14 (qd, J=12.4, 5.0 Hz, 1H), 0.89 (s, 9H). ¹³C NMR (126 MHz, Chloroform-d) δ 173.03, 143.14, 129.76, 43.33, 32.27, 27.89, 27.25, 25.36, 23.63. HRMS (ESI-TOF) Calcd for C₁₁H₁₇O₂[M−H]⁻: 181.1229; found: 181.1232.

### Example 72: cyclopent-1-ene-1-carboxylic acid (2ac)

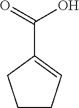

Following General Procedure on 0.1 mmol scale. Purification by reverse phase column (water:acetonitrile=10:1 to 1:4) afforded the title compound (white solid, 4.8 mg, 43% yield). ¹H NMR (600 MHz, Chloroform-d) δ 6.93 (p, J=2.3 Hz, 1H), 2.62-2.49 (m, 4H), 2.04-1.94 (m, 2H). ¹³C NMR (151 MHz, Chloroform-d) δ 170.31, 146.98, 136.09, 33.77, 31.15, 23.30. HRMS (ESI-TOF) Calcd for C₆H₉O₂ [M+H]⁺: 113.0597; found: 113.0601.

### Example 73: (R,E)-4-((3R,5R,8R,9S,10S,13R,14S,17R)-3-acetoxy-10,13-dimethylhexadecahydro-1H-cyclopenta[a]phenanthren-17-yl)pent-2-enoic acid (2ad)

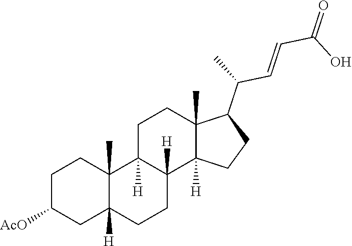

Following General Procedure on 0.1 mmol scale. Purification by reverse phase column (pure acetonitrile) afforded the title compound (white solid, 23.3 mg, 56% yield). ¹H NMR (500 MHz, Chloroform-d) δ 6.94 (dd, J=15.6, 9.0 Hz, 1H), 5.74 (d, J=15.9 Hz, 1H), 4.72 (tt, J=11.3, 4.7 Hz, 1H), 2.29 (dt, J=14.8, 6.6 Hz, 1H), 2.03 (s, 3H), 1.97-1.93 (m, 1H), 1.88-1.78 (m, 3H), 1.69 (dd, J=10.2, 5.7 Hz, 2H), 1.60-1.50 (m, 2H), 1.48-1.36 (m, 6H), 1.29-1.17 (m, 5H), 1.13-0.99 (m, 7H), 0.97-0.91 (m, 4H), 0.68 (s, 3H). ¹³C NMR (126 MHz, CDCl₃) δ 172.10, 170.71, 157.45, 118.37, 74.38, 56.30, 55.06, 43.10, 41.87, 40.47, 40.00, 39.83, 35.81, 35.05, 34.61, 32.25, 28.16, 26.99, 26.63, 26.31, 24.24, 23.33, 21.48, 20.82, 19.06, 12.30. HRMS (ESI-TOF) Calcd for C₂₆H₄₀O₄Na [M+Na]⁺: 439.2819; found: 439.2818.

### Example 74: (E)-7-oxo-7-phenylhept-2-enoic acid (2ae)

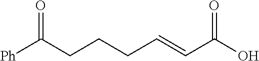 [SMILES: O=C(O)/C=C/CCCC(=O)c1ccccc1]

Following General Procedure on 0.1 mmol scale. Purification by reverse phase column (water:acetonitrile=10:1 to 1:4) afforded the title compound (white solid, 12.9 mg, 59% yield). ¹H NMR (500 MHz, Chloroform-d) δ 7.97-7.91 (m, 2H), 7.61-7.53 (m, 1H), 7.47 (dd, J=8.3, 7.2 Hz, 2H), 7.09 (dt, J=15.6, 6.9 Hz, 1H), 5.88 (dt, J=15.6, 1.6 Hz, 1H), 3.01 (t, J=7.2 Hz, 2H), 2.35 (qd, J=7.2, 1.6 Hz, 2H), 1.95 (p, J=7.3 Hz, 2H). ¹³C NMR (126 MHz, Chloroform-d) δ 199.59, 171.14, 151.16, 136.98, 133.28, 128.79, 128.14, 121.46, 37.60, 31.77, 22.36. HRMS (ESI-TOF) Calcd for C₁₃H₁₅O₃[M+H]⁺: 219.1016; found: 219.1014. Assignment supported by 2D HMBC spectrum.

### Example 75: (E)-6-oxohept-2-enoic acid (2af)

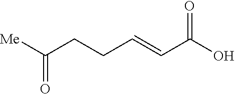

Following General Procedure on 0.1 mmol scale. Purification by reverse phase column (water:acetonitrile=10:1 to 1:4) afforded the title compound (white solid, 7.6 mg, 54% yield). ¹H NMR (600 MHz, Chloroform-d) δ 7.03 (dt, J=15.1, 6.7 Hz, 1H), 5.83 (d, J=15.6 Hz, 1H), 2.62 (t, J=7.2 Hz, 2H), 2.50 (t, J=7.1 Hz, 2H), 2.17 (d, J=1.5 Hz, 3H). ¹³C NMR (126 MHz, CDCl₃) δ 206.85, 171.79, 150.25, 121.59, 41.41, 30.06, 26.14. Assignment supported by 2D HMBC spectrum.

### Example 76: (E)-7-oxooct-2-enoic acid (2ag)

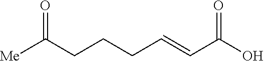 [SMILES: CC(=O)CCC/C=C/C(=O)O]

Following General Procedure on 0.1 mmol scale. Purification by reverse phase column (water:acetonitrile=10:1 to 1:4) afforded the title compound (white solid, 9.6 mg, 62% yield). ¹H NMR (500 MHz, Chloroform-d) δ 7.03 (dt, J=15.6, 7.0 Hz, 1H), 5.84 (dt, J=15.6, 1.6 Hz, 1H), 2.47 (t, J=7.3 Hz, 2H), 2.29-2.22 (m, 2H), 2.14 (s, 3H), 1.77 (p, J=7.3 Hz, 2H). ¹³C NMR (126 MHz, Chloroform-d) δ 208.06, 170.49, 150.93, 121.13, 42.52, 31.43, 30.01, 21.71. ¹³C NMR (151 MHz, Chloroform-d) δ 173.84, 173.25, 150.49, 122.17, 60.59, 33.63, 31.61, 23.31, 14.36. HRMS (ESI-TOF) Calcd for C₈H₁₁O₃⁻ [M−H]⁻: 155.0713; found: 155.0719. Assignment supported by 2D HMBC spectrum.

### Example 77: (E)-6-methoxy-6-oxohex-2-enoic acid (2ah)

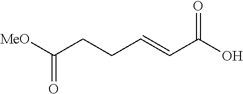 [SMILES: COC(=O)CC/C=C/C(=O)O]

Following General Procedure on 0.1 mmol scale. Purification by reverse phase column (water:acetonitrile=10:1 to 1:4) afforded the title compound (white solid, 11.8 mg, 75% yield). ¹H NMR (600 MHz, Chloroform-d) δ 7.05 (dt, J=15.7, 6.6 Hz, 1H), 5.86 (dt, J=15.6, 1.6 Hz, 1H), 3.69 (s, 3H), 2.56 (dtt, J=8.0, 6.6, 1.4 Hz, 2H), 2.50 (ddd, J=8.1, 6.7, 1.3 Hz, 2H). ¹³C NMR (151 MHz, Chloroform-d) δ 172.78, 171.57, 149.53, 121.78, 51.99, 32.22, 27.41. HRMS (ESI-TOF) Calcd for C₇H₉O₄⁻ [M−H]⁻: 157.0506; found: 157.0507. Assignment supported by 2D HMBC spectrum.

### Example 78A: (E)-7-ethoxy-7-oxohept-2-enoic acid (2ai)

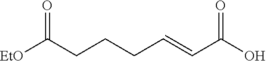 [SMILES: CCOC(=O)CCC/C=C/C(=O)O]

Following General Procedure on 0.1 mmol scale. Purification by reverse phase column (water:acetonitrile=10:1 to 1:4) afforded the title compound (white solid, 12.4 mg, 67% yield). ¹H NMR (600 MHz, Chloroform-d) δ 7.12-6.87 (m, 1H), 6.04-5.73 (m, 1H), 4.13 (q, J=7.1 Hz, 2H), 2.38-2.20 (m, 4H), 1.81 (p, J=7.4 Hz, 2H), 1.26 (t, J=7.1 Hz, 3H). ¹³C NMR (151 MHz, CDCl₃) δ 173.84, 173.25, 150.49, 122.17, 60.59, 33.63, 31.61, 23.31, 14.36. HRMS (ESI-TOF) Calcd for C₉H₁₅O₄⁺ [M+H]⁺: 187.0965; found: 187.0963.

### Example 78B: benzyl 2-(3-oxo-3a-propyl-1,3,3a,4,5,6-hexahydroisobenzofuran-1-yl)acetate (2aj)

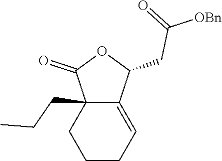 [SMILES: CCC[C@]12CCCC=C1[C@@H](CC(=O)OCc1ccccc1)OC2=O]

The reaction is conducted following the General Procedure using 1-propylcyclohexane-1-carboxylic acid as substrate, 0.8 mL HFIP is used instead of 0.8 mL of t-Amyl-OH, with the addition of 2.0 equiv. of benzyl acrylate. The product is isolated using Prep-TLC (hexane:ethyl acetate=8:1) as a white solid (14.2 mg, 41%). 1H NMR (600 MHz, Chloroform-d) δ 7.42-7.36 (m, 5H), 5.61-5.57 (m, 1H), 5.46-5.42 (m, 1H), 5.24-5.15 (m, 2H), 2.89 (dd, J=16.4, 4.6 Hz, 1H), 2.75 (dd, J=16.4, 7.5 Hz, 1H), 2.21-2.14 (m, 1H), 2.04 (dddd, J=13.9, 10.3, 6.7, 4.0 Hz, 2H), 1.75-1.62 (m, 4H), 1.50-1.38 (m, 2H), 1.33 (td, J=13.1, 4.8 Hz, 1H), 0.93 (t, J=7.3 Hz, 3H). 13C NMR (151 MHz, CDCl3) δ 178.78, 169.93, 139.72, 135.57, 128.75, 128.64, 128.56, 120.91, 67.01, 45.27, 38.40, 38.21, 29.83, 26.56, 23.76, 17.87, 16.84, 14.44. HRMS (ESI-TOF) Calcd for C20H25O4+ [M+H]+: 349.2557; found: 349.2560. Assignment supported by 2D HSQC and 2D-NOE spectrum.

### Process for Making Compounds of Formula (5) and Formula (7)

### Example 79: Ligand Effect on Butenolide Formation Reaction

The purpose of this example is to demonstrate the use of ligands (L-2) in an illustrative embodiment of the process for making a compound of formula (5) or formula (7):

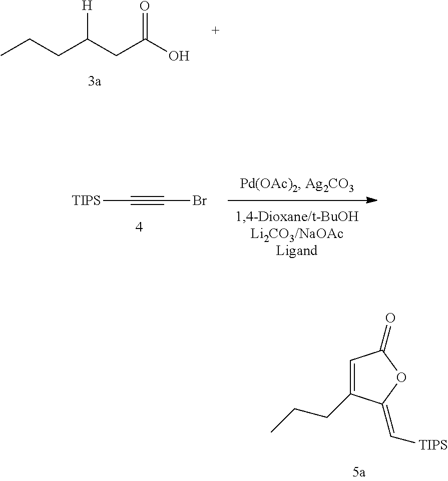

Reaction conditions were as follows: 3a (0.1 mmol), 4 (2.0 equiv), Pd(OAc)₂ (10 mol %), L (18 mol %), Ag₂CO₃ (2.0 equiv), Li₂CO₃ (2.0 equiv), NaOAc (0.5 equiv), 1,4-dioxane (0.2 mL), t-BuOH (0.8 mL), 100° C., 16 h. Yields were determined by ¹H NMR using dibromomethane as internal standard. Results are shown in Table 7 below.

TABLE 7
Ligand (L-2) Effect on Butenolide Formation Reaction.

| Ligand | Structure | Yield (%) |
|---|---|---|
| No | — | 0 |
| ligand | | |
| — | 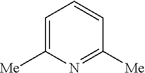 | 0 |
| — | 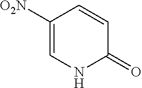 | 0 |
| L9 | 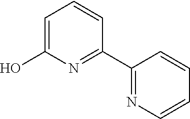 [SMILES: Oc1cccc(-c2ccccn2)n1] | 0 |
| L10 | 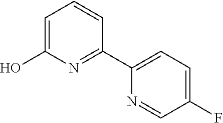 [SMILES: Oc1cccc(-c2ccc(F)cn2)n1] | 0 |
| L15 | 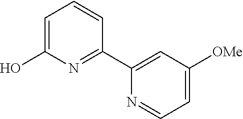 [SMILES: COc1ccnc(-c2cccc(O)n2)c1] | 0 |
| L8 | 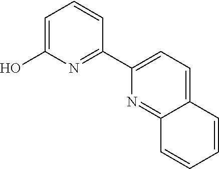 [SMILES: Oc1cccc(-c2ccc3ccccc3n2)n1] | 0 |
| L20 | 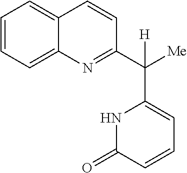 [SMILES: CC(c1ccc2ccccc2n1)c1cccc(=O)[nH]1] | <5 |
| L5 | 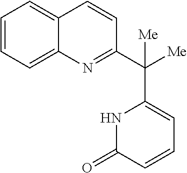 [SMILES: CC(C)(c1ccc2ccccc2n1)c1cccc(=O)[nH]1] | 38 |
| L21 | 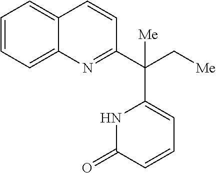 [SMILES: CCC(C)(c1ccc2ccccc2n1)c1cccc(=O)[nH]1] | 32 |
| L22 | 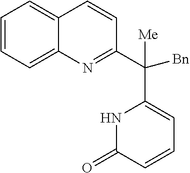 [SMILES: CC(Cc1ccccc1)(c1ccc2ccccc2n1)c1cccc(=O)[nH]1] | 54 |
| L23 | 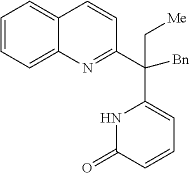 [SMILES: CCC(Cc1ccccc1)(c1ccc2ccccc2n1)c1cccc(=O)[nH]1] | 29 |
| L24 | 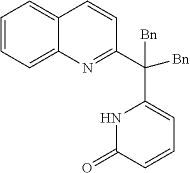 [SMILES: O=c1cccc(C(Cc2ccccc2)(Cc2ccccc2)c2ccc3ccccc3n2)[nH]1] | 21 |
| L25 | 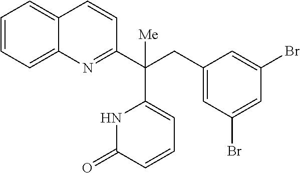 [SMILES: CC(Cc1cc(Br)cc(Br)c1)(c1ccc2ccccc2n1)c1cccc(=O)[nH]1] | 48 |
| L26 | 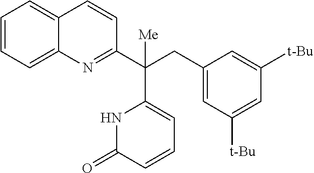 [SMILES: CC(C)(C)c1cc(CC(C)(c2ccc3ccccc3n2)c2cccc(=O)[nH]2)cc(C(C)(C)C)c1] | 51 |
| L27 | 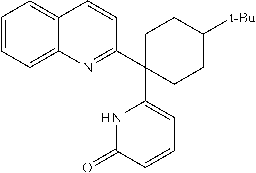 [SMILES: CC(C)(C)C1CCC(c2ccc3ccccc3n2)(c2cccc(=O)[nH]2)CC1] | 15 |
| L28 | 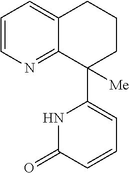 [SMILES: CC1(c2cccc(=O)[nH]2)CCCc2cccnc21] | 23 |
| L29 | 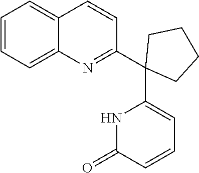 [SMILES: O=c1cccc(C2(c3ccc4ccccc4n3)CCCC2)[nH]1] | 39 |
| L30 | 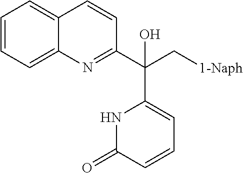 | 16 |
| L31 | 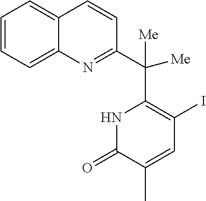 [SMILES: Cc1cc(I)c(C(C)(C)c2ccc3ccccc3n2)[nH]c1=O] | 8 |
| L32 | 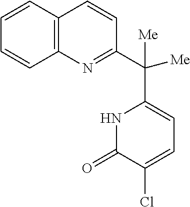 [SMILES: CC(C)(c1ccc2ccccc2n1)c1ccc(Cl)c(=O)[nH]1] | 31 |
| L33 | 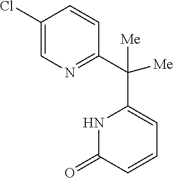 [SMILES: CC(C)(c1ccc(Cl)cn1)c1cccc(=O)[nH]1] | 68 |
| L34 | 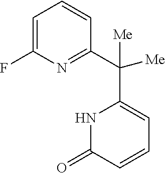 [SMILES: CC(C)(c1cccc(F)n1)c1cccc(=O)[nH]1] | 17 |
| L35 | 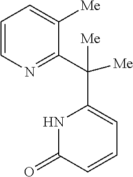 [SMILES: Cc1cccnc1C(C)(C)c1cccc(=O)[nH]1] | 41 |
| L36 | 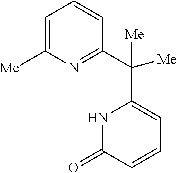 [SMILES: Cc1cccc(C(C)(C)c2cccc(=O)[nH]2)n1] | 32 |
| L37 | 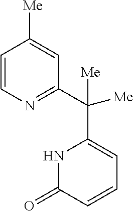 [SMILES: Cc1ccnc(C(C)(C)c2cccc(=O)[nH]2)c1] | 29 |
| L38 | 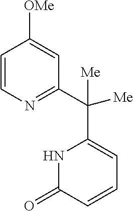 [SMILES: COc1ccnc(C(C)(C)c2cccc(=O)[nH]2)c1] | 17 |
| L39 | 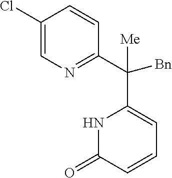 [SMILES: CC(Cc1ccccc1)(c1ccc(Cl)cn1)c1cccc(=O)[nH]1] | 57 |

### Example 80: Effect of Ag Salts and Pd Source

The purpose of this example is to illustrate use of various silver salts and sources of palladium (II) on a representative reaction:

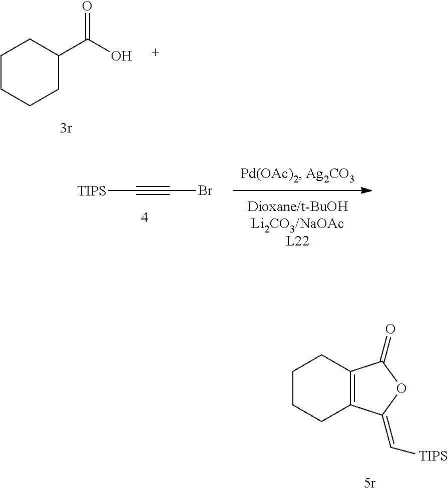

Reaction conditions were as follows: 3r (0.1 mmol), 4 (2.0 equiv), Pd(OAc)₂ (10 mol %), L22 (18 mol %), Ag₂CO₃ (2.0 equiv), Li₂CO₃ (2.0 equiv), NaOAc (0.5 equiv), Dioxane (0.2 ml), t-BuOH (0.8 mL), 100° C., 16 h. Yields determined by ¹H NMR using dibromomethane as internal standard (Table 8).

TABLE 8
Effects of Ag Salt and Pd(II) Source

| Ag Salt | Yield (%) | Pd Source | Yield (%) |
|---|---|---|---|
| Ag₂CO₃ | 52 | PdCl₂ | 34 |
| AgOAc | 36 | Pd(TFA)₂ | 21 |
| AgOPiv | 15 | Pd(MeCN)₄(BF₄)₂ | 17 |
| AgTFA | No reaction (NR) | Pd(MeCN)₂Cl₂ | 36 |
| AgF | NR | Pd(PhCN)₂Cl₂ | 30 |
| Ag₂O | 45 | [Pd(allyl)Cl]₂ | 24 |
| | | Pd(PPh₃)₂Cl₂ | 19 |

### Example 81: Effects of Solvent and Temperature

The purpose of this example is to illustrate the effects of solvent and temperature in a representative reaction:

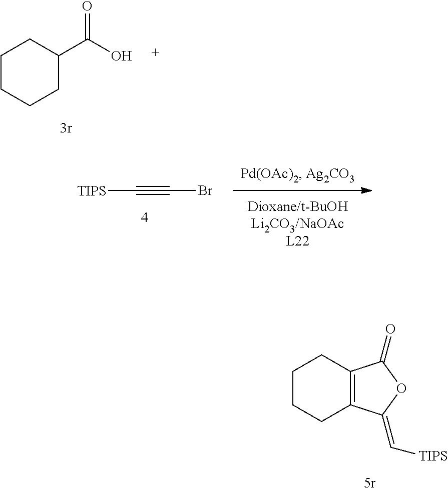

Reaction conditions were as follows: 3r (0.1 mmol), 4 (2.0 equiv), Pd(OAc)₂ (10 mol %), L22 (18 mol %), Ag₂CO₃ (2.0 equiv), Li₂CO₃ (2.0 equiv), NaOAc (0.5 equiv), solvents, 100° C., 16 h. Yields were determined by ¹H NMR using dibromomethane as internal standard (Table 9).

TABLE 9
Effects of Solvent and Temperature

| Entry | Solvent | Temperature (C.) | Yield (%) |
|---|---|---|---|
| 1 | t-BuOH | 100 | 32 |
| 2 | t-AmylOH | 100 | 30 |
| 3 | THF | 100 | 10 |
| 4 | Dioxane | 100 | 45 |
| 5 | HFIP | 100 | NR |
| 6 | Dioxane/t-BuOH (4:1) | 100 | 50 |
| 7 | Dioxane/t-BuOH (1:4) | 100 | 52 |
| 8 | Dioxane/t-BuOH (1:4) | 80 | 22 |
| 9 | Dioxane/t-BuOH (1:4) | 100 | 46 |

### Example 82: Effect of Base

The purpose of this example is to illustrate the effects of base in a representative reaction:

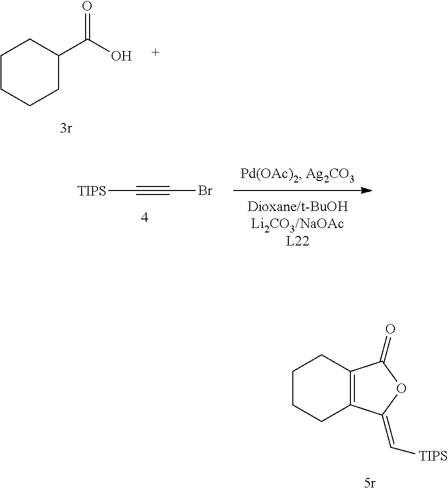

Reaction conditions were as follows: 1r (0.1 mmol), 2a (2.0 equiv), Pd(OAc)₂ (10 mol %), L10 (18 mol %), Ag₂CO₃ (2.0 equiv), Base (2.0 equiv), Dioxane (0.2 ml), t-BuOH (0.8 mL), 100° C., 16 h. Yields were determined by ¹H NMR using dibromomethane as internal standard (Table 10).

TABLE 10
Effect of Base.

| Base | Yield (%) | Base | Yield (%) |
|---|---|---|---|
| None | NR | LIF | NR |
| NaOAc | 42 | KOAc | 10 |
| NaHCO₃ | 21 | KHCO₃ | <5 |
| Na₂CO₃ | 24 | K₂CO₃ | 12 |
| NaH₂PO₄ | 20 | KH₂PO₄ | <5 |
| Na₂HPO₄ | 15 | Li₂CO₃/NaOAc (4:1) | 52 |
| Na₃PO₄ | 10 | Li₂CO₃/NaOAc (2:1) | 47 |
| Li₂CO₃ | 44 | Li₂CO₃/NaOAc (1:1) | 44 |
| LiOAc | 35 | Cs₂CO₃ | NR |
| Li₃PO₄ | 26 | CsOAc | NR |

### Example 83: Effect of Loading of Compound of Formula (3)

The purpose of this example is to demonstrate the effect on reaction yield based upon varying amounts of reagents in a representative reaction:

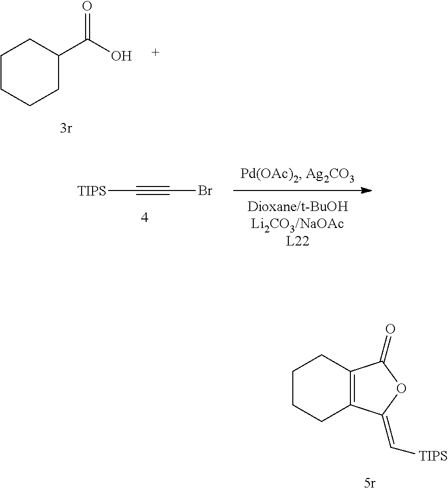

Reaction conditions were as follows: 3r (0.1 mmol), 4 (2.0 equiv), Pd(OAc)₂ (10 mol %), L22 (18 mol %), Ag₂CO₃ (2.0 equiv), Li₂CO₃ (2.0 equiv), NaOAc (0.5 equiv), Dioxane (0.2 ml), t-BuOH (0.8 mL), 100° C., 16 h. Yields determined by ¹H NMR using dibromomethane as internal standard (Table 11).

TABLE 11
Reagent Loading Effects

| Change | Yield (%) | Base | Yield (%) |
|---|---|---|---|
| None | 52 | Ag₂CO₃ (1.0 eq.) | 25 |
| Li₂CO₃/NaOAc (4:1) | 48 | Ag₂CO₃ (2.0 eq.) | 52 |
| (2.0 eq.) | | | |
| Li₂CO₃/NaOAc (4:1) | 46 | TIPS—CC—Br | 30 |
| (3.0 eq.) | | (1.0 eq.) | |
| Li₂CO₃/NaOAc (4:1) | 32 | TIPS—CC—Br | 45 |
| (4.0 eq.) | | (1.5 eq.) | |

### Example 84: Substrate Scope for Butenolide Formation Reaction

The purpose of the following examples is to demonstrate compounds of formula (5) that are made from compounds of formula (3) by the following representative procedure:

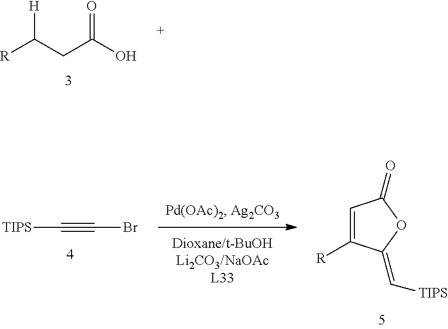

General procedure for methylene C—H activation cascade reaction: A 2-dram vial equipped with a magnetic stir bar was charged with Pd(OAc)₂ (2.2 mg, 10 mol %) and L33 (4.5 mg, 18 mol %), the appropriate carboxylic acid substrate (0.10 mmol), bromoalkyne 4 (0.2 mmol), Ag₂CO₃ (55.0 mg, 0.2 mmol), Li₂CO₃ (14.8 mg, 0.2 mmol), NaOAc (4.1 mg, 0.05 mmol) was then added in t-BuOH (0.8 mL) and 1,4-dioxane (0.2 mL) under glove box. Subsequently the vial was capped and closed tightly. The reaction mixture was then stirred at the rate of 500 rpm at room temperature for 5 minutes before it was heated under 100° C. for 16 h. After being allowed to cool to room temperature, the mixture was diluted with ethyl acetate. The mixture was passed through a pad of Celite with ethyl acetate as the eluent to remove any insoluble precipitate. The resulting solution was concentrated, and the residual mixture was dissolved with a minimal amount of acetone and loaded onto a preparative TLC plate. The pure product was then isolated using preparative TLC with ethyl acetate and hexane (1/15) as the eluent.

### Example 85: (Z)-4-propyl-5-((triisopropylsilyl)methylene)furan-2(5H)-one (5a)

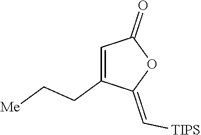

Substrate 3a was alkynylated following the general alkynylation procedure (eluent: hexane/ethyl acetate=15/1). The product was obtained as a white solid (19.9 mg, 68% yield). ¹H NMR (600 MHz, Chloroform-d) δ 5.96 (q, J=1.3 Hz, 1H), 5.28 (d, J=1.0 Hz, 1H), 2.47 (ddd, J=8.0, 7.2, 1.4 Hz, 2H), 1.67 (h, J=7.4 Hz, 2H), 1.36-1.27 (m, 3H), 1.08 (d, J=7.5 Hz, 18H), 1.02 (t, J=7.4 Hz, 3H); ¹³C NMR (151 MHz, CDCl₃) δ 170.10, 160.83, 158.66, 116.00, 105.51, 28.33, 21.49, 18.75, 13.81, 11.55. HRMS (ESI-TOF) m/z Calcd for C₁₇H₃₁O₂Si⁺ [M+H]⁺ 295.2093, found 295.2098.

### Example 86: (Z)-4-methyl-5-((triisopropylsilyl)methylene)furan-2(5H)-one (5b)

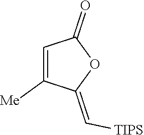

Substrate 3b was alkynylated following the general alkynylation procedure (eluent: hexane/ethyl acetate=15/1). The product was obtained as a white solid (16.0 mg, 60% yield). ¹H NMR (500 MHz, Chloroform-d) δ 5.98 (p, J=1.4 Hz, 1H), 5.27 (d, J=1.1 Hz, 1H), 2.18 (d, J=1.4 Hz, 3H), 1.31 (ddd, J=15.0, 8.0, 7.0 Hz, 3H), 1.08 (d, J=7.5 Hz, 18H); ¹³C NMR (151 MHz, CDCl₃) δ 169.90, 161.36, 154.13, 117.27, 105.71, 18.73, 12.22, 11.53. HRMS (ESI-TOF) m/z Calcd for C₁₅H₂₇O₂Si⁺ [M+H]⁺ 267.1780, found 267.1786.

### Example 87: (Z)-4-ethyl-5-((triisopropylsilyl)methylene)furan-2(5H)-one (5c)

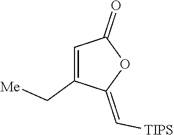

Substrate 3c was alkynylated following the general alkynylation procedure (eluent: hexane/ethyl acetate=15/1). The product was obtained as a white solid (17.4 mg, 62% yield). ¹H NMR (600 MHz, Chloroform-d) δ 6.00-5.98 (m, 1H), 5.30 (d, J=1.0 Hz, 1H), 2.55 (qd, J=7.4, 1.6 Hz, 2H), 1.33 (dt, J=15.0, 7.4 Hz, 3H), 1.28 (t, J=7.4 Hz, 3H), 1.10 (d, J=7.4 Hz, 18H); ¹³C NMR (151 MHz, CDCl₃) δ 170.08, 160.62, 160.22, 115.45, 105.22, 19.76, 18.74, 12.14, 11.54. HRMS (ESI-TOF) m/z Calcd for C₁₆H₂₉O₂Si⁺ [M+H]⁺ 281.1937, found 281.1942.

### Example 88: (Z)-4-butyl-5-((triisopropylsilyl)methylene)furan-2(5H)-one (5d)

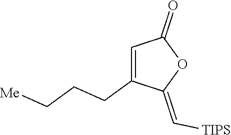

Substrate 3d was alkynylated following the general alkynylation procedure (eluent: hexane/ethyl acetate=15/1). The product was obtained as a white solid (19.4 mg, 63% yield). ¹H NMR (600 MHz, Chloroform-d) δ 5.96 (d, J=1.2 Hz, 1H), 5.28 (d, J=1.0 Hz, 1H), 2.51-2.46 (m, 2H), 1.64-1.59 (m, 2H), 1.46-1.39 (m, 2H), 1.31 (dt, J=14.9, 7.4 Hz, 3H), 1.08 (d, J=7.5 Hz, 18H), 0.97 (t, J=7.4 Hz, 3H); ¹³C NMR (151 MHz, CDCl₃) δ 170.10, 160.81, 158.91, 115.92, 105.44, 30.22, 26.05, 22.35, 18.75, 13.79, 11.55. HRMS (ESI-TOF) m/z Calcd for C₁₈H₃₃O₂Si⁺ [M+H]⁺ 309.2250, found 309.2249.

### Example 89: (Z)-4-pentyl-5-((triisopropylsilyl)methylene)furan-2(5H)-one (5e)

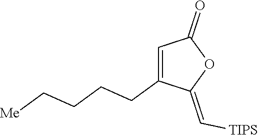

Substrate 3e was alkynylated following the general alkynylation procedure (eluent: hexane/ethyl acetate=15/1). The product was obtained as a white solid (19.3 mg, 60% yield). ¹H NMR (600 MHz, Chloroform-d) δ 5.96 (d, J=1.2 Hz, 1H), 5.28 (d, J=1.0 Hz, 1H), 2.48 (td, J=7.7, 1.5 Hz, 2H), 1.66-1.61 (m, 2H), 1.37 (ddd, J=7.2, 4.3, 3.1 Hz, 4H), 1.31 (tt, J=8.2, 7.1 Hz, 3H), 1.08 (d, J=7.5 Hz, 18H), 0.94-0.90 (m, 3H); ¹³C NMR (151 MHz, CDCl₃) δ 170.11, 160.82, 158.94, 115.93, 105.46, 31.39, 27.83, 26.32, 22.35, 18.75, 13.92, 11.55. HRMS (ESI-TOF) m/z Calcd for C₁₉H₃₅O₂Si⁺ [M+H]⁺ 323.2406, found 323.2406.

### Example 90: (Z)-5-((triisopropylsilyl)methylene)-4-undecylfuran-2(5H)-one (5f)

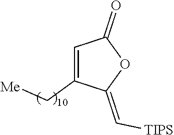

Substrate 3f was alkynylated following the general alkynylation procedure (eluent: hexane/ethyl acetate=15/1). The product was obtained as a white solid (24.0 mg, 59% yield). ¹H NMR (600 MHz, Chloroform-d) δ 5.95 (d, J=1.2 Hz, 1H), 5.28 (d, J=1.0 Hz, 1H), 2.48 (ddd, J=8.3, 7.1, 1.4 Hz, 2H), 1.65-1.59 (m, 2H), 1.41-1.36 (m, 2H), 1.34-1.25 (m, 17H), 1.08 (d, J=7.5 Hz, 18H), 0.88 (t, J=7.0 Hz, 3H); ¹³C NMR (151 MHz, CDCl₃) δ 170.12, 160.83, 158.95, 115.93, 105.47, 31.91, 29.61, 29.60, 29.48, 29.24, 28.17, 26.36, 22.69, 18.75, 14.13, 11.55. HRMS (ESI-TOF) m/z Calcd for C₂₅H₄₇O₂Si⁺ [M+H]⁺ 407.3345, found 407.3338.

### Example 91: (Z)-4-cyclopentyl-5-((triisopropylsilyl)methylene)furan-2(5H)-one (5g)

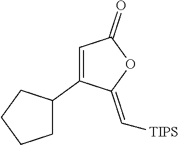

Substrate 3g was alkynylated following the general alkynylation procedure (eluent: hexane/ethyl acetate=15/1). The product was obtained as a white solid (13.4 mg, 42% yield). ¹H NMR (500 MHz, Chloroform-d) δ 5.91 (t, J=1.1 Hz, 1H), 5.32 (d, J=1.1 Hz, 1H), 2.99-2.92 (m, 1H), 2.10-2.02 (m, 2H), 1.80-1.70 (m, 3H), 1.58 (dt, J=12.3, 3.5 Hz, 3H), 1.35-1.28 (m, 3H), 1.08 (d, J=7.4 Hz, 18H); ¹³C NMR (126 MHz, CDCl₃) δ 170.24, 163.34, 160.69, 113.76, 105.95, 37.06, 33.01, 25.37, 18.76, 11.56. HRMS (ESI-TOF) m/z Calcd for C₁₉H₃₃O₂Si⁺ [M+H]⁺ 321.2250, found 321.2256.

### Example 92: (Z)-4-cyclohexyl-5-((triisopropylsilyl)methylene)furan-2(5H)-one (5h)

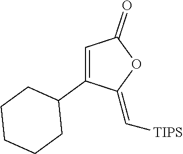

Substrate 3h was alkynylated following the general alkynylation procedure (eluent: hexane/ethyl acetate=15/1). The product was obtained as a white solid (10.3 mg, 31% yield). ¹H NMR (500 MHz, Chloroform-d) δ 5.90 (t, J=1.1 Hz, 1H), 5.29 (d, J=1.1 Hz, 1H), 2.46 (tt, J=11.8, 3.5 Hz, 1H), 1.94-1.83 (m, 4H), 1.81-1.74 (m, 1H), 1.43-1.26 (m, 8H), 1.08 (d, J=7.4 Hz, 18H); ¹³C NMR (126 MHz, CDCl₃) δ 170.28, 163.83, 159.89, 114.21, 105.40, 35.98, 33.05, 26.14, 25.86, 18.78, 11.60. HRMS (ESI-TOF) m/z Calcd for C₂₀H₃₅O₂Si⁺ [M+H]⁺ 335.2406, found 335.2410.

### Example 93: (Z)-4-benzyl-5-((triisopropylsilyl)methylene)furan-2(5H)-one (5i)

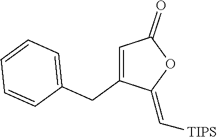

Substrate 3i was alkynylated following the general alkynylation procedure (eluent: hexane/ethyl acetate=15/1). The product was obtained as a white solid (24.0 mg, 70% yield). ¹H NMR (600 MHz, Chloroform-d) δ 7.34 (dd, J=8.1, 6.8 Hz, 2H), 7.28 (d, J=7.4 Hz, 1H), 7.22-7.19 (m, 2H), 5.81 (q, J=1.3 Hz, 1H), 5.31 (d, J=1.0 Hz, 1H), 3.83 (d, J=1.4 Hz, 2H), 1.29 (dt, J=14.8, 7.4 Hz, 3H), 1.05 (d, J=7.4 Hz, 18H); ¹³C NMR (151 MHz, CDCl₃) δ 169.60, 160.21, 157.43, 136.44, 128.93, 128.75, 127.22, 117.69, 106.83, 33.02, 18.71, 11.51. HRMS (ESI-TOF) m/z Calcd for C₂₁H₃₁O₂Si⁺ [M+H]⁺ 343.2093, found 343.2099.

### Example 94: (Z)-4-phenethyl-5-((triisopropylsilyl)methylene)furan-2(5H)-one (5j)

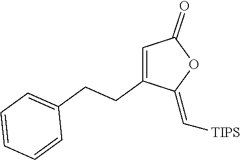

Substrate 3j was alkynylated following the general alkynylation procedure (eluent: hexane/ethyl acetate=15/1). The product was obtained as a white solid (22.8 mg, 64% yield). ¹H NMR (600 MHz, Chloroform-d) δ 7.33-7.30 (m, 2H), 7.25-7.22 (m, 1H), 7.20-7.18 (m, 2H), 5.94 (d, J=1.2 Hz, 1H), 5.27 (d, J=1.0 Hz, 1H), 2.95 (dd, J=8.5, 7.0 Hz, 2H), 2.81 (tt, J=7.8, 0.9 Hz, 2H), 1.32-1.27 (m, 3H), 1.06 (d, J=7.4 Hz, 18H); ¹³C NMR (151 MHz, CDCl₃) δ 169.87, 160.54, 157.59, 139.91, 128.22, 116.47, 116.36, 105.92, 105.83, 34.27, 28.25, 18.72, 11.55. HRMS (ESI-TOF) m/z Calcd for C₂₂H₃₃O₂Si⁺ [M+H]⁺ 357.2250, found 357.2249.

### Example 95: methyl (Z)-5-(5-oxo-2-((triisopropylsilyl)methylene)-2,5-dihydrofuran-3-yl)pentanoate (5k)

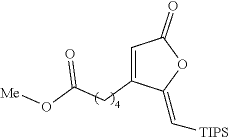

Substrate 3k was alkynylated following the general alkynylation procedure (eluent: hexane/ethyl acetate=15/1). The product was obtained as a white solid (24.5 mg, 67% yield). ¹H NMR (600 MHz, Chloroform-d) δ 5.98 (d, J=1.2 Hz, 1H), 5.28 (d, J=1.0 Hz, 1H), 3.68 (s, 3H), 2.53-2.48 (m, 2H), 2.38 (t, J=7.2 Hz, 2H), 1.76-1.71 (m, 2H), 1.70-1.65 (m, 2H), 1.31 (dt, J=14.9, 7.4 Hz, 3H), 1.07 (d, J=7.5 Hz, 18H); ¹³C NMR (151 MHz, CDCl₃) δ 173.57, 169.90, 160.62, 158.11, 116.10, 105.68, 51.65, 33.58, 27.46, 26.08, 24.48, 18.74, 11.54. HRMS (ESI-TOF) m/z Calcd for C₂₀H₃₅O₄Si⁺ [M+H]⁺ 367.2305, found 367.2303.

### Example 96: methyl (Z)-6-(5-oxo-2-((triisopropylsilyl)methylene)-2,5-dihydrofuran-3-yl)hexanoate (51)

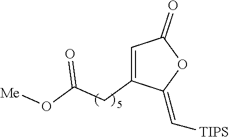

Substrate 31 was alkynylated following the general alkynylation procedure (eluent: hexane/ethyl acetate=15/1). The product was obtained as a white solid (25.1 mg, 66% yield). ¹H NMR (600 MHz, Chloroform-d) δ 5.96 (d, J=1.2 Hz, 1H), 5.27 (d, J=1.0 Hz, 1H), 3.68 (s, 3H), 2.52-2.47 (m, 2H), 2.34 (t, J=7.4 Hz, 2H), 1.66 (dq, J=19.4, 7.7 Hz, 4H), 1.47-1.40 (m, 2H), 1.35-1.28 (m, 3H), 1.07 (d, J=7.5 Hz, 18H); ¹³C NMR (151 MHz, CDCl₃) δ 173.91, 169.98, 160.70, 158.50, 116.02, 105.63, 51.58, 33.80, 28.71, 27.76, 26.14, 24.55, 18.75, 11.55. HRMS (ESI-TOF) m/z Calcd for C₂₁H₃₇O₄Si⁺ [M+H]⁺ 381.2461, found 381.2462.

### Example 97: (Z)-4-isobutyl-5-((triisopropylsilyl)methylene)furan-2(5H)-one (5m)

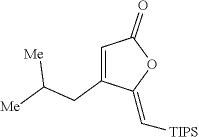

Substrate 3m was alkynylated following the general alkynylation procedure (eluent: hexane/ethyl acetate=15/1). The product was obtained as a white solid (16.0 mg, 52% yield). ¹H NMR (600 MHz, Chloroform-d) δ 5.96 (q, J=1.1 Hz, 1H), 5.29 (d, J=1.0 Hz, 1H), 2.38 (dd, J=7.1, 1.2 Hz, 2H), 1.92 (dt, J=13.5, 6.8 Hz, 1H), 1.31 (ddd, J=15.0, 7.9, 7.1 Hz, 3H), 1.08 (d, J=7.5 Hz, 18H), 0.99 (d, J=6.6 Hz, 6H); ¹³C NMR (151 MHz, CDCl₃) δ 170.08, 161.16, 157.60, 116.70, 106.06, 35.38, 28.45, 22.56, 18.75, 11.56. HRMS (ESI-TOF) m/z Calcd for C₁₈H₃₃O₂Si⁺ [M+H]⁺ 309.2250, found 309.2260.

### Example 98: (Z)-4-(cyclohexylmethyl)-5-((triisopropylsilyl)methylene)furan-2(5H)-one (5n)

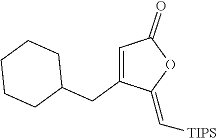

Substrate 3n was alkynylated following the general alkynylation procedure (eluent: hexane/ethyl acetate=15/1). The product was obtained as a white solid (17.1 mg, 49% yield). ¹H NMR (600 MHz, Chloroform-d) δ 5.94 (d, J=1.1 Hz, 1H), 5.28 (d, J=1.0 Hz, 1H), 2.37 (dd, J=7.0, 1.2 Hz, 2H), 1.74 (tdd, J=9.7, 5.5, 2.4 Hz, 4H), 1.69-1.64 (m, 1H), 1.56 (ddd, J=11.1, 7.5, 3.7 Hz, 1H), 1.31 (dt, J=14.9, 7.4 Hz, 3H), 1.26-1.23 (m, 1H), 1.20-1.14 (m, 2H), 1.08 (d, J=7.5 Hz, 18H), 1.00-0.95 (m, 2H); ¹³C NMR (151 MHz, CDCl₃) δ 170.10, 161.24, 157.45, 116.65, 106.05, 37.97, 34.04, 33.31, 26.18, 26.11, 18.75, 11.56. HRMS (ESI-TOF) m/z Calcd for C₂₁H₃₇O₂Si⁺ [M+H]⁺ 349.2563, found 349.2567.

### Example 99: (Z)-4-(2-cyclohexylethyl)-5-((triisopropylsilyl)methylene)furan-2(5H)-one (5o)

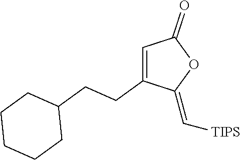

Substrate 3o was alkynylated following the general alkynylation procedure (eluent: hexane/ethyl acetate=15/1). The product was obtained as a white solid (19.2 mg, 53% yield). ¹H NMR (500 MHz, Chloroform-d) δ 5.95 (q, J=1.2 Hz, 1H), 5.28 (d, J=1.1 Hz, 1H), 2.52-2.46 (m, 2H), 1.73 (q, J=13.7, 13.3 Hz, 6H), 1.54-1.49 (m, 2H), 1.34-1.24 (m, 6H), 1.08 (d, J=7.4 Hz, 18H), 0.95 (d, J=11.4 Hz, 2H); ¹³C NMR (126 MHz, CDCl₃) δ 170.13, 160.80, 159.29, 115.83, 105.35, 37.32, 35.76, 33.16, 26.48, 26.22, 23.83, 18.75, 11.55. HRMS (ESI-TOF) m/z Calcd for C₂₂H₃₉O₂Si⁺ [M+H]⁺ 363.2719, found 363.2714.

### Example 100: (Z)-4-(4-oxo-4-phenylbutyl)-5-((triisopropylsilyl)methylene)furan-2(5H)-one (5p)

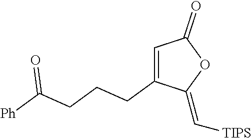

Substrate 3p was alkynylated following the general alkynylation procedure (eluent: hexane/ethyl acetate=15/1). The product was obtained as a white solid (22.3 mg, 56% yield). ¹H NMR (600 MHz, Chloroform-d) δ 7.97-7.94 (m, 2H), 7.60-7.56 (m, 1H), 7.50-7.46 (m, 2H), 6.02 (d, J=1.3 Hz, 1H), 5.35 (d, J=1.0 Hz, 1H), 3.10 (t, J=6.8 Hz, 2H), 2.65-2.57 (m, 2H), 2.10 (dq, J=8.4, 6.9 Hz, 2H), 1.30 (dt, J=14.9, 7.5 Hz, 3H), 1.07 (d, J=7.4 Hz, 18H); ¹³C NMR (151 MHz, CDCl₃) δ 198.90, 169.90, 160.56, 158.07, 136.66, 133.34, 128.72, 127.98, 116.24, 106.07, 37.26, 25.75, 22.35, 18.75, 11.54. HRMS (ESI-TOF) m/z Calcd for C₂₄H₃₅O₃Si⁺ [M+H]⁺ 399.2355, found 399.2355.

### Example 101: (Z)-4-((1-tosylpiperidin-4-yl)methyl)-5-((triisopropylsilyl)methylene)furan-2(5H)-one (5q)

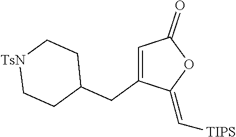

Substrate 3q was alkynylated following the general alkynylation procedure (eluent: hexane/ethyl acetate=15/1). The product was obtained as a white solid (23.1 mg, 46% yield). ¹H NMR (600 MHz, Chloroform-d) δ 7.66-7.62 (m, 2H), 7.33 (dt, J=8.0, 0.8 Hz, 2H), 5.90 (q, J=1.0 Hz, 1H), 5.25 (d, J=1.0 Hz, 1H), 3.81 (dt, J=11.5, 2.5 Hz, 2H), 2.44 (s, 3H), 2.41 (dd, J=6.9, 1.2 Hz, 2H), 2.23 (td, J=11.9, 2.5 Hz, 2H), 1.79-1.74 (m, 2H), 1.51-1.45 (m, 1H), 1.41 (td, J=12.5, 4.0 Hz, 2H), 1.30 (ddd, J=14.8, 7.9, 7.0 Hz, 3H), 1.06 (d, J=7.5 Hz, 18H); ¹³C NMR (151 MHz, CDCl₃) δ 169.54, 160.82, 155.94, 143.62, 133.04, 129.68, 116.89, 106.71, 46.17, 35.47, 32.61, 31.51, 30.95, 21.54, 18.74, 11.52. HRMS (ESI-TOF) m/z Calcd for C₂₇H₄₂NO₄SSi⁺ [M+H]⁺ 504.2604, found 504.2599.

### Example 102: (Z)-4-pentadecyl-5-((triisopropylsilyl)methylene)furan-2(51)-one (5r)

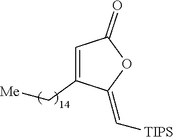

Substrate 3x was alkynylated following the general alkynylation procedure (eluent: hexane/ethyl acetate=15/1). The product was obtained as a white solid (25.0 mg, 54% yield). ¹H NMR (600 MHz, Chloroform-d) δ 5.95 (d, J=1.2 Hz, 1H), 5.28 (d, J=1.0 Hz, 1H), 2.50-2.44 (m, 2H), 1.62 (p, J=7.6 Hz, 2H), 1.38 (td, J=8.5, 8.0, 4.8 Hz, 2H), 1.33-1.25 (m, 25H), 1.09-1.06 (m, 18H), 0.90-0.86 (m, 3H); ¹³C NMR (151 MHz, CDCl₃) δ 170.12, 160.83, 158.96, 115.93, 105.47, 31.94, 29.70, 29.70, 29.68, 29.66, 29.60, 29.49, 29.37, 29.30, 29.24, 28.17, 26.36, 22.71, 18.75, 14.14, 11.55. HRMS (ESI-TOF) m z Calcd for C₂₉H₅₅O₂Si⁺ [M+H]⁺ 463.3971, found 463.3969.

### Example 103: tert-butyl (Z)-15-(5-oxo-2-((triisopropylsilyl)methylene)-2,5-dihydrofuran-3-yl)pentadecanoate (5s)

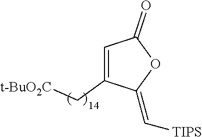

Substrate 3y was alkynylated following the general alkynylation procedure (eluent: hexane/ethyl acetate=15/1). The product was obtained as a white solid (25.8 mg, 47% yield). ¹H NMR (600 MHz, Chloroform-d) δ 5.96 (d, J=4.2 Hz, 1H), 5.28 (d, J=3.4 Hz, 1H), 2.48 (q, J=7.6, 5.9 Hz, 2H), 2.20 (t, J=7.5 Hz, 2H), 1.64-1.55 (m, 6H), 1.44 (s, 9H), 1.32-1.25 (m, 21H), 1.08 (d, J=7.5 Hz, 18H); ¹³C NMR (151 MHz, CDCl₃) δ 173.38, 170.12, 160.83, 158.96, 115.93, 105.46, 79.90, 35.64, 29.64, 29.63, 29.61, 29.60, 29.49, 29.32, 29.31, 29.25, 29.11, 28.17, 28.13, 26.36, 25.12, 18.75, 11.55. HRMS (ESI-TOF) m z Calcd for C₃₃H₆₁O₄Si⁺ [M+H]⁺ 549.4339, found 549.4341.

### Example 104: (Z)-3-((triisopropylsilyl)methylene)-4,5,6,7-tetrahydroisobenzofuran-1(3H)-one (5t)

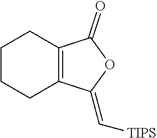

Substrate 3r was alkynylated following the general alkynylation procedure (eluent: hexane/ethyl acetate=15/1). The product was obtained as a white solid (19.6 mg, 64% yield). ¹H NMR (600 MHz, Chloroform-d) δ 5.04 (s, 1H), 2.42 (tt, J=6.1, 2.3 Hz, 2H), 2.32 (td, J=5.8, 2.7 Hz, 2H), 1.82-1.73 (m, 4H), 1.30 (dt, J=14.9, 7.4 Hz, 3H), 1.07 (d, J=7.5 Hz, 18H); ¹³C NMR (151 MHz, CDCl₃) δ 170.44, 159.96, 150.75, 128.44, 102.45, 21.55, 21.52, 21.34, 20.05, 18.76, 11.57. HRMS (ESI-TOF) m/z Calcd for C₁₈H₃₁O₂Si⁺ [M+H]⁺ 307.2093, found 307.2095. The Z/E isomer assignment is supported by 2D-NOE spectrum.

### Example 105: (Z)-6-methoxy-3-((triisopropylsilyl)methylene)-4,5,6,7-tetrahydroisobenzofuran-1(3H)-one (5u)

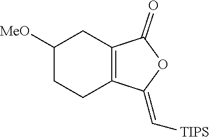

Substrate 3s was alkynylated following the general alkynylation procedure (eluent: hexane/ethyl acetate=15/1). The product was obtained as a white solid (20.5 mg, 61% yield). ¹H NMR (600 MHz, Chloroform-d) δ 5.08 (s, 1H), 3.72 (ddd, J=7.3, 4.8, 2.6 Hz, 1H), 3.39 (s, 3H), 2.58 (dtd, J=16.0, 4.2, 2.6 Hz, 2H), 2.45 (dddd, J=16.1, 8.3, 4.5, 1.8 Hz, 2H), 2.05-1.99 (m, 1H), 1.90-1.85 (m, 1H), 1.30 (dt, J=14.9, 7.5 Hz, 3H), 1.07 (dd, J=7.6, 1.2 Hz, 18H); ¹³C NMR (151 MHz, CDCl₃) δ 170.08, 159.42, 150.29, 126.04, 103.46, 73.90, 56.25, 26.05, 25.68, 18.74, 18.42, 11.56. HRMS (ESI-TOF) m/z Calcd for C₁₉H₃₃O₃Si⁺ [M+H]⁺ 337.2199, found 337.2202.

### Example 106: (Z)-5-(4-chlorophenyl)-3-((triisopropylsilyl)methylene)-4,5,6,7-tetrahydroisobenzofuran-1(3H)-one (5v)

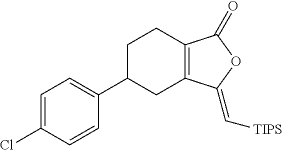

Substrate 3t was alkynylated following the general alkynylation procedure (eluent: hexane/ethyl acetate=15/1). The product was obtained as a white solid (25.8 mg, 62% yield). ¹H NMR (600 MHz, Chloroform-d) δ 7.32 (d, J=8.4 Hz, 2H), 7.19 (d, J=8.4 Hz, 2H), 5.07 (s, 1H), 2.93 (dddd, J=13.1, 11.1, 5.1, 2.7 Hz, 1H), 2.81-2.73 (m, 1H), 2.57 (ddt, J=20.6, 4.7, 2.0 Hz, 1H), 2.50-2.36 (m, 2H), 2.15-2.06 (m, 1H), 1.82 (dddd, J=13.3, 12.2, 10.8, 5.5 Hz, 1H), 1.35-1.24 (m, 3H), 1.07 (d, J=7.6 Hz, 18H); ¹³C NMR (151 MHz, CDCl₃) δ 169.87, 159.31, 150.27, 143.27, 132.51, 128.89, 128.20, 128.18, 103.32, 39.24, 29.40, 29.11, 20.66, 18.75, 11.55. HRMS (ESI-TOF) m/z Calcd for C₂₄H₃₄ClO₂Si⁺ [M+H]⁺ 417.2017, found 417.2018.

### Example 107: (Z)-3-((triisopropylsilyl)methylene)-3,4,5,6,7,8-hexahydro-1H-cyclohepta[c]furan-1-one (5w)

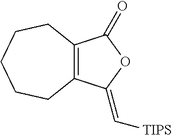

Substrate 3u was alkynylated following the general alkynylation procedure (eluent: hexane/ethyl acetate=15/1). The product was obtained as a white solid (18.2 mg, 57% yield). ¹H NMR (600 MHz, Chloroform-d) δ 5.20 (s, 1H), 2.58-2.54 (m, 2H), 2.52-2.47 (m, 2H), 1.83 (qd, J=5.7, 4.5, 1.5 Hz, 2H), 1.72 (dd, J=7.9, 3.4 Hz, 2H), 1.66 (td, J=6.8, 6.1, 3.6 Hz, 2H), 1.33-1.28 (m, 3H), 1.07 (d, J=7.5 Hz, 18H); ¹³C NMR (151 MHz, CDCl₃) δ 171.02, 159.92, 152.29, 130.81, 102.78, 30.65, 29.72, 26.62, 26.41, 24.74, 18.79, 11.60. HRMS (ESI-TOF) m/z Calcd for C₁₉H₃₃O₂Si⁺ [M+H]⁺ 321.2250, found 321.2251.

### Example 108: (Z)-3-((triisopropylsilyl)methylene)-6,7-dihydro-3H-furo[3,4-c]pyran-1(4H)-one (5x)

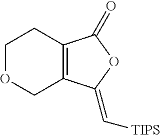

Substrate 3v was alkynylated following the general alkynylation procedure (eluent: hexane/ethyl acetate=15/1). The product was obtained as a white solid (18.2 mg, 59% yield). ¹H NMR (600 MHz, Chloroform-d) δ 4.95 (s, 1H), 4.56 (t, J=2.4 Hz, 2H), 3.88 (t, J=5.4 Hz, 2H), 2.47 (dqd, J=5.4, 2.4, 1.2 Hz, 2H), 1.30 (ddd, J=14.8, 7.9, 7.1 Hz, 3H), 1.07 (d, J=7.5 Hz, 18H); ¹³C NMR (151 MHz, CDCl₃) δ 168.93, 156.68, 149.15, 126.33, 105.16, 63.74, 62.19, 20.91, 18.72, 11.55. HRMS (ESI-TOF) m/z Calcd for C₁₇H₂₉O₃Si⁺ [M+H]⁺ 309.1886, found 309.1889.

### Example 109: (Z)-5-tosyl-3-((triisopropylsilyl)methylene)-4,5,6,7-tetrahydrofuro[3,4-c]pyridin-1(3H)-one (5y)

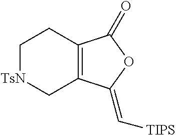

Substrate 3w was alkynylated following the general alkynylation procedure (eluent: hexane/ethyl acetate=15/1). The product was obtained as a white solid (20.4 mg, 48% yield). ¹H NMR (500 MHz, Chloroform-d) δ 7.73 (d, J=8.3 Hz, 2H), 7.36 (d, J=7.9 Hz, 2H), 5.06 (s, 1H), 4.10 (d, J=2.4 Hz, 2H), 3.36 (t, J=5.7 Hz, 2H), 2.52-2.48 (m, 2H), 2.44 (s, 3H), 1.31-1.26 (m, 3H), 1.06 (d, J=7.4 Hz, 18H); ¹³C NMR (151 MHz, CDCl₃) δ 168.23, 156.62, 145.80, 144.35, 133.51, 130.05, 127.51, 126.77, 105.67, 42.52, 41.98, 21.58, 20.75, 18.71, 11.54. HRMS (ESI-TOF) m/z Calcd for C₂₄H₃₆NO₄SSi⁺ [M+H]⁺ 426.2134, found 426.2143.

### Example 110: (Z)-5-((tert-butyldiphenylsilyl)methylene)-4-propylfuran-2(5H)-one (5z)

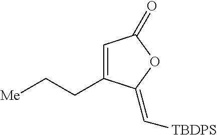

Substrate 3a was alkynylated following the general alkynylation procedure (eluent: hexane/ethyl acetate=15/1). The product was obtained as a white solid (17.7 mg, 47% yield). ¹H NMR (600 MHz, Chloroform-d) δ 7.63 (dt, J=6.8, 1.5 Hz, 4H), 7.41-7.38 (m, 2H), 7.37-7.33 (m, 4H), 5.97 (d, J=1.2 Hz, 1H), 5.65 (d, J=1.0 Hz, 1H), 2.53 (ddd, J=8.0, 7.2, 1.4 Hz, 2H), 1.71 (h, J=7.4 Hz, 2H), 1.13 (s, 9H), 1.05 (t, J=7.4 Hz, 3H); ¹³C NMR (151 MHz, CDCl₃) δ 169.34, 161.67, 158.73, 135.92, 133.72, 129.42, 127.70, 116.67, 103.52, 28.34, 27.77, 21.37, 18.53, 13.86. HRMS (ESI-TOF) m/z Calcd for C₂₄H₂₉O₂Si⁺ [M+H]⁺ 377.1937, found 377.1941.

### Example 111: (Z)-5-((1-((tert-butyldimethylsilyl)oxy)cyclohexyl)methylene)-4-propylfuran-2(5H)-one (5aa)

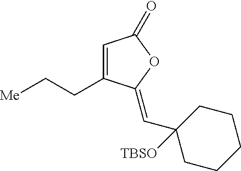 [SMILES: CCCC1=CC(=O)O/C1=C\C1(O[Si](C)(C)C(C)(C)C)CCCCC1]

Substrate 3a was alkynylated following the general alkynylation procedure (eluent: hexane/ethyl acetate=15/1). The product was obtained as a white solid (14.7 mg, 42% yield). ¹H NMR (600 MHz, Chloroform-d) δ 5.92-5.88 (m, 1H), 5.29 (s, 1H), 2.42-2.37 (m, 2H), 1.90-1.85 (m, 3H), 1.79 (d, J=13.4 Hz, 3H), 1.69-1.65 (m, 6H), 1.02 (dd, J=8.0, 6.7 Hz, 3H), 0.90 (s, 9H), 0.05 (s, 6H); ¹³C NMR (151 MHz, CDCl₃) δ 169.48, 160.57, 129.78, 118.94, 114.73, 76.81, 74.04, 40.77, 38.58, 28.14, 25.89, 25.81, 25.64, 25.37, 22.16, 21.34, 18.35, 13.81. HRMS (ESI-TOF) m/z Calcd for C₁₄H₁₉O₂ [M-OTBS]⁺ 219.1380, found 219.1384.

### Example 112: Substrate Scope of the Methyl C—H Activation Cascade Reaction

The purpose of the following examples is to demonstrate compounds of formula (7) that are made from compounds of formula (6) by the following representative procedure:

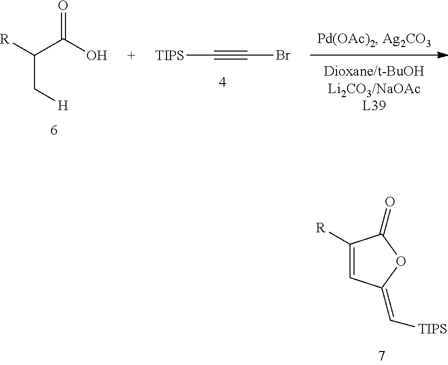

General procedure for methyl C—H activation cascade reaction: A 2-dram vial equipped with a magnetic stir bar was charged with Pd(OAc)₂ (2.2 mg, 10 mol %) and L39 (5.8 mg, 18 mol %). Then the appropriate carboxylic acid substrate (0.10 mmol), bromoalkyne 4 (0.2 mmol), Ag₂CO₃ (55.0 mg, 0.2 mmol), Li₂CO₃ (14.8 mg, 0.2 mmol), NaOAc (4.1 mg, 0.05 mmol), t-BuOH (0.8 mL), and 1,4-dioxane (0.2 mL) were then added in the glove box. Subsequently the vial was capped and closed tightly. The reaction mixture was then stirred at the rate of 500 rpm at room temperature for 5 minutes before it was heated under 100° C. for 16 h. After being allowed to cool to room temperature, the mixture was diluted with ethyl acetate. The mixture was passed through a pad of Celite with ethyl acetate as the eluent to remove any insoluble precipitate. The resulting solution was concentrated, and the residual mixture was dissolved with a minimal amount of acetone and loaded onto a preparative TLC plate. The pure product was then isolated using preparative TLC with ethyl acetate and hexane (1/15) as the eluent.

### Example 112: (Z)-3-methyl-5-((triisopropylsilyl)methylene)furan-2(5H)-one (7a)

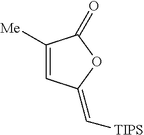

Substrate 6a was alkynylated following the general alkynylation procedure (eluent: hexane/ethyl acetate=15/1). The product was obtained as a white solid (21.8 mg, 82% yield). ¹H NMR (600 MHz, Chloroform-d) δ 6.95 (d, J=1.6 Hz, 1H), 5.11 (s, 1H), 2.02-1.99 (m, 3H), 1.33-1.24 (m, 3H), 1.07 (d, J=7.5 Hz, 18H); ¹³C NMR (126 MHz, CDCl₃) δ 171.79, 158.77, 138.22, 130.92, 108.05, 18.72, 11.54, 10.45. HRMS (ESI-TOF) m/z Calcd for C₁₅H₂₇O₂Si⁺ [M+H]⁺ 267.1780, found 267.1783.

### Example 113: (Z)-3-ethyl-5-((triisopropylsilyl)methylene)furan-2(5H)-one (7b)

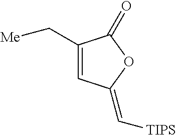

Substrate 6b was alkynylated following the general alkynylation procedure (eluent: hexane/ethyl acetate=15/1). The product was obtained as a white solid (15.4 mg, 55% yield). ¹H NMR (600 MHz, Chloroform-d) δ 6.93 (t, J=1.7 Hz, 1H), 5.12 (d, J=0.6 Hz, 1H), 2.40 (qd, J=7.5, 1.7 Hz, 2H), 1.29 (ddd, J=14.9, 7.8, 6.7 Hz, 3H), 1.21 (t, J=7.4 Hz, 3H), 1.07 (d, J=7.5 Hz, 18H); ¹³C NMR (151 MHz, CDCl₃) δ 171.30, 158.97, 136.95, 136.69, 107.99, 18.73, 18.54, 11.76, 11.54. HRMS (ESI-TOF) m/z Calcd for C₁₅H₂₉O₂Si⁺ [M+H]⁺ 281.1937, found 281.1940.

### Example 114: (Z)-3,4-dimethyl-5-((triisopropylsilyl)methylene)furan-2(5H)-one (7b′)

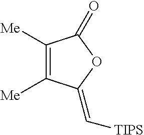

Substrate 6b was alkynylated following the general alkynylation procedure (eluent: hexane/ethyl acetate=15/1). The product was obtained as a white solid (4.7 mg, 17% yield). ¹H NMR (600 MHz, Chloroform-d) δ 5.15 (s, 1H), 2.06 (d, J=1.1 Hz, 3H), 1.92 (s, 3H), 1.33-1.27 (m, 3H), 1.07 (d, J=7.5 Hz, 18H); ¹³C NMR (151 MHz, CDCl₃) δ 171.37, 160.79, 146.50, 125.54, 102.69, 18.76, 11.58, 10.33, 8.85. HRMS (ESI-TOF) m/z Calcd for C₁₅H₂₉O₂Si⁺ [M+H]⁺ 281.1937, found 281.1940

### Example 115: (Z)-3-(tert-butyl)-5-((triisopropylsilyl)methylene)furan-2(5H)-one (7c)

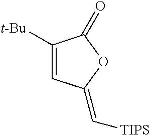

Substrate 6c was alkynylated following the general alkynylation procedure (eluent: hexane/ethyl acetate=15/1). The product was obtained as a white solid (19.7 mg, 64% yield). ¹H NMR (600 MHz, Chloroform-d) δ 6.87 (s, 1H), 5.08 (s, 1H), 1.25-1.30 (m, 12H), 1.07 (d, J=7.5 Hz, 18H); ¹³C NMR (151 MHz, CDCl₃) δ 169.49, 158.35, 143.46, 135.51, 107.71, 31.65, 28.27, 18.75, 11.54. HRMS (ESI-TOF) m/z Calcd for C₁₈H₃₃O₂Si⁺ [M+H]⁺ 309.2250, found 309.2255.

### Example 116: (Z)-3-(5-chloropentyl)-5-((triisopropylsilyl)methylene)furan-2(5H)-one (7d)

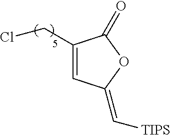

Substrate 6d was alkynylated following the general alkynylation procedure (eluent: hexane/ethyl acetate=15/1). The major product was obtained as a white solid (12.1 mg, 34% yield). ¹H NMR (600 MHz, Chloroform-d) δ 6.94 (t, J=1.5 Hz, 1H), 5.13 (s, 1H), 3.55 (t, J=6.6 Hz, 2H), 2.42-2.36 (m, 2H), 1.85-1.79 (m, 2H), 1.66-1.60 (m, 2H), 1.54-1.49 (m, 2H), 1.32-1.27 (m, 3H), 1.07 (d, J=7.5 Hz, 18H); ¹³C NMR (151 MHz, CDCl₃) δ 171.33, 158.82, 137.41, 135.02, 108.45, 44.81, 32.17, 26.80, 26.46, 24.98, 18.73, 11.54. HRMS (ESI-TOF) m/z Calcd for C₁₉H₃₄ClO₂Si⁺ [M+H]⁺ 357.2017, found 357.2018.

### Example 117: (Z)-3-(4-chlorobenzyl)-5-((triisopropylsilyl)methylene)furan-2(5H)-one (7e)

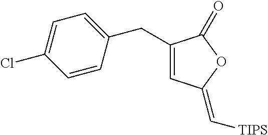

Substrate 6e was alkynylated following the general alkynylation procedure (eluent: hexane/ethyl acetate=15/1). The major product was obtained as a white solid. (23.3 mg, 62% yield). ¹H NMR (600 MHz, Chloroform-d) δ 7.31 (d, J=8.4 Hz, 2H), 7.19 (d, J=8.4 Hz, 2H), 6.73 (t, J=1.7 Hz, 1H), 5.13 (s, 1H), 3.65 (d, J=1.5 Hz, 2H), 1.32-1.24 (m, 3H), 1.05 (d, J=7.5 Hz, 18H); ¹³C NMR (151 MHz, CDCl₃) δ 170.75, 158.58, 138.39, 135.48, 134.37, 132.90, 130.37, 129.00, 109.83, 30.99, 18.70, 11.51. HRMS (ESI-TOF) m z Calcd for C₂₁H₃₀ClO₂Si⁺ [M+H]⁺ 377.1704, found 377.1697.

### Example 118: (Z)-3-(4-fluorophenethyl)-5-((triisopropylsilyl)methylene)furan-2(5H)-one (7f)

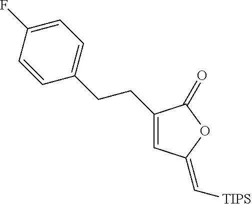

Substrate 6f was alkynylated following the general alkynylation procedure (eluent: hexane/ethyl acetate=15/1). The major product was obtained as a white solid (18.0 mg, 48% yield). ¹H NMR (600 MHz, Chloroform-d) δ 7.16-7.12 (m, 2H), 7.00-6.96 (m, 2H), 6.83 (t, J=1.4 Hz, 1H), 5.12 (s, 1H), 2.89 (t, J=7.8 Hz, 2H), 2.68 (dd, J=8.2, 1.4 Hz, 2H), 1.32-1.26 (m, 3H), 1.07 (d, J=7.4 Hz, 18H); ¹³C NMR (151 MHz, CDCl₃) δ 171.16, 161.52 (d, J=243.00 Hz), 158.68, 137.92, 136.06 (d, J=3.00 Hz), 134.03, 129.74 (d, J=7.50 Hz), 115.32 (d, J=21.00 Hz), 108.93, 32.75, 26.95, 18.72, 11.53; ¹⁹F NMR (376 MHz, CDCl₃) δ −119.50. HRMS (ESI-TOF) m/z Calcd for C₂₂H₃₂FO₂Si⁺ [M+H]⁺ 375.2156, found 375.2160.

### Example 119: (Z)-3-(3-phenylpropyl)-5-((triisopropylsilyl)methylene)furan-2(5H)-one (7g)

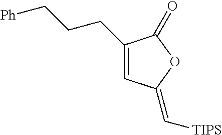

Substrate 6g was alkynylated following the general alkynylation procedure (eluent: hexane/ethyl acetate=15/1). The major product was obtained as a white solid. (15.5 mg, 42% yield). ¹H NMR (500 MHz, Chloroform-d) δ 7.29 (t, J=7.5 Hz, 2H), 7.19 (d, J=7.5 Hz, 3H), 6.91 (d, J=1.5 Hz, 1H), 5.11 (s, 1H), 2.69 (t, J=7.6 Hz, 2H), 2.40 (t, J=7.8 Hz, 2H), 1.93 (p, J=7.7 Hz, 2H), 1.29 (dt, J=15.1, 7.6 Hz, 3H), 1.06 (d, J=7.4 Hz, 18H); ¹³C NMR (126 MHz, CDCl₃) δ 171.30, 158.86, 141.41, 137.35, 135.12, 128.45, 128.43, 126.05, 108.32, 35.43, 29.07, 24.69, 18.73, 11.54. HRMS (ESI-TOF) m/z Calcd for C₂₃H₃₄O₂Si⁺ [M+H]⁺ 371.2406, found 371.2413.

### Example 120: (Z)-3-(2-phenoxyethyl)-5-((triisopropylsilyl)methylene)furan-2(5H)-one (7h)

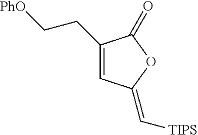

Substrate 6h was alkynylated following the general alkynylation procedure (eluent: hexane/ethyl acetate=15/1). The major product was obtained as a white solid. (14.9 mg, 40% yield). ¹H NMR (600 MHz, Chloroform-d) δ 7.30 (dd, J=8.3, 7.0 Hz, 2H), 7.23-7.17 (m, 3H), 6.84 (t, J=1.3 Hz, 1H), 5.10 (s, 1H), 2.92 (t, J=7.8 Hz, 2H), 2.70 (ddd, J=8.9, 7.0, 1.4 Hz, 2H), 1.29 (dt, J=14.9, 7.4 Hz, 3H), 1.06 (d, J=7.5 Hz, 18H); ¹³C NMR (151 MHz, CDCl₃) δ 171.24, 158.77, 140.44, 137.83, 134.34, 128.54, 128.35, 126.35, 108.66, 33.53, 26.76, 18.72, 11.53. HRMS (ESI-TOF) m/z Calcd for C₂₂H₃₃O₃Si⁺ [M+H]⁺ 373.2199, found 373.2187.

### Example 121: (Z)-4-(2-oxo-5-((triisopropylsilyl)methylene)-2,5-dihydrofuran-3-yl)butyl benzoate (7i)

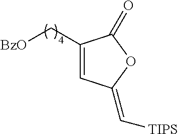

Substrate 6i was alkynylated following the general alkynylation procedure (eluent: hexane/ethyl acetate=15/1). The major product was obtained as a mixture of Z/E isomers. (16.3 mg, 38% yield). ¹H NMR (600 MHz, Chloroform-d) δ 8.08-8.00 (m, 2H), 7.58-7.53 (m, 1H), 7.48-7.41 (m, 2H), 6.97-6.95 (m, 1H), 5.13 (s, 1H), 4.36 (t, J=6.4 Hz, 2H), 2.52-2.42 (m, 2H), 1.88-1.81 (m, 2H), 1.80-1.73 (m, 2H), 1.33-1.26 (m, 3H), 1.07 (d, J=7.5 Hz, 18H). ¹³C NMR (151 MHz, CDCl₃) δ 171.26, 166.62, 158.78, 137.55, 134.81, 132.96, 129.56, 128.39, 108.64, 64.45, 28.42, 24.82, 24.18, 18.73, 11.54. HRMS (ESI-TOF) m/z Calcd for C₂₅H₃₇O₄Si⁺ [M+H]⁺ 429.2461, found 429.2459.

### Example 122: tert-butyl (Z)-3-((2-oxo-5-((triisopropylsilyl)methylene)-2,5-dihydrofuran-3-yl)methyl)pyrrolidine-1-carboxylate (7j)

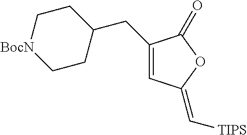

Substrate 6j was alkynylated following the general alkynylation procedure (eluent: hexane/ethyl acetate=15/1). The major product was obtained as a mixture of Z/E isomers. (26.5 mg, 61% yield). ¹H NMR (600 MHz, Chloroform-d) δ 6.94 (t, J=1.0 Hz, 1H), 5.15 (s, 1H), 4.09 (s, 2H), 2.69 (s, 2H), 2.32 (d, J=7.0 Hz, 2H), 1.79 (th, J=11.0, 3.6 Hz, 1H), 1.67 (d, J=13.5 Hz, 2H), 1.60-1.52 (m, 2H), 1.45 (s, 9H), 1.29 (dt, J=15.0, 7.5 Hz, 3H), 1.07 (d, J=7.5 Hz, 18H); ¹³C NMR (151 MHz, CDCl₃) δ 171.37, 158.61, 154.82, 138.71, 132.89, 108.96, 79.40, 34.72, 31.99, 28.46, 18.74, 18.56, 11.53. HRMS (ESI-TOF) m/z Calcd for C₂₄H₄₂NO₄Si⁺ [M+H]⁺ 436.2883, found 436.2880.

### Application to Bioactive Products

Because butenolide natural products are often bioactive, the process disclosed herein for the construction of γ-alkylidene butenolides from aliphatic carboxylic acids can allow late-stage introduction of butenolide moieties into complex natural products and drug molecules, and thus provide access to hitherto unknown hybrid molecules with potential biological activities. To illustrate such introduction, as shown in the examples below, the anti-asthmatic drug seratrodast was subjected to the standard reaction conditions and the corresponding butenolide hybrid 5ab was obtained in 42% yield. The success with two more examples 71 and 7m further demonstrates the compatibility of this reaction with complex molecules.

### Example 123: (Z)-2,3,5-trimethyl-6-(4-(5-oxo-2-((triisopropylsilyl)methylene)-2,5-dihydrofuran-3-yl)-1-phenylbutyl)cyclohexa-2,5-diene-1,4-dione (5ab)

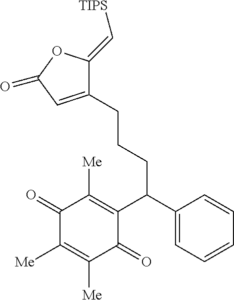

Substrate 3ab was alkynylated following the general alkynylation procedure (eluent: hexane/ethyl acetate=10/1). The product was obtained as a white solid (22.3 mg, 42% yield). ¹H NMR (600 MHz, Chloroform-d) δ 7.28 (d, J=5.8 Hz, 4H), 7.19 (ddd, J=6.5, 5.7, 2.7 Hz, 1H), 5.92 (d, J=1.3 Hz, 1H), 5.24 (d, J=1.0 Hz, 1H), 4.32 (dd, J=8.6, 6.9 Hz, 1H), 2.57-2.51 (m, 2H), 2.36-2.29 (m, 1H), 2.21 (ddd, J=11.2, 5.2, 2.5 Hz, 1H), 2.00 (s, 3H), 1.97 (s, 3H), 1.70-1.62 (m, 1H), 1.55 (s, 4H), 1.29 (p, J=7.5 Hz, 3H), 1.05 (d, J=7.4 Hz, 18H); ¹³C NMR (151 MHz, CDCl₃) δ 187.67, 187.11, 169.84, 160.59, 158.07, 145.16, 141.70, 141.52, 140.83, 140.44, 128.46, 127.87, 126.50, 116.18, 105.76, 43.38, 31.53, 29.29, 27.05, 26.47, 18.73, 12.59, 12.46, 11.53. HRMS (ESI-TOF) m/z Calcd for C₃₃H₄₅O₄Si⁺ [M+H]⁺ 533.3087, found 533.3094.

### Example 124: (Z)-3-(4-((1-(4-chlorophenyl)-3-methyl-1H-pyrazol-5-yl)methoxy)-3,5-difluorobenzyl)-5-((triisopropylsilyl)methylene)furan-2(5H)-one (71)

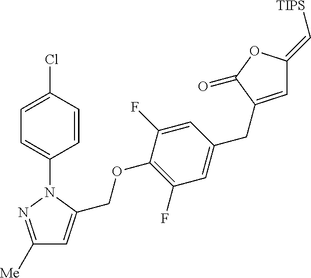

Substrate 61 was alkynylated following the general alkynylation procedure (eluent: hexane/ethyl acetate=10/1). The product was obtained as two inseparable isomers resulted from β-methyl and β-methylene activation. (30.5 mg, 51% combined yield). ¹H NMR (400 MHz, Chloroform-d) δ 7.72-7.64 (m, 2H), 7.50-7.42 (m, 2H), 6.86-6.82 (m, 3H), 6.29 (s, 1H), 5.22 (s, 1H), 5.05 (s, 2H), 3.64 (s, 2H), 2.34 (s, 3H), 1.33-1.28 (m, 3H), 1.09 (d, J=7.7 Hz, 18H); ¹³C NMR (151 MHz, Chloroform-d) δ 170.65, 158.50, 156.26 (d, J=256.7 Hz), 149.60, 138.76, 137.99, 137.86, 133.71 (t, J=9.0 Hz), 133.60, 133.31, 129.45, 125.86, 112.96 (dd, J=18.2, 4.8 Hz), 110.82, 110.31, 65.19, 31.04, 18.84, 13.65, 11.64. HRMS (ESI-TOF) m/z Calcd for C₃₂H₃₈ClF₂N₂O₃Si⁺ [M+H]⁺ 599.2308, found 599.2319.

### Example 125: (Z)-3-((Z)-5-((1R,2S,3S,4S)-3-((hexyloxy)methyl)-7-oxabicyclo[2.2.1]heptan-2-yl)pent-3-en-1-yl)-5-((triisopropylsilyl)methylene)furan-2(5H)-one (7m)

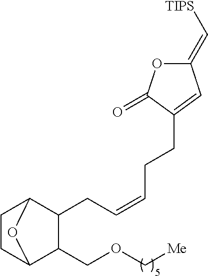

Substrate 6m was alkynylated following the general alkynylation procedure (eluent: hexane/ethyl acetate=10/1). The product was obtained as a white solid (20.7 mg, 39% yield). ¹H NMR (600 MHz, Chloroform-d) δ 6.96 (d, J=1.5 Hz, 1H), 5.46-5.35 (m, 2H), 5.14 (s, 1H), 4.41 (d, J=2.1 Hz, 1H), 4.20-4.16 (m, 1H), 3.44-3.28 (m, 4H), 2.48-2.31 (m, 3H), 2.04 (ddd, J=24.2, 11.4, 7.1 Hz, 3H), 1.83 (ddd, J=11.6, 8.4, 5.8 Hz, 1H), 1.69 (dt, J=7.1, 2.4 Hz, 2H), 1.62-1.26 (m, 14H), 1.07 (d, J=7.5 Hz, 18H), 0.89 (t, J=6.8 Hz, 3H). ¹³C NMR (151 MHz, CDCl3) δ 171.29, 158.86, 137.61, 134.66, 130.71, 128.74, 108.43, 79.99, 79.38, 71.33, 69.89, 46.86, 46.36, 31.70, 29.71, 29.44, 25.91, 25.82, 25.32, 25.03, 22.64, 18.73, 14.07, 12.34, 11.54. HRMS (ESI-TOF) m/z Calcd for C₃₂H₅₅O₄Si⁺ [M+H]⁺ 531.2870, found 531.3874.

### Further Derivatizations of Butenolide Products

The following examples illustrate derivatizations butenolide compounds described above to give additional compounds useful in synthesis.

### Example 126: 3-methylene-4,5,6,7-tetrahydroisobenzofuran-1(3H)-one (8)

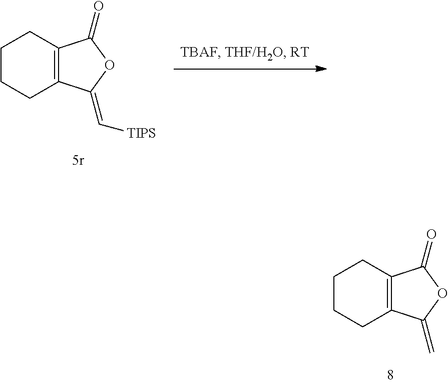

To a solution of 5r (0.1 mmol) in THE (1.0 mL) was added TBAF (3.0 eq), H₂O (10.0 eq), then the mixture was stirred at room temperature until TLC showed that the 3r was fully consumed. The reaction mixture was followed by filtration through a pad of celite, and the solvent was removed under vacuum. The residue was purified by column chromatography to afford 8 (9.9 mg, 66%). ¹H NMR (600 MHz, Chloroform-d) δ 5.03 (d, J=2.6 Hz, 1H), 4.72 (d, J=2.6 Hz, 1H), 2.44-2.39 (m, 2H), 2.34-2.29 (m, 2H), 1.81-1.74 (m, 4H); ¹³C NMR (151 MHz, CDCl₃) δ 169.63, 154.91, 150.94, 129.30, 91.90, 21.45, 21.35, 21.04, 20.07. HRMS (ESI-TOF) m/z Calcd for C₉H₁₁O₂⁺ [M+H]⁺ 151.0759, found 151.0752.

### Example 127: (Z)-3-(iodomethylene)-4,5,6,7-tetrahydroisobenzofuran-1(3H)-one (9)

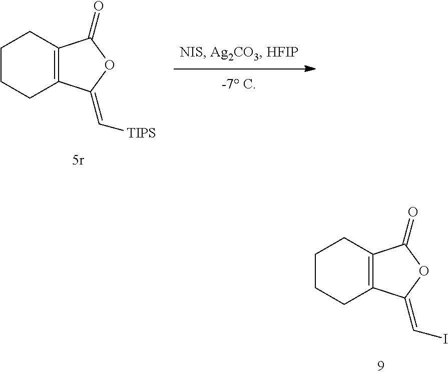

To a solution of 5r (0.1 mmol) in HFIP (0.5 mL) was added NIS (1.3 eq), Ag₂CO₃ (1.0 eq), then the mixture was stirred at −7° C. until TLC showed that the 5r was fully consumed. The reaction mixture was followed by filtration through a pad of celite, and the solvent was removed under vacuum. The residue was purified by column chromatography to afford 9 (20.0 mg, 73%). The product was obtained following the general procedure (eluent: hexane/ethyl acetate=20/1). The product was obtained as a white solid (73% yield). ¹H NMR (500 MHz, Chloroform-d) δ 5.95 (s, 1H), 2.41 (td, J=6.0, 3.0 Hz, 2H), 2.29-2.23 (m, 2H), 1.81-1.72 (m, 4H); ¹³C NMR (126 MHz, CDCl₃) δ 168.40, 156.46, 149.54, 129.71, 56.89, 21.29, 21.27, 21.12, 20.25. HRMS (ESI-TOF) m/z Calcd for C₉H₁₀IO₂⁺ [M+H]⁺ 321.2362, found 321.2367.

### Example 128: 2-(2-(triisopropylsilyl)acetyl)cyclohex-1-ene-1-carboxylic acid (10)

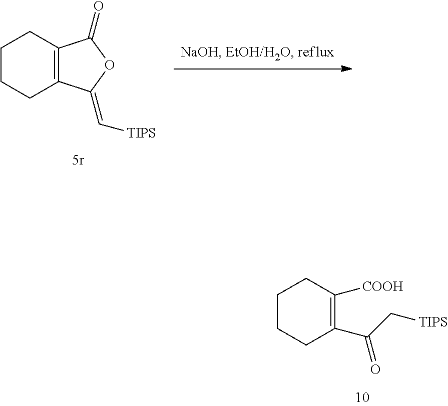

To a solution of 5r (0.1 mmol) in EtOH/H₂O (0.5/0.5 mL) was added NaOH (4.0 eq), then the mixture was heated to reflux until TLC showed that the 5r was fully consumed. The reaction mixture was cooled down to room temperature, and 1M HCl was added until the pH was about 3, followed by filtration through a pad of celite, and the solvent was removed under vacuum. The residue was purified by column chromatography to afford 10 (21.4 mg, 66%). ¹H NMR (600 MHz, Chloroform-d) δ 2.39 (d, J=12.8 Hz, 1H), 2.33 (dd, J=6.2, 3.1 Hz, 2H), 2.23-2.19 (m, 1H), 2.17-2.14 (m, 1H), 2.00 (ddt, J=13.5, 6.3, 3.7 Hz, 1H), 1.86 (ddq, J=13.9, 7.2, 3.3 Hz, 1H), 1.64-1.50 (m, 3H), 1.21-1.16 (m, 3H), 1.08 (d, J=7.3 Hz, 18H); ¹³C NMR (151 MHz, CDCl₃) δ 210.54, 170.39, 144.28, 128.54, 48.31, 26.90, 25.70, 18.78, 18.76, 18.67, 11.50. HRMS (ESI-TOF) m/z Calcd for C₁₈H₃₃O₃Si⁺ [M+H]⁺ 325.2199, found 325.2198.

### Example 129: 4-((triisopropylsilyl)methyl)-5,6,7,8-tetrahydrophthalazin-1-ol (11)

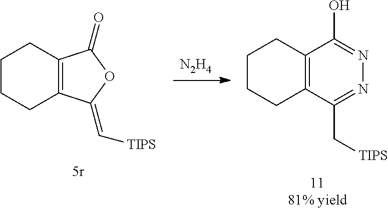

To a solution of 5r (0.1 mmol) in MeOH (1.0 mL) was added aqueous N₂H₄ (2.0 equiv.), the mixture was stirred for 8 hours under room temperature and the solvent was removed under vacuum. The residue was purified by column chromatography to afford 11 (25.9 mg, 81%). The product was obtained following the general procedure (eluent: hexane/ethyl acetate=15/1 to 3/1). The product was obtained as a white solid (81% yield). ¹H NMR (600 MHz, Methanol-d₄) δ 4.88 (s, 2H), 2.61 (tt, J=6.2, 2.0 Hz, 2H), 2.54 (tt, J=6.2, 2.0 Hz, 2H), 1.86-1.74 (m, 4H), 1.24-1.15 (m, 3H), 1.07 (d, J=7.4 Hz, 18H); ¹³C NMR (151 MHz, MeOD) δ 161.86, 149.29, 141.72, 136.30, 26.53, 22.69, 21.28, 20.52, 17.86, 17.85, 12.85, 11.41. HRMS (ESI-TOF) m/z Calcd for C₁₈H₃₃N₂OSi⁺ [M+H]⁺ 321.2362, found 321.2367.

Enumerated references cited in the present disclosure are as follows:
1. K. M. Engle, T.-S. Mei, M. Wasa, J.-Q. Yu, Weak Coordination as Powerful Means for Developing Broadly Useful C—H Functionalization Reactions. Acc. Chem. Res. 45, 788-802 (2012).
2. O. Daugulis, J. Roane, L. D. Tran, Bidentate, monoanionic auxiliary-directed functionalization of carbon-hydrogen bonds. Acc. Chem. Res. 48, 1053-1064 (2015).
3. P. Shen, L. Hu, Q. Shao, K. Hong, J.-Q. Yu, Pd(II)—Catalyzed Enantioselective C(sp³)-H Arylation of Free Carboxylic Acids. J. Am. Chem. Soc. 140, 6545-6549 (2018).
4. S. Gnaim, J. C. Vantourout, F. Serpier, P.-G. Echeverria, P. S. Baran, Carbonyl Desaturation: Where Does Catalysis Stand? ACS Catal. 11, 883-892 (2021).
5. K. B. Sharpless, R. F. Lauer, A. Y. Teranishi, Electrophilic and nucleophilic organoselenium reagents. New routes to α,β-unsaturated carbonyl compounds. J. Am. Chem. Soc. 95, 6137-6139 (1973).
6. H. J. Reich, I. L. Reich, J. M. Renga, Organoselenium chemistry, alpha phenylseleno carbonyl compounds as precursors for alpha, beta-unsaturated ketones and esters. J. Am. Chem. Soc. 95, 5813-5815 (1973).
7. B. M. Trost, T. N. Salzmann, K. Hiroi, New synthetic reactions. Sulfenylations and dehydrosulfenylations of esters and ketones. J. Am. Chem. Soc. 98, 4887-4902 (1976).
8. T. Mukaiyama, J.-I. Matsuo, H. Kitagawa, A New and One-Pot Synthesis of α,β-unsaturated ketones by dehydrogenation of various ketones with N-tert-Butyl phenylsulfin-imidoyl chloride. Chem. Lett. 29, 1250-1251 (2000).
9. K. C. Nicolaou, Y. L. Zhong, P. S. Baran, A new method for the one-step synthesis of α,β-unsaturated carbonyl systems from saturated alcohols and carbonyl compounds. J. Am. Chem. Soc. 122, 7596-7597 (2000).
10. M. Hayashi, M. Shibuya, Y. Iwabuchi, Oxidative conversion of silyl enol ethers to α, β-unsaturated ketones employing oxoammonium salts. Org. Lett. 14, 154-157 (2012).
11. S. Gnaim. Y. Takahira, H. R. Wilke, Z. Yao, J. Li, D. Delbrayelle, P.-G. Echeverria, J. C. Vantourout, P. S. Baran, Electrochemically driven desaturation of carbonyl compounds. Nat. Chem. 13, 367-372 (2021).
12. R. J. Theissen, Preparation of α,β-unsaturated carbonyl compounds. J. Org. Chem. 36, 752-757 (1971).
13. Y. Ito, T. Hirao, T. Saegusa, Synthesis of α,β-unsaturated carbonyl compounds by palladium(II)-catalyzed dehydrosilylation of silyl enol ethers. J. Org. Chem. 43, 1011-1013 (1978).
14. J.-Q. Yu, H.-C. Wu, E. J. Corey, Pd(OH)₂/C Mediated Selective Oxidation of Silyl Enol Ethers by tert-Butylhydroperoxide, a Useful Method for the Conversion of Ketones to α,β-Enones or β-Silyloxy-α,β-enones. Org. Lett. 7, 1415-1417 (2005).
15. M. Chen, G. Dong, Direct catalytic desaturation of lactams enabled by soft enolization. J. Am. Chem. Soc. 139, 7757-7760 (2017).
16. Y. Zhao, Y. Chen, T. R. Newhouse, Allyl-palladium-catalyzed α,β-dehydrogenation of carboxylic acids via enediolates. Angew. Chem. 129, 13302-13305 (2017).
17. A. S. Goldman, A. H. Roy, Z. Huang, R. Ahuja, W. Schinski, M. Brookhart, Catalytic Alkane Metathesis by Tandem Alkane Dehydrogenation-Olefin Metathesis. Science 312, 257-261 (2006).
18. J. G. West, D. Huang, E. J. Sorensen, Acceptorless dehydrogenation of small molecules through cooperative base metal catalysis. Nat Commun, 6, 10093 (2015)
19. R. Giri, N. Maugel, B. M. Foxman, J.-Q. Yu, Dehydrogenation of Inert Alkyl Groups via Remote C—H Activation: Converting a Propyl Group into a π-Allylic Complex. Organometallics. 27, 1667-1670 (2008).
20. P. Wang, M. E. Farmer, X. Huo, P. Jain, P.-X. Shen, M. Ishoey, J. E. Bradner, S. R. Wisniewski, M. D. Eastgate, J.-Q. Yu, Ligand-Promoted Meta-C—H Arylation of Anilines, Phenols, and Heterocycles. J. Am. Chem. Soc. 138, 9269-9276 (2016).
21. P. Wang, P. Verma, G. Xia, J. Shi, J. X. Qiao, S. Tao, P. T. W. Cheng, M. A. Poss, M. E. Farmer, K.-S. Yeung, J.-Q. Yu, Ligand-accelerated non-directed C—H functionalization of arenes. Nature. 551, 489-493 (2017).
22. Chen, Y.-Q. Z. Wang, Y. Wu, S. R. Wisniewski, J. X. Qiao, W. R. Ewing, M. D. Eastgate, J.-Q. Yu Overcoming the Limitations of 7, and S—C—H Arylation of Amines through Ligand Development. J. Am. Chem. Soc. 140, 17884-17894 (2018).
23. Chen, G. W. Gong, Z. Zhuang, M. S. Andra, Y.-Q. Chen, X. Hong, Y.-F. Yang, T. Liu, K. N. Houk, J.-Q. Yu, Ligand-Accelerated Enantioselective Methylene C(sp³)-H Bond Activation. Science. 353, 1023-1027 (2016).
24. Z. Li, Z. Wang, N. Chekshin, S. Qian, J. X. Qiao, P. T. Cheng, K.-S. Yeung, W. R. Ewing, J.-Q. Yu, A tautomeric ligand enables directed C—H hydroxylation with molecular oxygen. Science. In press (2021). (10.1126/science.abg2362)
25. F. Ghiringhelli, A. Uttry, K. K. Ghosh, M. van Gemmeren, Direct β- and γ-C(sp³)-H Alkynylation of Free Carboxylic Acids. Angew. Chem., Int. Ed. 59, 23127-23131 (2020).
26. A. Bauer, N. Maulide, Chemoselective formal β-functionalization of substituted aliphatic amides enabled by a facile stereoselective oxidation event. Chem. Sci., 10, 9836-9840 (2019).
27. S. J. Gharpure, L. N. Nanda, M. K. Shukla, Donor-Acceptor Substituted Cyclopropane to Butanolide and Butenolide Natural Products: Enantiospecific First Total Synthesis of (+)-Hydroxyancepsenolide. Org. Lett. 16, 6424-6427 (2014).
28. P. Tuchinda, J. Udchachon, V. Reutrakul, T. Santisuk, W. C. Taylor, N. R. Farnsworth, J. M. Pezzuto, A. D. Kinghorn, Bioactive Butenolides from Melodorum Fruticosum. Phytochemistry. 30, 2685-2689 (1991).
29. U. A. Pereira, T. A. Moreira, L. C. A. Barbosa, C. R. A. Maltha, I. S. Bomfim, S. S. Maranhão, M. O. Moraes, C. Pessoacd, F. W. A. Barros-Nepomucenoe, Rubrolide Analogues and Their Derived Lactams as Potential Anticancer Agents. Medchemcomm. 7, 345-352 (2016).
30. Y. Liu, F. Chen, Y.-M. He, C. Li, Q.-H. Fan, Enantioselective synthesis of tunable chiral pyridine-aminophosphine ligands and their applications in asymmetric hydrogenation. Org. Biomol. Chem., 17, 5099-5105. (2019).
31. F. Outurquin, C. Paulmier, Straightforward Synthesis of 2-Alkenoic Acids from the Corresponding Saturated Aldehydes. Synthesis 1989, 690-691 (1989).
32. M. V. Popescu, A. Mekereeya, J. V. Alegre-Requena, R. S. Oaton, M. D. Smith, Visible-Light-Mediated Heterocycle Functionalization via Geometrically Interrupted [2+2] Cycloaddition. Angew. Chem. Int. Ed. 59, 23020-23024 (2020).
33. D. M. Roberts, C. Bartel, A. Scott, D. Ivison, T. J. Simpson, R. J. Cox, Substrate selectivity of an isolated enoyl reductase catalytic domain from an iterative highly reducing fungal polyketide synthase reveals key components of programming. Chem. Sci., 8, 1116-1126 (2017).

All patent documents and publications cited in this disclosure are incorporated herein by reference as if fully set forth.

We claim:

1. A process for making a compound of formula (2): 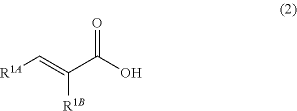
comprising contacting a compound of formula (1): 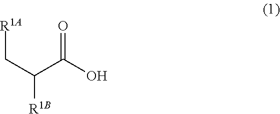
with a source of Pd(II), optionally in the presence of an oxidant, and a ligand of formula (L-1): 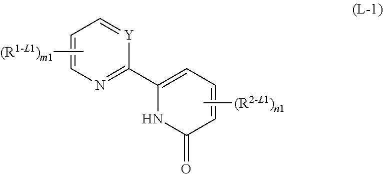
whereby the compound of formula (2) is formed, wherein:
Y is N or CH;
R^{1A} and R^{1B} are independently selected from the group consisting of H, C₁-C₆-alkyl, C₃-C₂₀-cycloalkyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), 5- to 10-membered heteroaryl and —(C₁-C₆-alkyl)-(5- to 10-membered heteroaryl) (wherein 1-4 heteroaryl members are independently selected from N, O, and S);
or R^{1A} and R^{1B}, together with the carbon atoms to which they are bound, form a 5- to 6-membered carbocyclic ring;
wherein R^{1A} and R^{1B}, or the carbocyclic ring that they form, are independently and optionally substituted with one to five substituents selected from the group consisting of halo, C₁-C₆-alkyl, C₁-C₆-haloalkyl, C₁-C₆-alkoxy, —S—(C₁-C₆-alkyl), C₆-C₁₀-aryloxy, —O—(C₁-C₆-alkyl)(C₆-C₁₀-aryl), —S(O)₀₋₂(C₆-C₁₀-aryl) (optionally substituted with C₁-C₆-alkyl), C(O)NR^{A}R^{B}, NR^{A}C(O)O(C₁-C₆-alkyl), —C(O)(C₁-C₆-alkyl), —C(O)O(C₁-C₆-alkyl), —C(O)(C₆-C₁₀-aryl), and —C(O)O(C₆-C₁₀-aryl);
m1 is selected from 0, 1, 2, 3, and 4;
n1 is selected from 0, 1, 2, and 3;
R^{1-L1} and R^{2-L1} are independently selected from the group consisting of —CN, halo, NR^{A}R^{B}, C₁-C₆-alkyl, C₁-C₆-haloalkyl, C₂-C₆-alkenyl, C₂-C₆-alkynyl, C₁-C₆-alkoxy, C₁-C₆-haloalkoxy, C(O)C₁-C₆-alkyl, C(O)NR^{A}R^{B}, S(O)NR^{A}R^{B}, S(O)₂NR^{A}R^{B}, C₃-C₁₄-cycloalkyl, C₆-C₁₀-aryl, C₆-C₁₀-aryloxy, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), wherein
each alkyl, aryl, cycloalkyl, heterocycloalkyl, and heteroaryl moiety is optionally substituted with one to four substituents selected from the group consisting of halo, oxo, C₁-C₆-alkyl, C₁-C₆-haloalkyl, C₁-C₆-alkoxy, C(O)NR^{A}R^{B}, C₁-C₆-alkoxy, C₆-C₁₀-aryl (optionally substituted by one to three halo and C₁-C₆-alkyl), and 5- to 10-membered heteroaryl (wherein 1-4 heteroaryl members are independently selected from N, O, and S; optionally substituted by one to three substituents selected from C₁-C₆-alkyl and 5- to 10-membered heteroaryl);
R^{A} and R^{B} are independently selected from the group consisting of H, C₁-C₆-alkyl, C₁-C₆-haloalkyl, —C₁-C₆-alkyl-C₆-C₁₀-aryl, C(O)C₁-C₆-alkyl, C(O)C₁-C₆-alkyl-C₆-C₁₀-aryl, C(O)OC₁-C₆-alkyl, C₆-C₁₀-aryl (optionally fused to C₃-C₁₄-cycloalkyl that is optionally substituted by one to four halo and C₁-C₆-alkyl), wherein
each aryl is optionally substituted with one to three substituents selected from C₁-C₆-alkyl, halo, C₁-C₆-haloalkyl, and 3- to 14-membered heterocycloalkyl (wherein 1-4 ring members are independently selected from N, O, and S);
each alkyl is optionally substituted with one to three substituents selected from halo, NRR′ (wherein R and R′ are independently selected from H, C₁-C₆-alkyl, C(O)C₁-C₆-alkyl, and C(O)C₆-C₁₀-aryl);
or, optionally, when m1 is 2, then two adjacent R^{1-L1} together with the ring carbon atoms to which they are bound form a fused phenyl ring that is optionally substituted with one to three substituents selected the group consisting of halo, C₁-C₆-alkyl, C₁-C₆-haloalkyl, C₁-C₆-alkoxy, and C₁-C₆-haloalkoxy.

2. The process according to claim 1, wherein R^{1B} is H and R^{1A} is other than H.

3. The process according to claim 1, wherein the compound of formula (1) is one selected from the following table:

| | |
|---|---|
| 1a | 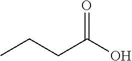 |
| 1b | 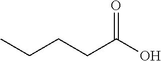 |
| 1c | 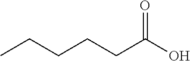 |
| 1d | 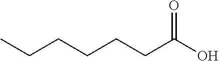 |
| 1e | 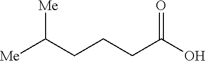 |
| 1f | 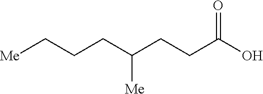 [SMILES: CCCCC(C)CCC(=O)O] |
| 1g | 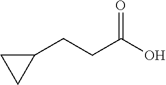 |
| 1h | 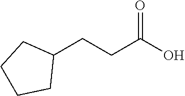 |
| 1i | 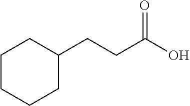 [SMILES: O=C(O)CCC1CCCCC1] |
| 1j | 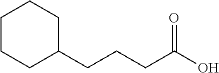 [SMILES: O=C(O)CCCC1CCCCC1] |
| 1k | 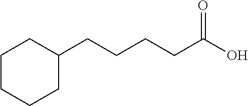 [SMILES: O=C(O)CCCCC1CCCCC1] |
| 1l | 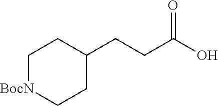 [SMILES: CC(C)(C)OC(=O)N1CCC(CCC(=O)O)CC1] |
| 1m | 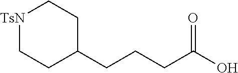 [SMILES: Cc1ccc(S(=O)(=O)N2CCC(CCCC(=O)O)CC2)cc1] |
| 1n | 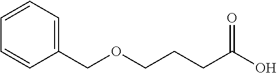 [SMILES: O=C(O)CCCOCc1ccccc1] |
| 1o | 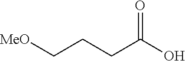 |
| 1p | 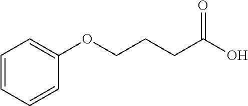 [SMILES: O=C(O)CCCOc1ccccc1] |
| 1q | 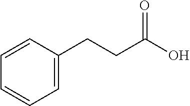 [SMILES: O=C(O)CCc1ccccc1] |
| 1r | 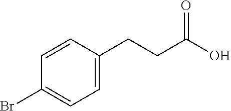 [SMILES: O=C(O)CCc1ccc(Br)cc1] |
| 1s | 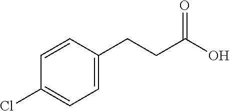 [SMILES: O=C(O)CCc1ccc(Cl)cc1] |
| 1t | 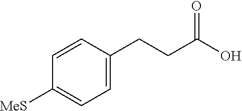 [SMILES: CSc1ccc(CCC(=O)O)cc1] |
| 1u | 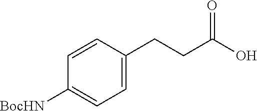 [SMILES: CC(C)(C)OC(=O)Nc1ccc(CCC(=O)O)cc1] |
| 1v | 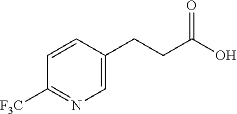 [SMILES: O=C(O)CCc1ccc(C(F)(F)F)nc1] |
| 1w | 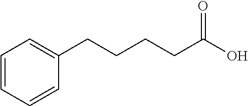 [SMILES: O=C(O)CCCCc1ccccc1] |
| 1x | 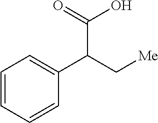 [SMILES: CCC(C(=O)O)c1ccccc1] |
| 1y | 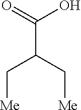 |
| 1z | 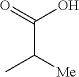 |
| 1aa | 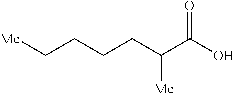 |
| 1ab | 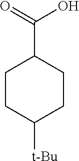 [SMILES: CC(C)(C)C1CCC(C(=O)O)CC1] |
| 1ac | 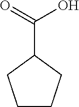 |
| 1ad | 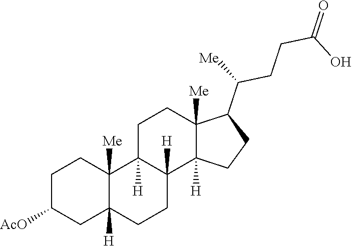 |
| 1ae | 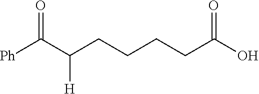 [SMILES: O=C(O)CCCCCC(=O)c1ccccc1] |
| 1af | 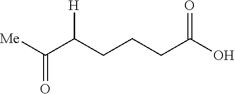 |
| 1ag | 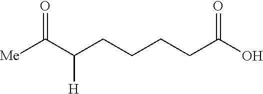 [SMILES: CC(=O)CCCCCC(=O)O] |
| 1ah | 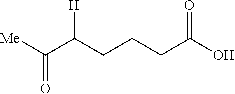 |
| 1ai | 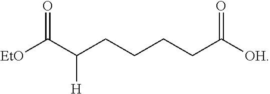 [SMILES: CCOC(=O)CCCCCC(=O)O] |

4. The process according any one of claims 1 to 3, wherein Y is CH.

5. The process according to any one of claims 1 to 4, wherein n1 is 0.

6. The process according to any one of claims 1 to 5, wherein m1 is 0, 1, or 2.

7. The process according to any one of claims 1 to 6, wherein R^{1-L1} is selected from the group consisting of halo, C₁-C₆-alkyl, C₁-C₆-haloalkyl, and C₁-C₆-alkoxy.

8. The process according to any one of claims 1 to 6, wherein m1 is 2, and two adjacent R^{1-L1} together with the ring carbon atoms to which they are bound form an optionally substituted fused phenyl ring.

9. The process according any one of claims 1 to 8, wherein L-1 is one selected from the following table:

| | |
|---|---|
| L8 | 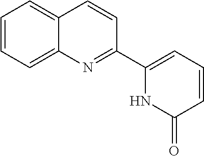 [SMILES: O=c1cccc(-c2ccc3ccccc3n2)[nH]1] |
| L9 | 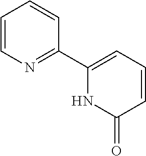 [SMILES: O=c1cccc(-c2ccccn2)[nH]1] |
| L10 | 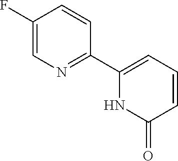 [SMILES: O=c1cccc(-c2ccc(F)cn2)[nH]1] |
| L11 | 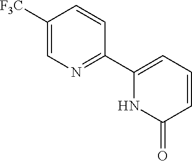 [SMILES: O=c1cccc(-c2ccc(C(F)(F)F)cn2)[nH]1] |
| L12 | 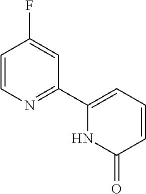 [SMILES: O=c1cccc(-c2cc(F)ccn2)[nH]1] |
| L13 | 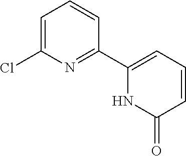 [SMILES: O=c1cccc(-c2cccc(Cl)n2)[nH]1] |
| L14 | 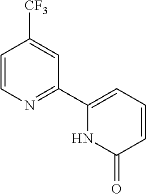 [SMILES: O=c1cccc(-c2cc(C(F)(F)F)ccn2)[nH]1] |
| L15 | 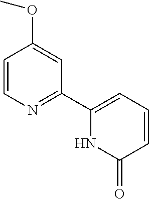 |
| L16 | 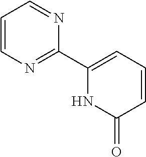 [SMILES: O=c1cccc(-c2ncccn2)[nH]1] |
| L17 | 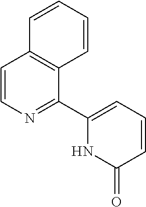 [SMILES: O=c1cccc(-c2nccc3ccccc23)[nH]1] |
| L18 | 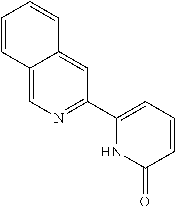 [SMILES: O=c1cccc(-c2cc3ccccc3cn2)[nH]1] |
| L19 | 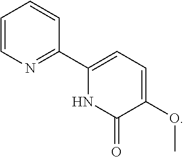 |

10. A process for making a compound of formula (5) or (7): 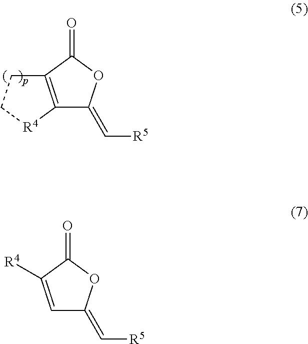
comprising contacting a compound of formula (3) or (6), respectively: 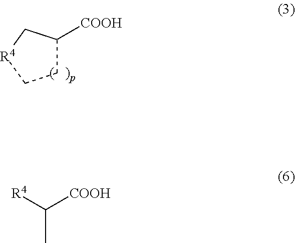
with a source of Pd(II), a ligand of formula (L-2), optionally in the presence of an oxidant: 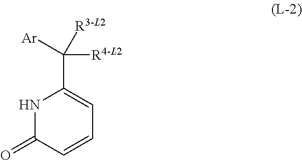
and a compound of formula (A): 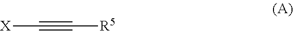
whereby the compound of formula (5) or (7) is formed, wherein
the dotted lines [symbol] represent optional single bonds, and when they are present,
then p is 1, 2, 3, or 4; and
R⁴ is —CH₂—, wherein the resulting 5- to 8-membered ring is cycloalkyl or heterocycloalkyl (wherein 1-2 ring members are independently selected from N, O, and S),
wherein the ring is optionally substituted with one to three substituents selected from halo, C₁-C₆-alkyl, C₁-C₆-haloalkyl, C₁-C₆-alkoxy, —S(O)₀₋₂(C₆-C₁₀-aryl) (optionally substituted with C₁-C₆-alkyl), and C₆-C₁₀-aryl (optionally and independently substituted by one to three halo, C₁-C₆-alkyl, and C₁-C₆-haloalkyl);
when the bonds [symbol] are not present, then R⁴ is selected from the group consisting of C₁-C₂₀-alkyl, C₃-C₁₄-cycloalkyl, —(C₁-C₆-alkyl)-(C₃-C₁₄-cycloalkyl), C₆-C₁₀-aryl, —(C₁-C₆-alkyl)-(C₆-C₁₀-aryl), 3- to 14-membered heterocycloalkyl and —(C₁-C₆-alkyl)-(3- to 14-membered heterocycloalkyl) (wherein 1-4 ring members are independently selected from N, O, and S), 5- to 10-membered heteroaryl and —(C₁-C₆-alkyl)-(5- to 10-membered heteroaryl) (wherein 1-4 heteroaryl members are independently selected from N, O, and S),
wherein R⁴ or the ring in which R⁴ is a member is independently and optionally substituted with one to five substituents selected from the group consisting of halo, C₁-C₆-alkyl, C₁-C₆-haloalkyl, C₁-C₆-alkoxy, —S—(C₁-C₆-alkyl), C₆-C₁₀-aryloxy, —O—(C1-C6-alkyl)(C₆-C₁₀-aryl), —S(O)₀₋₂(C₆-C₁₀-aryl) (optionally substituted with C₁-C₆-alkyl), C(O)NR^{A}R^{B}, NR^{A}C(O)O(C₁-C₆-alkyl), —C(O)(C₁-C₆-alkyl), —C(O)O(C₁-C₆-alkyl), —C(O)(C₆-C₁₀-aryl), and —C(O)O(C₆-C₁₀-aryl);
R⁵ is selected from the group consisting of —SiR₃ and —CH(OSiR₃)R′, wherein
each R and R′ is independently selected from the group consisting of C₁-C₆-alkyl, C₁-C₆-alkoxy, C₁-C₆-haloalkyl, and C₃-C₁₄-cycloalkyl;
Ar is 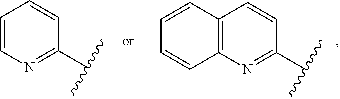 [SMILES: *c1ccccn1 ; *c1ccc2ccccc2n1]
and is optionally substituted with one to three substituents selected from the group consisting of halo, C₁-C₆-alkyl, and C₁-C₆-alkoxy;
R^{3-L2} and R^{4-L2} are independently selected from the group consisting of H, OH, halo, C₁-C₆-alkyl, C₁-C₆-haloalkyl, C₃-C₁₄-cycloalkyl, —(C₁-C₆-alkyl)-(C₃-C₁₄-cycloalkyl), C₆-C₁₀-aryl, and —(C₁-C₆-alkyl)-(C₆-C₁₀-aryl),
or R^{3-L2} and R^{4-L2}, together with the carbon atom to which they are bound, form a 5- or 6-membered cycloalkyl,
or one of R^{3-L2} and R^{4-L2}, together with the 3-pyridyl carbon when Ar is pyridyl, form a 5- to 6-membered cycloalkyl fused to the pyridyl;
wherein R^{3-L2} and R^{4-L2}, or a ring in which either is a member, are independently and optionally substituted by one to three substituents selected from the group consisting of halo, C₁-C₆-alkyl, C₁-C₆-haloalkyl, and C₁-C₆-alkoxy.

11. The process according to claim 10, wherein the contacting is between a compound of formula (3), source of Pd(II), ligand of formula (L-2), and compound of formula (A).

12. The process according to claim 10 or 11, wherein the optional bonds [symbol] are absent.

13. The process according to claim 10 or 11, wherein the optional bonds [symbol] are present.

14. The process according to any one of claims 10, 11, and 13, wherein p is 1 or 2.

15. The process according to any one of claims 10 to 14, wherein the compound of formula (3) is one selected from the following table:

| | |
|---|---|
| 3a | 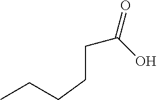 |
| 3b | 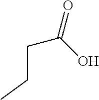 |
| 3c | 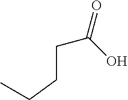 |
| 3d | 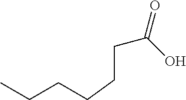 |
| 3e | 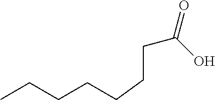 |
| 3f | 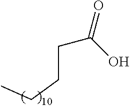 |
| 3g | 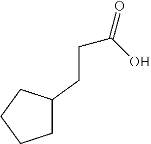 |
| 3h | 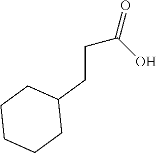 [SMILES: O=C(O)CCC1CCCCC1] |
| 3i | 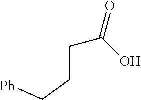 [SMILES: O=C(O)CCCc1ccccc1] |
| 3j | 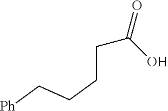 [SMILES: O=C(O)CCCCc1ccccc1] |
| 3k | 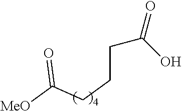 |
| 3l | 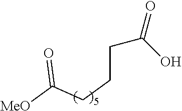 |
| 3m | 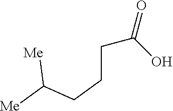 |
| 3n | 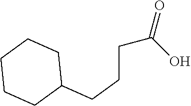 [SMILES: O=C(O)CCCC1CCCCC1] |
| 3o | 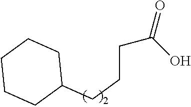 |
| 3p | 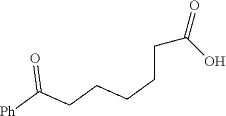 [SMILES: O=C(O)CCCCCC(=O)c1ccccc1] |
| 3q | 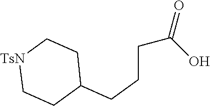 [SMILES: Cc1ccc(S(=O)(=O)N2CCC(CCCC(=O)O)CC2)cc1] |
| 3r | 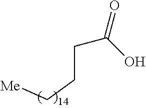 |
| 3s | 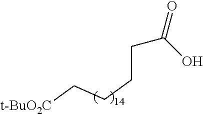 |
| 3t | 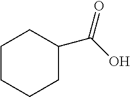 |
| 3u | 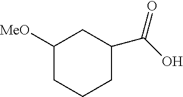 [SMILES: COC1CCCC(C(=O)O)C1] |
| 3v | 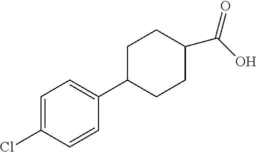 [SMILES: O=C(O)C1CCC(c2ccc(Cl)cc2)CC1] |
| 3w | 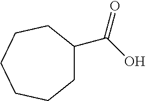 |
| 3x | 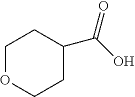 |
| 3y | 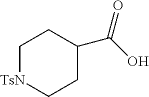 [SMILES: Cc1ccc(S(=O)(=O)N2CCC(C(=O)O)CC2)cc1] |
| 3z | 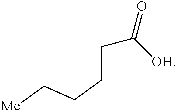 |

16. The process according to claim 10, wherein the contacting is between a compound of formula (6), source of Pd(II), ligand of formula (L-2), and compound of formula (A).

17. The process according to any one of claims 10 to 16, wherein the compound of formula (6) is one selected from the following table:

| | |
|---|---|
| 6a | 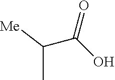 |
| 6b | 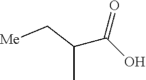 |
| 6c | 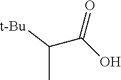 |
| 6d | 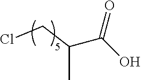 |
| 6e | 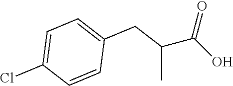 |
| 6f | 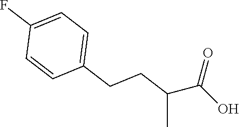 |
| 6g | 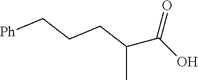 |
| 6h | 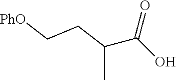 |
| 6i | 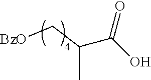 |
| 6j | 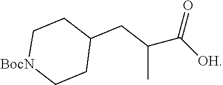 [SMILES: CC(CC1CCN(C(=O)OC(C)(C)C)CC1)C(=O)O] |

18. The process according to any one of claims 10 to 17, wherein Ar is optionally substituted

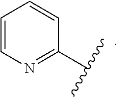 [SMILES: *c1ccccn1]

19. The process according to any one of claims 10 to 17, wherein Ar is optionally substituted

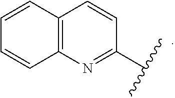 [SMILES: *c1ccc2ccccc2n1]

20. The process according to any one of claims 10 to 19, wherein R^{3-L2} and R^{4-L2} are independently selected from optionally substituted C₁-C₆-alkyl and —(C₁-C₆-alkyl)-(C₆-C₁₀-aryl).

21. The process according to any one of claims 10 to 20, wherein the ligand of formula (L-2) is one selected from the following table:

| | |
|---|---|
| L20 | 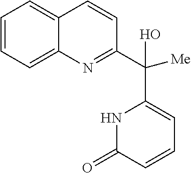 [SMILES: CC(O)(c1ccc2ccccc2n1)c1cccc(=O)[nH]1] |
| L5 | 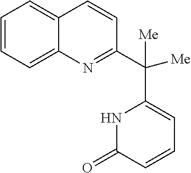 [SMILES: CC(C)(c1ccc2ccccc2n1)c1cccc(=O)[nH]1] |
| L21 | 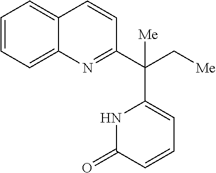 [SMILES: CCC(C)(c1ccc2ccccc2n1)c1cccc(=O)[nH]1] |
| L22 | 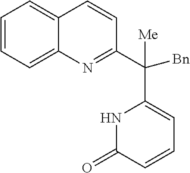 [SMILES: CC(Cc1ccccc1)(c1ccc2ccccc2n1)c1cccc(=O)[nH]1] |
| L23 | 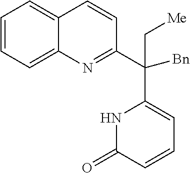 [SMILES: CCC(Cc1ccccc1)(c1ccc2ccccc2n1)c1cccc(=O)[nH]1] |
| L24 | 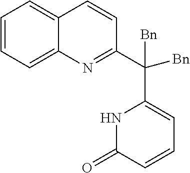 [SMILES: O=c1cccc(C(Cc2ccccc2)(Cc2ccccc2)c2ccc3ccccc3n2)[nH]1] |
| L25 | 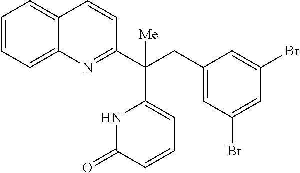 [SMILES: CC(Cc1cc(Br)cc(Br)c1)(c1ccc2ccccc2n1)c1cccc(=O)[nH]1] |
| L26 | 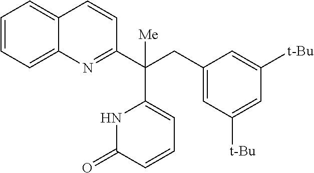 [SMILES: CC(C)(C)c1cc(CC(C)(c2ccc3ccccc3n2)c2cccc(=O)[nH]2)cc(C(C)(C)C)c1] |
| L27 | 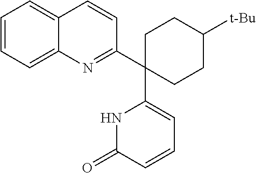 [SMILES: CC(C)(C)C1CCC(c2ccc3ccccc3n2)(c2cccc(=O)[nH]2)CC1] |
| L28 | 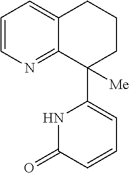 [SMILES: CC1(c2cccc(=O)[nH]2)CCCc2cccnc21] |
| L29 | 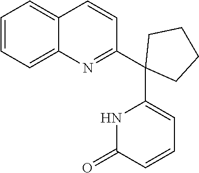 [SMILES: O=c1cccc(C2(c3ccc4ccccc4n3)CCCC2)[nH]1] |
| L30 | 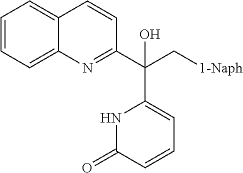 |
| L31 | 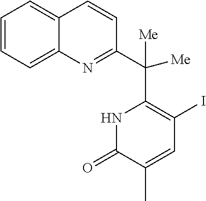 [SMILES: Cc1cc(I)c(C(C)(C)c2ccc3ccccc3n2)[nH]c1=O] |
| L32 | 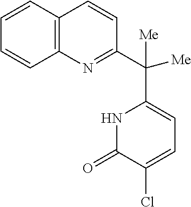 [SMILES: CC(C)(c1ccc2ccccc2n1)c1ccc(Cl)c(=O)[nH]1] |
| L33 | 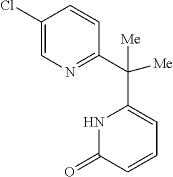 [SMILES: CC(C)(c1ccc(Cl)cn1)c1cccc(=O)[nH]1] |
| L34 | 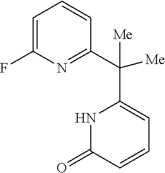 [SMILES: CC(C)(c1cccc(F)n1)c1cccc(=O)[nH]1] |
| L35 | 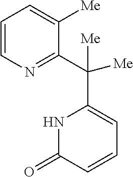 [SMILES: Cc1cccnc1C(C)(C)c1cccc(=O)[nH]1] |
| L36 | 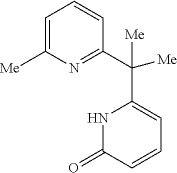 [SMILES: Cc1cccc(C(C)(C)c2cccc(=O)[nH]2)n1] |
| L37 | 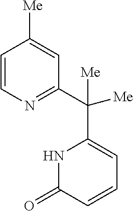 [SMILES: Cc1ccnc(C(C)(C)c2cccc(=O)[nH]2)c1] |
| L38 | 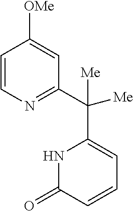 [SMILES: COc1ccnc(C(C)(C)c2cccc(=O)[nH]2)c1] |
| L39 | 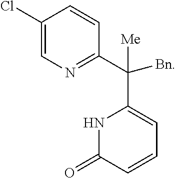 [SMILES: CC(Cc1ccccc1)(c1ccc(Cl)cn1)c1cccc(=O)[nH]1] |

22. The process according to any one of claims 10 to 21, wherein R⁵ is —SiR₃.

23. The process according to any one of claims 10 to 22, wherein R is C₁-C₆-alkyl.

24. The process according to any one of claims 10 to 23, wherein X is Br.

25. The process according to any one of claims 1 to 24, wherein the source of Pd(II) is a Pd(II) salt.

26. The process according to any one of claims 1 to 25, wherein the oxidant is present and is a silver salt, an organic peroxide, organic hydroperoxide, O₂, or combination thereof.

27. The process according to any one of claims 1 to 26, wherein the oxidant is a silver salt.

28. The process according to claim 27, wherein the silver salt is at least one selected from the group consisting of Ag₂CO₃, AgOAc, silver pivalate (AgOPiv), and Ag₂O.

29. The process according to claim 27 or 28, wherein the silver salt is Ag₂CO₃.

30. The process according to any one of claims 1 to 26, wherein the oxidant is O₂.

31. The process according to any one of claims 1 to 26, wherein the oxidant is an organic peroxide or organic hydroperoxide of the formula R′−O—O—R″, wherein R′ and R″ are chosen from H, C₁-C₆-alkyl, —(C₁-C₆-alkyl)-(C₆-C₁₀-aryl), —C(O)(C₁-C₆-alkyl), and —C(O)(C₆-C₁₀-aryl).

32. The process according to claim 31, wherein one of R′ and R″ is H.

33. The process according to any one of claims 1 to 26, wherein the oxidant is selected from the group consisting of tert-butylhydroperoxide (TBHP), cumene hydroperoxide (CMHP), acetyl-tert-butylperoxide (AcOOtBu), benzoyl tert-butylperoxide (BzOOtBu), dibenzoylperoxide (BzOOBz), and di-tertbutyl peroxide (tBuOOtBu).

34. The process according to any one of claims 1 to 26, wherein the oxidant is tert-butylhydroperoxide (TBHP).

35. The process according to any one of claims 1 to 34, wherein the contacting further occurs in the presence of at least one non-nucleophilic base.

36. The process according to claim 34, wherein the non-nucleophilic base is at least one selected from the group consisting of KOAc, NaOAc, NaHCO₃, Na₂CO₃, NaH₂PO₄, Na₂HPO₄, Na₃PO₄, Li₂CO₃, LiOAc, Li₃PO₄, KOAc, KHCO₃, K₂CO₃, K₃PO₄, K₂HPO₄, KH₂PO₄, and CsOAc.
